(19) United States
(12) Patent Application Publication
(10) Pub. No.: US 2004/0161765 A1
(43) Pub. Date: Aug. 19, 2004

(54) Methods and compositions for identifying disease genes using nonsense-mediated decay inhibition

(76) Inventors: Harry C. Dietz, Towson, MD (US); Eric Noensie, New York, NY (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/474,553

(22) PCT Filed: Apr. 12, 2002
(86) PCT No.: PCT/US02/11688

(51) Int. Cl. C12Q 1/68

(52) U.S. Cl. 435/6

(57) ABSTRACT

The invention provides compositions and methods for diagnostic and therapeutic applications of gene identification by nonsense-mediated decay inhibition (GINI). The approach allows the rapid identification of the genes and genetic lesions responsible for monogenic as well as polygenic human genetic disorders. In addition, the approach supports diagnostic typing and therapeutic selection for human cancers and other disorders arising as a result of gene mutations.

### 1. BACKGROUND OF THE INVENTION

A variety of inherited and acquired diseases are associated with genetic variations such as point mutations, deletions and insertions. Some of these variations are directly associated with the presence of disease, while others correlate with disease risk and/or prognosis. It is estimated that there are more than 500 human genetic diseases which result from mutations in single genes (see e.g. Antonarakis (1989) New Engl J Med 320: 153-63). These include Marfan syndrome, cystic fibrosis, muscular dystrophy, alpha. 1-antitrypsin deficiency, phenylketonuria, sickle cell anemia, and various other hemoglobinopathies. Furthermore, inheritance of an increased susceptibility to several common polygenic conditions, such as atherosclerotic heart disease, have also been associated with the inheritance of particular genetic alterations.

While the defective genes underlying several monogenic human genetic disease have been mapped, cloned and characterized, many other human disease genes, particularly those contributing to polygenic conditions, remain to be characterized. The traditional approach for identifying human genetic disease-causing genes involves an immense amount of effort in identifying affected families, mapping the chromosomal segments associated with inheritance of the disease, cloning the affected locus, identifying the precise gene affected and confirming the disease-causing lesion. As a result, advances in our understanding of the molecular etiology of other human genetic diseases are being made only slowly and, indeed, many such diseases with limited occurrence or affecting only economically-disadvantaged human populations may be neglected entirely due to limited resources for research.

Furthermore, a very prevalent human disease, cancer, is thought to arise via human somatic mutations—i.e. the accumulation of genetic lesions in genes involved in cellular proliferation or differentiation. The ras proto-oncogenes, K-ras, N-ras and H-ras, and the p53 tumor suppressor gene are examples of genes which are frequently mutated in human cancers. Specific mutations in these genes leads to an increase in transforming potential. The ability to identify the precise genetic lesion giving rise to transformation in a given patient would be invaluable in the clinic for assessing disease risk, diagnosing disease, predicting a patient's prognosis or response to therapy, and monitoring a patient's progress. Still further, identification of the lesion(s) giving rise to cancerous transformation would allow for the design and/or selection of therapeutics specifically targeted to the appropriate biological transforming activities and pathways. The introduction of appropriate genetic tests for such applications, however, will depend on the development of simple, inexpensive, and rapid assays for detecting genetic variations giving rise to cancer.

Therefore, it would be desirable to have a method for quickly identifying the gene defect(s) associated with a given monogenic or polygenic human genetic disease or disorder. Identification of the affected gene(s) and causative lesion(s) would support the development of therapeutic treatments—e.g. through gene replacement therapy or the rational design of targeted pharmacological agents. Preferably, such a method would also support diagnostic applications, so that affected individuals could be rapidly identified to assist in their therapeutic treatment (i.e. pharmacogenetic applications) or for use in human genetic counseling.

### 2. SUMMARY OF THE INVENTION

In one embodiment, the invention provides diagnostic methods, composition and devices for identifying a cellular gene carrying a mutation that causes nonsense-mediated premature protein termination in a cell or cell population and which results in nonsense-mediated mRNA decay (NMD) of the resulting mutant transcript. In preferred embodiments, the gene mutation (or genetic mutation) is associated with (e.g. causes or contributes to or is linked to a gene which results in) a disease or disorder. The methods of the invention are referred to generally as GINI (for Gene Identification by Nonsense-mediated mRNA decay Inhibition).

In a preferred embodiment, the invention provides a method in which a cell or cell population which is suspected of carrying a disease-associated genetic mutation is analyzed by detecting the level of expression of a gene or genes being expressed (e.g. mRNA levels). The levels thus detected are the control levels. Preferably, the cells are derived from a subject (e.g. a human subject) that may carry a genetic mutation associated with a disease or disorder. Preferably, a plurality of genes are assessed by methods which allow detection of the expression of many different genes at once (e.g. microarray analysis), although methods involving the detection of one or more disease gene-candidates (e.g. Northern blot analysis) are also within the scope of the invention. In the next step, the same cell or cell population (e.g. not necessarily the same cells, but genetically-identical cells, such as cell derived from the same host) are treated to inhibit nonsense-mediated mRNA decay and the level of expression of the gene or genes being expressed (e.g. mRNA levels) are measured again. The levels detected are the NMD-inhibited experimental levels. By detecting an increase in the level of expression of a particular gene in the NMD-inhibited experimental cell(s) in comparison to the control cell(s), the invention allows for the identification of genes that carry a mutation that causes nonsense-mediated premature protein termination, and resulting NMD of the mutant mRNA, in the cell or cell population. Such mutant genes may directly cause a disease or disorder or may contribute to the disease or disorder (e.g. polygenic disease/disorders) or may be merely associated with the disease or disorder (e.g. linked to a disease-causing gene). In preferred embodiments, the disease or disorder has been characterized genetically in a subject population such that some information—e.g. likely chromosomal location of the disease-causing gene(s) or likely molecular characteristics (e.g. gene or encoded protein sequence, motif or pathway-association) is available. Where such additional information is available, it may be used in concert with the identity of the candidate genetic mutation-carrying gene(s) identified as being up-regulated by the inhibition of nonsense-mediated mRNA decay in order to further identify the disease-causing genetic defect.

In preferred embodiments of the invention, NMD is inhibited in a subject cell or cell population or in vitro reconstituted cell system by contacting the cell or cell population or in vitro reconstituted cell system with a pharmacological agent that interferes with the nonsense-mediated decay pathway. In preferred embodiments, the pharmacological agent is an inhibitor of protein translation such as emetine, anisomycin, cycloheximide, pactamycin, puromycin, gentamicin, neomycin, or paromomycin. In other preferred embodiments, nonsense-mediated mRNA decay is inhibited in the test cell or cell population by RNA interference targeting one or more components of the NMD pathway. In this embodiment siRNA (short inhibitory RNAs) comprising a sequence of consecutive nucleotides present in a component of the NMD pathway are introduced into the test cell. In particularly preferred embodiments, the siRNAs include a sequence of consecutive nucleotides present in either or both RENT1 and RENT2-components of the NMD pathway. Preferably the RENT1 siRNAs include SEQ ID Nos. 1 and 2, although other double-stranded RNA sequences of about 20 nucleotides may be obtained from e.g. the RENT1 sequence represented in SEQ ID No. 5. Preferably the RENT2 siRNAs include SEQ ID Nos. 3 and 4, although other double-stranded RNA sequences of about 20 nucleotides may be obtained from e.g. the RENT2 sequence represented in SEQ ID Nos. 7 and 8. In another preferred embodiment, nonsense-mediated mRNA decay may be inhibited in the cell or cell population by introduction of a dominant negative RENT1 or RENT2 polypeptide—e.g. a dominant negative RENT1 which carries an arg to cys mutation at the RENT1 amino acid residue 843 (e.g. the dominant negative RENT1 represented by the polypeptide of SEQ ID No. 6). In other preferred embodiments, nonsense-mediated mRNA decay is inhibited in the cell or cell population by introduction of an antisense nucleic acid directed against a component of the NMD pathway such as a RENT1 mRNA or a RENT2 mRNA. In addition, nonsense-mediated mRNA decay may be inhibited by introduction of a ribozyme directed against a RENT1 mRNA or a RENT2 mRNA or other NMD pathway component.

In certain embodiments, the cellular gene detected is an oncogene or a tumor suppressor gene such as ATM, BRCA1, HER2 or p53. In other embodiments, it is a gene associated with a heritable genetic disorder such as FBN1 (fibrillin) which is associated with Marfan syndrome or OAT (omithine aminotransferase) which is associated with gyrate dystrophy. In other embodiments, genes likely to be associated with a disease or disorder based upon chromosomal location or molecular characteristics are utilized in the invention.

In particularly preferred embodiments of the invention, the level of expression of the candidate gene is detected by a method such as microarray analysis, quantitative pcr, SAGE analysis, Northern blot analysis or dot blot analysis.

The invention also provides computer-readable media, such as a computer-readable medium that contains a plurality of digitally encoded information representing the genes having the strongest background response to inhibition of nonsense-mediated mRNA decay such as early growth response protein 1, hormone receptor (growth factor-inducible nuclear protein N10), putative DNA-binding protein A20, early growth response protein 2, p55-c-fos proto-oncogene, major histocompatibility complex enhancer-binding protein MAD3, gem GTPase, transcription factor RELB, spermidine/spermine N1-acetyltransferase, thyroid hormone receptor, alpha; DNA-damage-inducible transcript 1, dual-specificity protein phosphatase PAC-1, interferon regulatory factor 1, interleukin 1, alpha, V-abl Abelson murine leukemia viral oncogene homolog 2, DEC1, diphtheria toxin receptor, early growth response protein 3, putative transmembrane protein NMA, peptidyl-prolyl cis-trans isomerase, IAP homolog C MIHC, thyroid receptor interactor TRIP9, natural killer cells protein 4 precursor and small inducible cytokine A2. The genes with the strongest background response to inhibition of nonsense-mediated mRNA decay may also be represented by the GenBank Accession Nos.: X52541, D49728, M59465, J04076, M69043, U10550, M83221, U40369, M24898, L24498, L11329, X14454, M28983, M35296, AB004066, M60278, X63741, U23070, M80254, U37546, L40407, M59807 and M26683 respectively. In preferred embodiments, the invention includes a step in which a candidate mutant gene up-regulated by NMD inhibition is discounted or otherwise less preferred if it corresponds to one of the foregoing genes which have the strongest background (nonspecific) response to inhibition of NMD.

In another preferred embodiment, the invention provides a method of identifying a candidate mutant gene in a cell or cell population that carries a genetic mutation that causes nonsense-mediated mRNA decay by first providing a cell or cell population that carries the genetic mutation and measuring the level of expression of one or more genes in the cell(s). The level of expression thus measured is the control level of expression of each gene. Next, the level of expression of the same gene(s) in the same (e.g. genetically identical) cell(s) is measured under conditions in which nonsense-mediated mRNA decay is inhibited. The data from the control and NMD-inhibited measurements is compared and a gene in which which the control level of expression of the gene is lower than the level of expression under NMD-inhibiting conditions is selected. The resulting selected gene is a candidate mutant gene for the genetic mutation that causes nonsense-mediated mRNA decay in the cell(s). In preferred embodiments, the genetic mutation causes or contributes to a human genetic disease or disorder such as cancer or a heritable human genetic disease such as Marfan syndrome. In preferred embodiments, the gene selected is other than early growth response protein 1, hormone receptor (growth factor-inducible nuclear protein N10), putative DNA-binding protein A20, early growth response protein 2, p55-c-fos proto-oncogene, major histocompatibility complex enhancer-binding protein MAD3, gem GTPase, transcription factor RELB, spermidine/spermine N1-acetyltransferase, thyroid hormone receptor, alpha; DNA-damage-inducible transcript 1, dual-specificity protein phosphatase PAC-1, interferon regulatory factor 1, interleukin 1, alpha, V-abl Abelson murine leukemia viral oncogene homolog 2, DEC1, diphtheria toxin receptor, early growth response protein 3, putative transmembrane protein NMA, peptidyl-prolyl cis-trans isomerase, IAP homolog C MIHC, thyroid receptor interactor TRIP9, natural killer cells protein 4 precursor and small inducible cytokine A2 (i.e. corresponding to GenBank Accession Nos.: X52541, D49728, M59465, J04076, M69043, U10550, M83221, U40369, M24898, L24498, L11329, X14454, M28983, M35296, AB004066, M60278, X63741, U23070, M80254, U37546, L40407, M59807 and M26683) which genes show high background (i.e. nonspecific) response in the GINI assay.

In another preferred embodiment, the invention provides for compositions and methods of subtractive hybridization for identifying a candidate mutant gene in a cell line or cell population that carries a genetic mutation that causes nonsense-mediated mRNA decay. In this aspect of the invention, a cell population or a cell line that carries a genetic mutation is used to form a first cDNA population from the cellular mRNA that has been expressed by the cell(s) under conditions in which nonsense-mediated mRNA decay is inhibited, and then a second cDNA population is created from mRNA that has been expressed by the cell(s) under control conditions in which nonsense-mediated mRNA decay is not inhibited. By removing from the first cDNA population at least a portion of the cDNA common to the first and second populations (i.e. subtractive hybridization) an enriched cDNA population coding for genes that are differentially stabilized by inhibition of nonsense-mediated mRNA decay is provided. From this enriched population, a candidate mutant gene carrying a genetic mutation is readily identified (e.g. with additional disease-gene information such as chromosomal location of the defective gene or likely molecular characteristics of the defective gene). In preferred embodiments of this aspect of the invention, the invention thus provides library (e.g. one obtained by subtractive hybridization) that includes multiple cDNA sequences that code for genes that are differentially stabilized by inhibition of nonsense-mediated mRNA decay.

In yet another preferred embodiment, the invention provides a method of determining whether a cellular phenotype that is associated with a disease or disorder that results from a nonsense mutation. In this aspect of the invention, a cell or cell population that has a cellular phenotype that is associated with a disease or disorder is utilized (e.g. for cystic fibrosis the loss of cAMP-activated chloride channel) and the cellular phenotype is observed under control conditions (i.e. in the absence of inhibition of NMD). Next, nonsense mediated mRNA decay is inhibited in the cell(s) and any alteration in the cellular phenotype associated with inhibition of NMD is detected. Detection of an alteration in the cellular phenotype following the inhibition of nonsense mediated decay indicates that the disease or disorder results from a genetic mutation causing nonsense-mediated mRNA of the affected genes. Notably, inhibition of NMD may either exacerbate the cellular disease/disorder phenotype (e.g. by stabilization of mutant messages encoding defective (e.g. dominant negative) truncated proteins) or the cellular disease or disorder phenotype may lessen following inhibition of NMD (e.g. where the stabilized mRNA encodes a fully or partially functional (albeit truncated) polypeptide— as in the case of certain cystic fibrosis-causing mutations).

### 3. BRIEF DESCRIPTION OF THE FIGURES

FIG. 1 shows the effects of various translation-inhibiting drugs on nonsense-carrying and wild-type mRNA transcripts.

FIG. 2 shows that emetine stabilizes nonsense-carrying mRNA transcripts

FIG. 3 shows a comparison of transcript-specific responses to emetine in various cell lines.

FIG. 4 shows the response of FIP2 transcripts to emetine.

FIG. 5 shows the specific inhibition of nonsense-mediated decay using RNA interference (RNAi) to inhibit rent1 or rent2 expression.

### 4. DETAILED DESCRIPTION OF THE INVENTION

4.1. General

In general, the invention provides methods and compositions for the identification of genes underlying a disease or disorder and for the detection of the molecular alteration underlying the disease or disorder phenotype. Currently, the identification of a disease gene requires a tremendous amount of information regarding the position of a disease locus and the functional properties of proteins encoded by candidate genes. These limitations preclude the use of standard methods to identify disease genes that cause relatively rare disorders. The method of the invention provides a powerful mechanism to associate a nucleotide sequence with a cellular or clinical phenotype of interest, even in the absence of any information regarding gene location or the function of the encoded protein.

The method of the invention is generally referred to as GINI (for Gene Identification by Nonsense-mediated decay Inhibition). It is estimated that at least one-third of the mutations underlying monogenic and polygenic human disorders result in premature termination codons, which subsequently lead to the rapid breakdown of the mutant mRNA by a pathway called the nonsense-mediated decay pathway (NMD pathway) (see e.g. Frischmeyer and Dietz (1999) Hum Mol Genet 8: 1893-1900). The invention provides for methods and compositions to identify such disease-causing mutant gene transcripts by inhibiting their nonsense-mediated decay. This inhibition of NMD thereby selectively stabilizes mutant transcripts affected by the nonsense-mediated decay pathway and allows for their rapid identification in a sample derived from a cell expressing the mutant gene. The selectively stabilized mutant transcript is then distinguished and identified by screening methods such as by microarray analysis (e.g. cDNA microarray analysis)—which allows for rapid screening of a large number of potentially affected genes. Alternatively, relatively smaller numbers of potentially affected genes may be screened through the GINI approach using, e.g. traditional Northern or dot blot analysis. To distinguish stabilized nonsense transcripts from background transcripts that are nonspecifically upregulated, expression changes are measured in control and disease cell lines with such cDNA microarrays. Indeed, the invention provides the identity of a multiplicity of genetic loci which contribute to a background (i.e. false positive) response to the inhibition of NMD (see Table 1) thereby facilitating identification of the bona fide disease-causing mutant gene transcript. The responsive, non-background transcripts may be ranked by a nonsense enrichment index (NEI), which relates expression changes for a given transcript in NMD-inhibited control and patient cell lines.

In preferred embodiments, GINI strategy eliminates the confounding effects of inter-individual variation in gene expression and secondary changes in gene expression that are caused by the disease process. This approach allows the true disease gene to be ranked in the top one percent of candidates. Furthermore, in particularly preferred embodiments, the GINI method is combined with adjunct information, including the inferred or known biological function of the disease-causing defect or its chromosomal map position. Accordingly, the GINI method allows for rapid and accurate identification of gene defects that cause or contribute to a variety of human diseases and disorders. The GINI method may also be applied to the identification of disease-causing genes in model organisms.

4.2. Definitions

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

The phrase “a corresponding normal cell of” or “normal cell corresponding to” or “normal counterpart cell of” a diseased cell refers to a normal cell of the same type as that of the diseased cell. For example, a corresponding normal PBMC of a subject having R.A. is a PBMC of a subject not having R.A.

An “address” on an array, e.g., a microarray, refers to a location at which an element, e.g., an oligonucleotide, is attached to the solid surface of the array.

The term “agonist,” as used herein, is meant to refer to an agent that mimics or up-regulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

“Amplification,” as used herein, relates to the production of additional copies of a nucleic acid sequence. Amplification is generally carried out using polymerase chain reaction (PCR) technologies well known in the art. (Dieffenbach, C. W. and G. S. Dveksler (1995) PCR Primer, a Laboratory Manual, Cold Spring Harbor Press, Plainview, N.Y.)

“Antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide or enzyme substrate. An antagonist can also be a compound that downregulates expression of a gene or which reduces the amount of expressed protein present.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. A “nucleic acid array” refers to an array containing nucleic acid probes, such as oligonucleotides or larger portions of genes. The nucleic acid on the array is preferably single stranded. Arrays wherein the probes are oligonucleotides are referred to as “oligonucleotide arrays” or “oligonucleotide chips.” A “microarray,” also referred to herein as a “biochip” or “biological chip” is an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance.

The term “biological sample”, as used herein, refers to a sample obtained from a subject, e.g., a human or from components (e.g., tissues) of a subject. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. A preferred biological sample is e.g. a PBMC sample or a soft tissue.

The term “biomarker” of a disease refers to a gene which is up- or down-regulated in a diseased cell of a subject having a disease or disorder that is caused by or contributed to by a genetic mutation relative to a counterpart normal cell, which gene is sufficiently specific to the diseased cell that it can be used, optionally with other genes, to identify or detect the disease. Generally, a biomarker is a gene that is characteristic of the disease.

A nucleotide sequence is “complementary” to another nucleotide sequence if each of the bases of the two sequences match, i.e., are capable of forming Watson-Crick base pairs. The term “complementary strand” is used herein interchangeably with the term “complement.” The complement of a nucleic acid strand can be the complement of a coding strand or the complement of a non-coding strand.

A “computer readable medium” is any medium that can be used to store data which can be accessed by a computer. Exemplary media include: magnetic storage media, such as a diskettes, hard drives, and magnetic tape; optical storage media such as CD-ROMs; electrical storage media such as RAM and ROM; and hybrids of these media, such as magnetic/optical storage medium.

A “cell carrying a genetic mutation” refers to a cell present in or derived from subjects having a genetic mutation which causes or contributes to a disease or disorder, which cell is a modified form of a normal cell and is generally not present in a subject not having the disease or disorder. A “cell carrying a mutation that causes nonsense-mediated premature protein termination” refers to a cell present in or derived from a subject that carries a genetic mutation not generally present in a comparable wild-type cell, which mutation is a nonsense, frameshift, deletion or other mutation that results in the occurrence of a premature nonsense codon and which thereby results in premature termination of protein translation.

A “cell sample characteristic of a disease or disorder arising from or contributed to by a genetic mutation” or a “tissue sample characteristic of a disease or disorder” refers to a sample of cells, such as a tissue, or a cell line derived from a sample of subject cells, that contains a cell characteristic of the disease or disorder. Such a sample may be e.g. a sample of blood, PBMCs, synovial fluid, synovium, cartilage or bone, or a tumor biopsy.

The term “detecting the level of expression of a gene” refers to any method used to detect the presence of, a threshold amount of or a quantitative measure of the expression of a gene—e.g. by measuring mRNA levels (e.g. by Northern or microarray analysis) or protein (e.g. by detecting the amount of full-length or a truncated polypeptide gene product (e.g. immunologically with an antibody).

The term “derivative” refers to the chemical modification of a compound, e.g., a polypeptide, or a polynucleotide. Chemical modifications of a polynucleotide can include, for example, replacement of hydrogen by an alkyl, acyl, or amino group. A derivative polynucleotide encodes a polypeptide which retains at least one biological or immunological function of the natural molecule. A derivative polypeptide can be one modified by glycosylation, pegylation, or any similar process that retains at least one biological or immunological function of the polypeptide from which it was derived.

A “detection agent of a gene” refers to an agent that can be used to specifically detect a gene or other biological molecule relating to it, e.g., RNA transcribed from the gene and polypeptides encoded by the gene. Exemplary detection agents are nucleic acid probes which hybridize to nucleic acids corresponding to the gene and antibodies.

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids referred to in Any of Tables 1-2 due to the degeneracy of the genetic code.

The term “essentially all the genes of any of Tables 1-2” refers to at least 90%, preferably at least 95% and most preferably at least 98% of the genes of any of Tables 1-2.

The term “expression profile,” which is used interchangeably herein with “gene expression profile” and “finger print” refers to a set of values representing the activity of about 10 or more genes. An expression profile preferably comprises values representing expression levels of at least about 20 genes, preferably at least about 30, 50, 100, 200 or more genes. An expression profile can be a set of values obtained from one or more cells or from a tissue sample, e.g., a clinical sample. An expression profile of a cell characteristic of a particular disease or disorder may refer to a set of values representing mRNA levels of about 10 or more genes in a cell characteristic of the disease or disorder. An “expression profile of a disease or disorder arising from or contributed by a genetic mutation” refers to an expression profile of a cell characteristic of the genetic disease or disorder. Thus, since there are different cells characteristic of the disease or disorder, there may be different expression profiles of the disease or disorder.

The term “gene identification by nonsense-mediated inhibition” (or “GINI”) refers to a method of the invention whereby a gene that carries a genetic mutation which results in nonsense-mediated mRNA decay (NMD) is identified by inhibiting or repressing an NMD pathway and detecting an increasing in expression of the corresponding gene product (i.e. mRNA or polypeptide).

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes which contain certain mismatches, provided that the two strands are still forming a double stranded helix. “Stringent hybridization conditions” refers to hybridization conditions resulting in essentially specific hybridization.

The term “inhibiting nonsense-mediated mRNA decay” refers to any method used to decrease or inhibit the NMD pathway (e.g. in a cell or in vitro).

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the terms “label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens) and the like. The term “fluorescer” refers to a substance or a portion thereof which is capable of exhibiting fluorescence in the detectable range. Particular examples of labels which may be used under the invention include fluorescein, rhodamine, dansyl, umbelliferone, Texas red, luminol, NADPH, alpha-beta-galactosidase and horseradish peroxidase.

The “level of expression of a gene in a cell” refers to the activity of a gene in the cell, which can be indicated by the level of mRNA, as well as pre-mRNA nascent transcript(s), transcript processing intermediates, mature mRNA(s) and degradation products, encoded by the gene in the cell.

The term “library” refers to a collection of biological entities—such as a collection of genes or encoded mRNAs or cDNAs obtained there from.

The term “nonsense-mediated mRNA decay” refers to a pathway in eukaryotic cells that results in the relatively rapid degradation of a message (i.e. mRNA) from a gene carrying a genetic mutation that results in the introduction of a premature nonsense codon (e.g. a nonsense mutation or a frameshift mutation that causes an otherwise out of frame triplet stop codon to be introduced into the reading frame of the encoded polypeptide), or of an improperly transcribed or spliced message which results in a premature stop codon in the resulting mRNA.

The phrase “normalizing expression of a gene” in a diseased cell refers to an action to compensate for the altered expression of the gene in the diseased cell, so that it is essentially expressed at the same level as in the corresponding non diseased cell. For example, where the gene is a mutant gene that causes or contributes to a disease or disorder and is under-expressed in the diseased cell as a result of nonsense-mediated mRNA decay resulting from the genetic mutation, normalization of its expression in the diseased cell refers to treating the diseased cell in such a way that its expression becomes essentially the same as the expression in the counterpart normal cell. “Normalization” preferably brings the level of expression to within approximately a 50% difference in expression, more preferably to within approximately a 25%, and even more preferably 10% difference in expression. The required level of closeness in expression will depend on the particular gene, and can be determined as described herein. The phrase “normalizing gene expression in a diseased cell” refers to an action to normalize the expression of essentially all genes in the diseased cell.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The phrase “nucleic acid corresponding to a gene” refers to a nucleic acid that can be used for detecting the gene, e.g., a nucleic acid which is capable of hybridizing specifically to the gene.

The phrase “nucleic acid sample derived from RNA” refers to one or more nucleic acid molecule, e.g., RNA or DNA, that was synthesized from the RNA, and includes DNA resulting from methods using PCR, e.g., RT-PCR.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases. Databases with individual sequences are described in Methods in Enzymology, ed. Doolittle, supra. Databases include Genbank, EMBL, and DNA Database of Japan (DDBJ).

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. A mismatch in a duplex between a target polynucleotide and an oligonucleotide or olynucleotide means that a pair of nucleotides in the duplex fails to undergo Watson-Crick bonding. In reference to a triplex, the term means that the triplex consists of a perfectly matched duplex and a third strand in which every nucleotide undergoes Hoogsteen or reverse Hoogsteen association with a basepair of the perfectly matched duplex.

The term “phenotype at the cellular level” refers to a phenotype of a disease or disorder that is manifest at the cellular level. For example, cystic fibrosis manifests a disease phenotype at the cellular level (i.e. loss of cAMP-activated chloride channel). In this particular instance, inhibition of NMD results in an improvement in the cellular phenotype—i.e. of the cAMP-activated chloride channel activity) because the stabilized mutant mRNA encodes a truncated polypeptide that retains chloride channel activity), while most other diseases resulting from a nonsense allele-generated dominant negative protein truncation would worsen with inhibition of NMD where the stabilized message encodes a truncated polypeptide which interferes with the normal activity of the full-length protein (e.g. as a dominant-negative protein).

A “plurality” refers to two or more.

As used herein, a nucleic acid or other molecule attached to an array, is referred to as a “probe” or “capture probe.” When an array contains several probes corresponding to one gene, these probes are referred to as “gene-probe set.” A gene-probe set can consist of, e.g., 2 to 10 probes, preferably from 2 to 5 probes and most preferably about 5 probes.

The “profile” of a cell's biological state refers to the levels of various constituents of a cell that are known to change in response to drug treatments and other perturbations of the cell's biological state. Constituents of a cell include levels of RNA, levels of protein abundances, or protein activity levels.

The term “protein” is used interchangeably herein with the terms “peptide” and “polypeptide.”

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

A “subject” can be a mammal, e.g., a human, primate, ovine, bovine, porcine, equine, feline, and canine.

The term “treating” a disease in a subject or “treating” a subject having a disease refers to providing the subject with a pharmaceutical treatment, e.g., the administration of a drug, such that at least one symptom of the disease is decreased. Treating a disease can be preventing the disease, improving the disease or curing the disease.

The phrase “value representing the level of expression of a gene” refers to a raw number which reflects the mRNA level of a particular gene in a cell or biological sample, e.g., obtained from analytical tools for measuring RNA levels.

A “variant” of a polypeptide refers to a polypeptide having the amino acid sequence of the polypeptide, in which one or more amino acid residues are altered. The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR). The term “variant,” when used in the context of a polynucleotide sequence, encompasses a polynucleotide sequence related to that of a gene of interest or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

4.3. Inhibition of Nonsense-Mediated Decay

The invention provides compositions and methods for inhibiting nonsense-mediated mRNA decay (NMD) and/or a component of the NMD pathway in a cell. Exemplary compositions and methods for inhibiting NMD are described in U.S. Pat. Nos. 5,994,119 and 6,048,965, the contents of which are incorporated herein by reference, and in the following sections. The following paragraphs briefly describe the process of nonsense-mediated mRNA decay and provide support for the various points in the pathway and pathway components which may be controlled (e.g. inhibited) in the method of the invention.

Messenger RNAs are monitored for errors that arise during gene expression by a mechanism called RNA surveillance, with the result that most mRNAs that cannot be translated along their full length are rapidly degraded. This ensures that truncated-proteins are seldom made, reducing the accumulation of rogue proteins that might be deleterious. The pathway leading to accelerated mRNA decay is referred as nonsense-meditaed mRNA decay (NMD). The proteins that catalyze steps in NMD in yeast serve two roles, one to monitor errors in gene expression and the other to control the abundance of endogenous wild-type mRNAs as part of the normal repertoire of gene expression. The NMD pathway likely has a direct impact on hundreds of genetic disorders in the human population, where about a quarter of all known mutations are predicted to trigger NMD. For example, base substitutions cause premature polypeptide chain termination whenever a sense condon is changed to a UAA, UAG or UGA stop codon. In AT-rich genomes, multiple stop condons reside in all of the alternate reading frames of virtually every gene. For this reason, most frameshift mutations bring a premature stop condon into register. Because nonsense and frameshift mutations both lead to chain termination, they will be referred to collectively as chain-termination mutations. The mRNAs that contain these mutations will be referred to as nonsense mRNAs.

Most such nonsense mutation-carrying mRNAs are highly unstable because they are degraded by a decay pathway called nonsense-mediated mRNA decay (NMD) (see e.g. Leeds, P. et al. (1991), Genes Dev. 5:2303; and Leeds, P. et al. (1992), Mol. Cell Biol. 12:2165). The process whereby mRNAs are monitored to eliminate those that code for potentially deleterious protein fragments is called RNA surveillance (Pulak, R. and Anderson, P. (1993), Genes Dev. 7:1885). Surveillance occurs in fungi, (Losson, R. and Lacroute, F. (1979), Proc. Natl. Acad. Sci. U.S.A. 76:5134), plants (van Hoof, A. and Green, P. J. (1996), Plant J. 10:415, nematodes, Pulak, R. and Anderson, P. (1993), Genes Dev. 7:1885), and vertebrates (Perlick, H. A. et al. (1996), Proc. Natl. Acad. Sci. U.S.A. 93:10928, and Maquat, L. E. (1995), RNA 1:453). Genes that are required for NMD have been found in Saccharomyces cerevisiae, Caenorhabditis elegans, Mus musculus and Homo sapiens, suggesting the existence of multi-step RNA-decay pathways in these organisms (see e.g. Leeds, P. et al. (1991), Genes Dev. 5:2303; Leeds, P. et al. (1992), Mol. Cell Biol. 12:2165; Pulak, R. and Anderson, P. (1993), Genes Dev. 7:1885; Perlick, H. A. et al. (1996), Proc. Natl. Acad. Sci. U.S.A. 93:10928; Hodgkin, J. et al. (1989), Genetics 123:301; Applequist S. E. et al. (1996), Nucleic Acids Res. 25:814; Sun X. et al. (1998), Proc. Natl. Acad. Sci. U.S.A. 95:10009). Studies of the proteins required for NMD reveal how errors in gene expression is controlled by novel posttranscriptional mechanisms.

NMD is divisible into a sequence of steps, including the recruitment of nonsense mRNAs, premature termination of translation, and possibly late stages leading to decapping and 5′-exonuclease digestion. In S. cerevisiae, three proteins called Upf1p, Upf2p, and Upf3p have been identified that are required to execute these steps (see e.g. Leeds, P. et al. (1992), Mol. Cell Biol. 12:2165; Cui, Y. et al. (1995), Genes Dev. 9:423; He, F. and Jacobson, A. (1995), Genes Dev. 9:437; and Lee, B. S. and Culbertson, M. R. (1995), Proc. Natl. Acad. Sci. U.S.A. 92:10354). All three proteins associate with polyribosomes in the cytoplasm, where they promote the decay of nonsense mRNAs bound in polyribosomes (Atkin, A. L. et al. (1995), Mol. Biol. Cell 6:611; and Atkin, A. L. et al. (1997), J. Biol. Chem. 272:22163).

The biochemical properties of Upf1p suggest the need for a recruitment step to initiate NMD. Four activities can be ascribed to ths protein: ATP-binding, ATP-independent nucleic acid-binding, nucleic acid-dependent ATP hydrolysis, and ATP-dependent 5′→3′ RNA/DNA helicase activity (Czaplinski, K. et al. (1995), RNA 1:610). tRNA nonsense suppressors reduce the efficiency of termination and stabilize nonsense mRNAs, indicating that efficient termination at a premature stop condon is a necessary prerequisite for NMD (Losson, R. and Lacroute, F. (1979), Proc. Natl. Acad. Sci. U.S.A. 76:5134). Two essential termination factors have been identified in S. cerevisiae called eRF1 and eRF3 (see Himmelfarb, H. J. et al. (1985), Mol. Cell. Biol. 5:816; Stansfield, I. et al. (1995), Trends Biochem. Sci. 20:489; Wilson, P. G. and Culbertson, M. R. (1988), J. Mol. Biol. 199:559; and Zhouravleva, G. et al. (1995), EMBO J. 14:4065). Efficient premature termination complex, consisting minimally of Upf1p and the two termination factors, all of which co-purify (Czaplinski, K. et al. (1998), Genes Dev. 12:1665). The association of this complex with polyribosomes occurs irrespective of whether Upf2p or Upf3p are present (Czaplinski, K. et al. (1995), RNA 1:610).

The termination complex catalyzes peptidyl hydrolysis and release of the incomplete polypeptide. The termination factors are released when GTP bound to eRF3 is hydrolyzed to GDP (Stansfield, I. et al. (1995), Trends Biochem. Sci. 20:489). Following GTP hydrolysis and dissociation of the termination factors, Upf1p binds to the mRNA and the ATP is hydrolyzed, which primes the helicase. In order for efficient termination and rapid mRNA decay to occur, the formation of a transient bridge is required between the recruitment and termination complexes, resulting in the assembly of the surveillance complex. This is mediated by a physical interaction between Upf2p and a region encompassing the Cys1/Cys2 domains of Upf1p (He, F. and Jacobson, A. (1995), Genes Dev. 9:437; He, F. et al. (1997), Mol. Cell Biol. 17:1589; and He, F. et al. (1996), RNA 2:153).

4.3.1. Inhibition of NMD with Translational Inhibitors

One method for inhibiting NMD is by use of pharmacological agents that inhibit protein translation. Examples of such drugs are described in Noensie and Dietz ((2001) Nature Biotech 19: 434-439), the contents of which are incorporated herein by reference. This approach is based upon the finding that NMD is generally inhibited by agent that block or inhibit protein translation. Examples of such agents include emetine, anisomycin, cycloheximide, pactamycin, puromycin, gentamicin, neomycin, and paromomycin. Other protein translational inhibitors are known in the art and may be utilized in the method of the invention (see e.g. Leviton (1999) Cancer Invest 17: 87-92 (inhibitors of protein synthesis); and Bertram (2001) Microbiology 147: 255-69 (detailed description of the molecular biology of protein translation)).

4.3.2. Inhibition of NMD with Dominant Negative Polypeptides

Another strategy for inhibition of nonsense-mediated mRNA decay in a test cell is by blocking the pathway by removing or decreasing the biological activity of a necessary component of the pathway—e.g. RENT1 or RENT2. One such method of decreasing the biological activity of a polypeptide is by introducing into the cell a dominant negative mutant which will interfere with the NMD pathway. A dominant negative mutant polypeptide will interact with a molecule with which the polypeptide normally interacts, thereby competing for the molecule, but since it is biologically inactive, it will inhibit the biological activity of the polypeptide. A dominant negative mutant can be created by mutating the substrate-binding domain, the catalytic domain, or a cellular localization domain of the polypeptide. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants. See Herskowitz, Nature (1987) 329:219-222.

An exemplary dominant negative mutant polypetide for use in the invention is a RENT1 mutant encoding an arg to cys mutation at amino acid 843 (R843C) (e.g. SEQ ID NO. 6). Other dominant negative components of the NMD pathway, such as RENT2 dominant negative mutants, can be also be constructed for use in the invention.

4.3.3. Inhibition of NMD with RNAi

Another method for decreasing or blocking gene expression of a component of a nonsense-mediated mRNA decay pathway is by introducing double stranded small interfering RNAs (siRNAs), which mediate sequence specific mRNA degradation. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. Nature 2001; 411(6836):494-8).

To inhibit RENT1 expression, siRNAs composed of the following complementary RNA strands may be used:

| | | |
|---|---|---|
| sense strand - | 5′ GAUGCAGUUCCGCUCCAUUdTdT 3′ | (SEQ ID NO. 1) |
| and | | |
| antisense strand - | 5′ AAUGGAGCGGAACUGCAUCdTdT 3′, | (SEQ ID NO. 2) |
| which form the 19 bp ds siRNA: | GAUGCAGUUCCGCUCCAUUdTdT | |
| | dTdTCUACGUCAAGGCGAGGUAA | |

To inhibit RENT2 expression, we used siRNAs composed of the following complementary RNA strands:

| | | |
|---|---|---|
| sense strand - | 5′ GGCUUUUGUCCCAGCCAUCdTdT 3′ | (SEQ ID NO. 3) |
| and | | |
| antisense strand - | 5′ GAUGGCUGGGACAAAAGCCdTdT 3′, | (SEQ ID NO. 4) |
| which form the 19 bp ds siRNA: | GGCUUUUGUCCCAGCCAUCdTdT | |
| | dTdTCCGAAAACAGGGUCGGUAG | |

In general, the process of RNA interference involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′, 5′ oligoadenylate synthetase (2′,5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

RNAi has been shown to be effective in reducing or eliminating the expression of a target gene in a number of different organisms including Caenorhabditiis elegans (see e.g. Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phophoramidites and thymidine phosphoramidite (Proligo, Germany). Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilized programs designed to predict the secondary structure of a specified single stranded nucleic acid sequence and allow selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions of the invention.

The dsRNA oligonucleotides may be introduced into the cell by transfection with an heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the targeted gene product following sufficient time for turnover of the-endogenous pool after new protein synthesis is repressed, and Northern blot analysis to determine the level of existing target mRNA.

Further compositions, methods and applications of RNAi technology are provided in U.S. Pat. Nos. 6,278,039, 5,723,750 and 5,244,805, which are incorporated herein by reference.

4.3.4. Inhibition of NMD with Antisense

Methods for inhibiting the expression of a gene, e.g. a gene which is a component of the NMD pathway, such that NMD can be inhibited in a test cell using antisense oligonucleotides (e.g. directed against RENT1 and/or RENT2) are known in the art and described in, for example in U.S. Pat. No. 5,814,500, the contents of which are incorporated herein by reference.

In brief, an antisense oligonucleotide is used to decrease the level of expression of an NMD pathway gene by introducing it into a test cell so that antisense molecules which are complementary to at least a portion of the NMD gene or RNA of the gene are targeted. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652: PCT Publication No. WO 88/09810, published Dec. 15, 1988), hybridization-triggered cleavage agents (see, e.g., Krol et al., 1988, BioTechniques 6: 958-976) or intercalating agents (see, e.g., Zon, 1988, Pharm. Res. 5: 539-549).

In a preferred aspect of the invention, an antisense oligonucleotide is provided, preferably as single-stranded DNA. The oligonucleotide may be modified at any position on its structure with constituents generally known in the art. For example, the antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-α-anomeric oligonucleotide. An α-anomeric oligonucleotide forms specific double-stranded hybrids with complementary RNA in which, contrary to the usual α-units, the strands run parallel to each other (Gautier et al., 1987, Nucl. Acids Res. 15:6625-6641).

The oligonucleotide may be conjugated to another molecule, e.g., a peptide, hybridization triggered cross-linking agent transport agent, hybridization-triggered cleavage agent, etc. An antisense molecule can be a “peptide nucleic acid” (PNA). PNA refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

The antisense nucleic acids of the invention comprise a sequence complementary to at least a portion of a target RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

The synthesized antisense oligonucleotides can then be administered to a cell in a controlled manner. For example, the antisense oligonucleotides can be placed in the growth environment of the cell at controlled levels where they may be taken up by the cell. The uptake of the antisense oligonucleotides can be assisted by use of methods well known in the art.

In an alternative embodiment, the antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc.

Antisense therapy for a variety of cancers is in clinical phase and has been discussed extensively in the literature. Reed reviewed antisense therapy directed at the Bcl-2 gene in tumors; gene transfer-mediated overexpression of Bcl-2 in tumor cell lines conferred resistance to many types of cancer drugs. (Reed, J. C., N.C.I (1997) 89:988-990). The potential for clinical development of antisense inhibitors of ras is discussed by Cowsert, L. M., Anti-Cancer Drug Design (1997) 12:359-371. Additional important antisense targets include leukemia (Geurtz, A. M., Anti-Cancer Drug Design (1997) 12:341-358); human C-ref kinase (Monia, B. P., Anti-Cancer Drug Design (1997) 12:327-339); and protein kinase C (McGraw et al., Anti-Cancer Drug Design (1997) 12:315-326.

4.3.5. Inhibition of NMD with Ribozymes

Ribozymes may also be used in the method of the invention for inhibiting the expression of a gene, e.g. a gene which is a component of the NMD pathway, such that NMD is blocked or inhibited in the test cell. The ribozyme is designed to target a component of the NMD pathway—(e.g. directed against RENT1 and/or RENT2 (e.g. SEQ ID Nos. 5, 7 or 8) using techniques which are known in the art and described briefly here below.

Ribozyme molecules designed to catalytically cleave mRNA transcripts can be introduced into, or expressed, in cells to inhibit expression of the gene (see, e.g., Sarver et al., 1990, Science 247:1222-1225 and U.S. Pat. No. 5,093,246). One commonly used ribozyme motif is the hammerhead, for which the substrate sequence requirements are minimal. Design of the hammerhead ribozyme is disclosed in Usman et al., Current Opin. Struct. Biol. (1996) 6:527-533. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16-17; Ojwang et al., Proc. Natl. Acad. Sci. (USA) (1992) 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059-7071. Preparation and use of ribozyme fragments in a hammerhead structure are also described by Koizumi et al., Nucleic Acids Res. (1989) 17:7059-7071. Preparation and use of ribozyme fragments in a hairpin structure are described by Chowrira and Burke, Nucleic Acids Res. (1992) 20:2835. Ribozymes can also be made by rolling transcription as described in Daubendiek and Kool, Nat. Biotechnol. (1997) 15(3):273-277.

Ribozyme molecules designed to catalytically cleave target mRNA transcripts can also be used to prevent translation of target mRNA and expression of target (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to the target gene mRNA, and the well known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of the target mRNA. In addition, ribozymes possess highly specific endoribonuclease activity, which autocatalytically cleaves the target sense mRNA. The present invention extends to ribozyme which hybridize to a sense mRNA encoding a target gene such as a therapeutic drug target candidate gene, thereby hybridizing to the sense mRNA and cleaving it, such that it is no longer capable of being translated to synthesize a functional polypeptide product.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

As in antisense approaches which are also known in the art, the ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes unlike antisense molecules, are catalytic, a lower intracellular concentration is required for efficiency. Ribozyme and RNAi-mediated dsRNAs of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Ribozymes have specific catalytic domains that possess endonuclease activity (Kim and Cech (1987) PNAS USA 84: 8788-92; Gerlach et al. (1987) Nature 328: 802-5; Forster and Symons (1987) Cell 49: 211-20). For example, a large number of ribozymes accelerate phosphoester transfer reactions with a high degree of specificity, often cleaving only one of several phosphoesters in an oligonucleotide substrate (Cech et al. (1981) Cell 27: 487-96; Michel and Westhof (1990) J Mol Biol 216: 585-610; and Reinhold-Hurek and Shub (1992) Nature 357: 173-6). This specificity has been attributed to the requirement that the substrate bind via specific base-pairing interactions to the internal guide sequence (“IGS”) of the ribozyme prior to chemical reaction. U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al. (1991) PNAS USA 88: 10591-95; Sarver et al. (1990) Science 247: 1222-5; Sioud et al. (1992) J Mol Biol 223: 831-5). Recently, it was reported that ribozymes elicited genetic changes in some cells lines to which they were applied; the altered genes included the oncogenes H-ras, c-fos and genes of HIV. Most of these results involved the modification of a target mRNA, based on a specific mutant codon that is cleaved by a specific ribozyme. Several different ribozyme motifs have been described with RNA cleavage activity (Symons (1992) Annu Rev Biochem 61: 641-71).

Hammerhead ribozymes can be reduced in helix 11 to 2 b.p. without loss of activity, but further reduction to 1 b.p. may result in at least a 10-fold reduction in activity. Furthermore, ribozymes designed such that the sequence of “stem-loop II” is 5′GTTTC or 5′GTTTC, where T may be dT or rU, have better than 10% the activity of analogous ribozymes with 4 b.p. in helix II. Such ribozymes are also referred to as “mini-ribozymes”. Furthermore, circular hammerhead ribozymes may be synthesized from linear oligoribonucleotides using T4 RNA ligase. DNA template allows for increased efficiency of their circularization. Such a template may be designed to prevent the precursor from folding into an unsuitable structure, and allows a circular ribozyme as small as 15 nucleotides in length to be efficiently synthesized at concentrations as high as 50 microM in the ligation reaction. The circular products retain their biological activity (see Wang and Ruffner (1998) Nucleic Acids Res; 26: 2502-2504).

Another variable in ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of the homologous sequence can vary in length from a minimum of 5 and preferably 7 to 15 nucleotides in length. One consideration for selecting the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A, C or U) (Perriman et al. (1992) Gene, 113:157-163 and Thompson et al. (1995) Nature Medicine, 1:277-278). The frequency of this dinucleotide occurring in any given RNA is statistically 3 out of 16. Therefore, for a given target messenger RNA of 1000 bases, 187 dinucleotide cleavage sites are statistically probable.

Another consideration when selecting homologous sequences of a target mRNA for incorporation into a ribozyme is the secondary structure of the target mRNA. In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (see Milner et al. (1997) Nat Biotechnol 15: 537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. In addition, RNA-cleaving ribozymes bind to target RNAs via negatively charged regions and cannot “slide” along the RNA chain until they reach the appropriate target sequence. As a consequence, ribozyme-mediated mRNA cleavage occurs via a kinetically unfavorable and repetitive association/dissociation mechanism. In contrast restriction enzymes which bind to DNA via positively charged sites that can “slide” along long stretches of DNA and thereby seek out their target cleavage site are much more kinetically efficient (see Jeltsch et al. (1996) EMBO J 15: 5104-11; and Young (1996) J Mol Biol 264: 440-52). Warashina et al. ((2001) PNAS USA 98: 5572-77) have described improved ribozyme compositions that includes a constitutive transport element (CTE) which recruits RNA helicase (Tang et al. (1997) Science 276: 1412-5; Gruter et al. (1998) Mol Cell 1: 649-59; Braun et al. (199) EMBO J 18: 1953-65; Hodge et al. (1999) EMBO J 18: 5778-88; Kang et al. (1999) Genes Dev. 13: 1126-39); Li et al. (1999) PNAS USA 96: 709-14; Schmitt et al. (1999) EMBO J 18: 4332-47 and Tang et al. (2000) J Biol Chem 275: 32694-32700). The CTE functions as a cytoplasmic transport signal for D-type retroviral RNA (Bray et al. (1994) PNAS USA 91: 1256-60; and Zolotukhin et al. (1994) J Virol 68: 7944-52). The CTE element interacts with a number of RNA helicases in mammalian cells such as hDbp5 and RHA (see (Tang et al. (1997) Science 276: 1412-5; Gruter et al. (1998) Mol Cell 1: 649-59; Braun et al. (199) EMBO J 18: 1953-65; Hodge et al. (1999) EMBO J 18: 5778-88; Kang et al. (1999) Genes Dev. 13: 1126-39); Li et al. (1999) PNAS USA 96: 709-14; Schmitt et al. (1999) EMBO J 18: 4332-47 and Tang et al. (2000) J Biol Chem 275: 32694-32700). Endogenous RNA helicases may thereby be recruited to the recombinant ribozymes of the invention, or may be supplied heterologously. An exemplary CTE sequence for incorporation into the design of the ribozyme is: ttcaccaaga gctgtgacac caagaactgt gtcaccaaaa tctgtgatac ctagagctat gatacctaga gctgtgtcac caagagctgt gtcaccaaga gctgtgacac caagagctgt gataccaaga gctgtgacac caagagctgt gatacctaga gctgtgtcac caagagctgt gacaccaaga gctgtgatac ctagagctgt gtcaccaaga gctgtgacct agagctgtg which is GenBank Accession No. AF260329 (Zolotukhin et al. (2001) J. Virol. 75: 5567-5575). Furthermore, Tip-associated protein functions in the interaction of hDbp5 with CTE (Kang et al. (1999) Genes Dev. 13: 1126-39) and cells devoid of Tip-associated protein may be used to modify the ribozyme activity and test specificity of target repression and biological effects (see Warashina et al. (2001) PNAS USA 98: 5572-77). Ribozymes incorporating such CTE sequences were found to have improved properties, including the ability to cleave sequences refractory because of RNA secondary structure and apparently improved kinetics. Without limiting the CTE-incorporating ribozymes to a single mode of action, it is likely that the element recruits an endogenous cellular RNA helicase and unwinds inhibitory structures and that it may further facilitate “sliding” of the RNA helicase along the target RNA (see Warashina et al. (2001) PNAS USA 98: 5572-77).

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in selected gene-targeted ribozymes is simply a matter of preparing and testing a given sequence, and is a routinely practiced “screening” method known to those of skill in the art.

Further compositions, methods and applications of ribozyme technology are provided in U.S. Pat. Nos. 6,281,375, 6,277,565, 6,274,342, 6,274,339, 6,271,440, and 6,271,436, the contents of which are incorporated herein by reference.

4.3.6. Other Methods for Inhibiting NMD

Triplex Formation

Gene expression can be reduced by targeting deoxyribonucleotide sequences complementary to the regulatory region of the target gene (i.e., the gene promoter and/or enhancers) to form triple helical structures that prevent transcription of the gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

Aptamers

In a further embodiment, RNA aptamers can be introduced into or expressed in a cell. RNA aptamers are specific RNA ligands for proteins, such as for Tat and Rev RNA (Good et al., 1997, Gene Therapy 4: 45-54) that can specifically inhibit their translation.

4.4. Preparation of Cell Samples and mRNA

In general, the GINI methodology operates optimally when the relevant transcript is normally expressed in the tissue type from which the sample cell or cell population is derived. This ensures that the nonsense-carrying mutant transcript will be expressed in the control (untreated) cell population and be subject to a detectable increase in abundance following treatment to inhibit nonsense mediated decay. While detectable expression in the source tissue is optimal, it should be noted that even illegitimate transcripts appear to be substrates for NMD (see e.g. Freddi et al. (2000) Am J Med Genet 90: 398-406; and Bateman et al. (1999) Hum Matat 13: 311-17). Accordingly, GINI may be applied to the detection of nonsense alleles even in cases where the transcript is not functionally important in the experimental cell or cell population (e.g. a cell sample or cell line derived from a human subject).

In one embodiment, one or more cells from the subject to be tested are obtained and RNA is isolated from the cells. In a preferred embodiment, PBMCs, synovial fluid, synovium or cartilage are obtained from the subject according to methods known in the art. Examples of such methods are set forth in the Examples and is discussed by Kim, C. H. et al. (J. Virol. 66:3879-3882 (1992)); Biswas, B. et al. (Annals NY Acad. Sci. 590:582-583 (1990)); Biswas, B. et al. (J. Clin. Microbiol. 29:2228-2233 (1991)). When obtaining the cells, it is preferable to obtain a sample containing predominantly cells of the desired type, e.g., a sample of cells in which at least about 50%, preferably at least about 60%, even more preferably at least about 70%, 80% and even more preferably, at least about 90% of the cells are of the desired type. A higher percentage of cells of the desired type is preferable, since such a sample is more likely to provide clear gene expression data.

It is also possible to obtain a cell sample from a subject, and then to enrich it for a desired cell type. For example, PBMCs can be isolated from blood as described herein. Counter-flow centrifugation (elutriation) can also be used to enrich for various cell types, such as T cells, B cells and monocytes, from PBMCs. Cells can also be isolated from other cells using a variety of techniques, such as isolation with an antibody binding to an epitope on the cell surface of the desired cell type. Another method that can be used includes negative selection using antibodies to cell surface markers to selectively enrich for a specific cell type without activating the cell by receptor engagement. Where the desired cells are in a solid tissue, particular cells can be dissected out, e.g., by microdissection. Exemplary cells that one may want to enrich for include monocytes, macrophages, T and B cells, osteocytes, osteoblasts, osteoclasts, chondrocytes, fibroblasts, neutrophils, endothelial cells and other cartilage cells.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., those described in the Examples or guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in sequences of interest, such as those of genes characteristic of a genetic mutation that causes nonsense mediated mRNA decay and which is associated with or causes a human disease or disorder. Enrichment can be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, can further be amplified. Such amplification is particularly important when using RNA from a single or a few cells. A variety of amplification methods are suitable for use in the methods of the invention, including, e.g., PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); nucleic acid based sequence amplification (NASBA) and transcription amplification (see, e.g., Kwoh et al., Proc. Natl. Acad. Sci. USA 86, 1173 (1989)). For PCR technology, see, e.g., PCR Technology: Principles and Applications for DNA Amplification (ed. H. A. Erlich, Freeman Press, N.Y., N.Y., 1992); PCR Protocols: A Guide to Methods and applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202. Methods of amplification are described, e.g., in Ohyama et al. (2000) BioTechniques 29:530; Luo et al. (1999) Nat. Med. 5, 117; Hegde et al. (2000) BioTechniques 29:548; Kacharmina et al. (1999) Meth. Enzymol. 303:3; Livesey et al. (2000) Curr. Biol. 10:301; Spirin et al. (1999) Invest. Ophtalmol. Vis. Sci. 40:3108; and Sakai et al. (2000) Anal. Biochem. 287:32. RNA amplification and cDNA synthesis can also be conducted in cells in situ (see, e.g., Eberwine et al. (1992) PNAS 89:3010).

One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. A high density array may then include probes specific to the internal standard for quantification of the amplified nucleic acid.

One preferred internal standard is a synthetic AW106 cRNA. The AW106 ERNA is combined with RNA isolated from the sample according to standard techniques known to those of skilled in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

In a preferred embodiment, a sample mRNA is reverse transcribed with a reverse transcriptase and a primer consisting of oligo(dT) and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template Successive rounds of transcription from each single cDNA template results in amplified RNA. Methods of in vitro polymerization are well known to those of skill in the art (see, e.g., Sambrook, (supra) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts). Moreover, Eberwine et al. Proc. Natl. Acad. Sci. USA, 89: 3010-3014 provide a protocol that uses two rounds of amplification via in vitro transcription to achieve greater than 106 fold amplification of the original starting material, thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

4.5. Analysis of mRNA Transcripts

In certain embodiments, it is sufficient to determine the expression of one or only a few genes, as opposed to hundreds or thousands of genes. Although microarrays can be used in these embodiments, various other methods of detection of gene expression are available. This section describes a few exemplary methods for detecting and quantifying mRNA or polypeptide encoded thereby. Where the first step of the methods includes isolation of mRNA from cells, this step can be conducted as described above. Labeling of one or more nucleic acids can be performed as described above.

In one embodiment, mRNA obtained form a sample is reverse transcribed into a first cDNA strand and subjected to PCR, e.g., RT-PCR. House keeping genes, or other genes whose expression does not vary can be used as internal controls and controls across experiments. Following the PCR reaction, the amplified products can be separated by electrophoresis and detected. By using quantitative PCR, the level of amplified product will correlate with the level of RNA that was present in the sample. The amplified samples can also be separated on a agarose or polyacrylamide gel, transferred onto a filter, and the filter hybridized with a probe specific for the gene of interest. Numerous samples can be analyzed simultaneously by conducting parallel PCR amplification, e.g., by multiplex PCR.

A quantitative PCR technique that can be used is based on the use of TaqMan™ probes. Specific sequence detection occurs by amplification of target sequences in the PE Applied Biosystems 7700 Sequence Detection System in the presence of an oligonucleotide probe labeled at the 5′ and 3′ ends with a reporter and quencher fluorescent dye, respectively (FQ probe), which anneals between the two PCR primers. Only specific product will be detected when the probe is bound between the primers. As PCR amplification proceeds, the 5′-nuclease activity of Taq polymerase initially cleaves the reporter dye from the probe. The signal generated when the reporter dye is physically separated from the quencher dye is detected by measuring the signal with an attached CCD camera. Each signal generated equals one probe cleaved which corresponds to amplification of one target strand. PCR reactions may be set up using the PE Applied Biosystem TaqMan PCR Core Reagent Kit according to the instructions supplied. This technique is further described, e.g., in U.S. Pat. No. 6,326,462.

In another embodiment, mRNA levels is determined by dotblot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples can be analyzed simultaneously, since a blot can comprise multiple spots of RNA. Hybridization is detected using a method that depends on the type of label of the probe. In another dotblot method, one or more probes of one or more genes which are up- or down-regulated in R.A. are attached to a membrane, and the membrane is incubated with labeled nucleic acids obtained from and optionally derived from RNA of a cell or tissue of a subject. Such a dotblot is essentially an array comprising fewer probes than a microarray.

“Dot blot” hybridization gained wide-spread use, and many versions were developed (see, e.g., M. L. M. Anderson and B. D. Young, in Nucleic Acid Hybridization—A Practical Approach, B. D. Hames and S. J. Higgins, Eds., IRL Press, Washington D.C., Chapter 4, pp. 73-111, 1985).

Another format, the so-called “sandwich” hybridization, involves covalently attaching oligonucleotide probes to a solid support and using them to capture and detect multiple nucleic acid targets (see, e.g., M. Ranki et al., Gene, 21, pp. 77-85, 1983; A. M. Palva, T. M. Ranki, and H. E. Soderlund, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. 6 Nucleic Acid Res. 11, p. 3543, 1979; and B. J. Connor et al., 80 Proc. Natl. Acad. Sci. USA pp. 278-282, 1983). Multiplex versions of these formats are called “reverse dot blots.”

mRNA levels can also be determined by Northern blots. Specific amounts of RNA are separated by gel electrophoresis and transferred onto a filter which is then hybridized with a probe corresponding to the gene of interest. This method, although more burdensome when numerous samples and genes are to be analyzed provides the advantage of being very accurate.

A preferred method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, provides quantitative information about the relative expression of such genes, permits ready comparison of gene expression of genes in two cells, and yields sequence information that can be used to identify the detected genes. Thus far, SAGE methodology has proved itself to reliably detect expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388).

Techniques for producing and probing nucleic acids are further described, for example, in Sambrook et al., “Molecular Cloning: A Laboratory Manual” (New York, Cold Spring Harbor Laboratory, 1989).

Alternatively, the level of expression of one or more genes which are up- or down-regulated in R.A. is determined by in situ hybridization. In one embodiment, a tissue sample is obtained from a subject, the tissue sample is sliced, and in situ hybridization is performed according to methods known in the art, to determine the level of expression of the genes of interest.

In other methods, the level of expression of a gene is detected by measuring the level of protein encoded by the gene. This can be done, e.g., by immunoprecipitation, ELISA, or immunohistochemistry using an agent, e.g., an antibody, that specifically detects the protein encoded by the gene. Other techniques include Western blot analysis. Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In the case of polypeptides which are secreted from cells, the level of expression of these polypeptides can be measured in biological fluids.

In preferred embodiments, mRNA levels are detected and/or measured by microarray analysis as described in detail in the following sections.

4.5.1. Analysis of mRNA by Microarray

Generally, determining expression profiles with arrays involves the following steps: (a) obtaining a mRNA sample from a subject and preparing labeled nucleic acids therefrom (the “target nucleic acids” or “targets”); (b) contacting the target nucleic acids with the array under conditions sufficient for target nucleic acids to bind with corresponding probes on the array, e.g. by hybridization or specific binding; (c) optionally removing unbound targets from the array; (d) detecting bound targets, and (e) analyzing the results. As used herein, “nucleic acid probes” or “probes” are nucleic acids attached to the array, whereas “target nucleic acids” are nucleic acids that are hybridized to the array. Each of these steps is described in more detail below.

4.5.2. Labeling of the Nucleic Acids to be Analyzed

Generally, the target molecules will be labeled to permit detection of hybridization of target molecules to a microarray. By “labeled” is meant that the probe comprises a member of a signal producing system and is thus detectable, either directly or through combined action with one or more additional members of a signal producing system. Examples of directly detectable labels include isotopic and fluorescent moieties incorporated into, usually covalently bonded to, a moiety of the probe, such as a nucleotide monomeric unit, e.g. dNMP of the primer, or a photoactive or chemically active derivative of a detectable label which can be bound to a functional moiety of the probe molecule.

Nucleic acids can be labeled after or during enrichment and/or amplification of RNAs. For example, labeled cDNA can be prepared from mRNA by oligo dT-primed or random-primed reverse transcription, both of which are well known in the art (see, e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Expression monitoring by hybridization to high-density oligonucleotide arrays, Nature Biotech. 14:1675). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

In one embodiment, labeled cDNA is synthesized by incubating a mixture containing RNA and 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript.™.II, LTI Inc.) at 42° C. for 60 min.

Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy2, Cy3, Cy3.5, Cy5, Cy5.5, Cy7, Fluor X, macrocyclic chelates of lanthamide ions, e.g. quantum dye™, fluorescent energy transfer dyes, such as thiazole orange-ethidium heterodimer, TOTAB, dansyl, etc. Individual fluorescent compounds which have functionalities for linking to an element desirably detected in an apparatus or assay of the invention, or which can be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2- -methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)furanone. (see, e.g., Kricka, 1992, Nonisotopic DNA Probe Techniques, Academic Press San Diego, Calif.). Many fluorescent tags are commercially available from SIGMA chemical company (Saint Louis, Mo.), Amersham, Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill.

Chemiluminescent labels include luciferin and 2,3-dihydrophthalazinediones, e.g., luminol.

Isotopic moieties or labels of interest include ³²P, ³³P, ³⁵S, ¹²⁵I, ²H, ¹⁴C, and the like (see Zhao et al., 1995, High density cDNA filter analysis: a novel approach for large-scale, quantitative analysis of gene expression, Gene 156:207; Pietu et al., 1996, Novel gene transcripts preferentially expressed in human muscles revealed by quantitative hybridization of a high density cDNA array, Genome Res. 6:492).

Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody and the like.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

In some cases, hybridized target nucleic acids may be labeled following hybridization. For example, where biotin labeled dNTPs are used in, e.g., amplification or transcription, streptavidin linked reporter groups may be used to label hybridized complexes.

In other embodiments, the target nucleic acid is not labeled. In this case, hybridization can be determined, e.g., by plasmon resonance, as described, e.g., in Thiel et al. (1997) Anal. Chem. 69:4948.

In one embodiment, a plurality (e.g., 2, 3, 4, 5 or more) of sets of target nucleic acids are labeled and used in one hybridization reaction (“multiplex” analysis). For example, one set of nucleic acids may correspond to RNA from one cell or tissue sample and another set of nucleic acids may correspond to RNA from another cell or tissue sample. The plurality of sets of nucleic acids can be labeled with different labels, e.g., different fluorescent labels which have distinct emission spectra so that they can be distinguished. The sets can then be mixed and hybridized simultaneously to one microarray.

For example, the two different cells can be a diseased cell of a patient having R.A. and a counterpart normal cell. Alternatively, the two different cells can be a diseased cell of a patient having R.A. and a diseased cell of a patient suspected of having R.A. In another embodiment, one biological sample is exposed to a drug and another biological sample of the same type is not exposed to the drug. The cDNA derived from each of the two cell types are differently labeled so that they can be distinguished. In one embodiment, for example, cDNA from a diseased cell is synthesized using a fluorescein-labeled dNTP, and cDNA from a second cell, i.e., the normal cell, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the diseased cell will fluoresce green when the fluorophore is stimulated and the cDNA from the cell of a subject suspected of having R.A. will fluoresce red. As a result, if the two cells are essentially the same, the particular mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores (and appear brown in combination). In contrast, if the two cells are different, the ratio of green to red fluorescence will be different.

The use of a two-color fluorescence labeling and detection scheme to define alterations in gene expression has been described, e.g., in Shena et al., 1995, Quantitative monitoring of gene expression patterns with a complementary DNA microarray, Science 270:467-470. An advantage of using cDNA labeled with two different fluorophores is that a direct and internally controlled comparison of the mRNA levels corresponding to each arrayed gene in two cell states can be made, and variations due to minor differences in experimental conditions (e.g, hybridization conditions) will not affect subsequent analyses.

Examples of distinguishable labels for use when hybridizing a plurality of target nucleic acids to one array are well known in the art and include: two or more different emission wavelength fluorescent dyes, like Cy3 and Cy5, combination of fluorescent proteins and dyes, like phicoerythrin and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Further, it is preferable in order to reduce experimental error to reverse the fluorescent labels in two-color differential hybridization experiments to reduce biases peculiar to individual genes or array spot locations. In other words, it is preferable to first measure gene expression with one labeling (e.g., labeling nucleic acid from a first cell with a first fluorochrome and nucleic acid from a second cell with a second fluorochrome) of the mRNA from the two cells being measured, and then to measure gene expression from the two cells with reversed labeling (e.g., labeling nucleic acid from the first cell with the second fluorochrome and nucleic acid from the second cell with the first fluorochrome). Multiple measurements over exposure levels and perturbation control parameter levels provide additional experimental error control.

The quality of labeled nucleic acids can be evaluated prior to hybridization to an array. For example, a sample of the labeled nucleic acids can be hybridized to probes derived from the 5′, middle and 3′ portions of genes known to be or suspected to be present in the nucleic acid sample. This will be indicative as to whether the labeled nucleic acids are full length nucleic acids or whether they are degraded. In one embodiment, the GeneChip® Test3 Array from Affymetrix (Santa Clara, Calif.) can be used for that purpose. This array contains probes representing a subset of characterized genes from several organisms including mammals. Thus, the quality of a labeled nucleic acid sample can be determined by hybridization of a fraction of the sample to an array, such as the GeneChip® Test3 Array from Affymetrix (Santa Clara, Calif.).

4.5.3. Gene Arrays

Preferred arrays, e.g., microarrays, for use according to the invention include one or more probes of genes which are candidate genes for being affected by a genetic mutation that causes or contributes to a disease or disorder. Exemplary arrays include one or more genes listed in either of Tables 1-2 or one or more genes characteristic of or associated with a disease or disorder. For example, where the disease or disorder is a cancer, exemplary arrays would contain one or more oncogene or tumor suppressor genes such as: met, Her-2/neu, src, ras, and other oncogenes as well as p53, RIZ, ING, NF1, NF2 and other tumor suppressor genes. Other suitable genes to be included in the arrays of the invention include gene sequences associated with cancers such as unique gene fusions arising from chromosomal translocations such as those found in renal neoplasms including the ASPL-TFE3 fusion gene (Argani et al. (2001) Am J Pathol 159: 179-92) and the PRCC-TFE3 fusion gene (Weterman et al. (2001) Oncogene 20: 1414-24). Still other preferred arrays contain one or more genes representing background to inhibition of nonsense-mediated mRNA decay: early growth response protein 1, hormone receptor (growth factor-inducible nuclear protein N10), putative DNA-binding protein A20, early growth response protein 2, p55-c-fos proto-oncogene, major histocompatibility complex enhancer-binding protein MAD3, gem GTPase, transcription factor RELB, spermidine/spermine N1-acetyltransferase, thyroid hormone receptor, alpha; DNA-damage-inducible transcript 1, dual-specificity protein phosphatase PAC-1, interferon regulatory factor 1, interleukin 1, alpha, V-abl Abelson murine leukemia viral oncogene homolog 2, DEC1, diphtheria toxin receptor, early growth response protein 3, putative transmembrane protein NMA, peptidyl-prolyl cis-trans isomerase, IAP homolog C MIHC, thyroid receptor interactor TRIP9, natural killer cells protein 4 precursor and small inducible cytokine A2. These genes are also represented by GenBank Accession Nos.: X52541, D49728, M59465, J04076, M69043, U10550, M83221, U40369, M24898, L24498, L11329, X14454, M28983, M35296, AB004066, M60278, X63741, U23070, M80254, U37546, L40407, M59807 and M26683.

The array may comprise probes corresponding to at least 10, preferably at least 20, at least 50, at least 100 or at least 1000 genes. The array may comprise probes corresponding to about 10%, 20%, 50%, 70%, 90% or 95% of the genes listed in any of Tables 1-2 or other gene. The array may comprise probes corresponding to about 10%, 20%, 50%, 70%, 90% or 95% of the genes listed in any of Tables 1-2 or other gene whose expression is at least 2 fold, preferably at least 3 fold, more preferably at least 4 fold, 5 fold, 7 fold and most preferably at least about 10 fold higher in cells in which nonsense-mediated mRNA decay is inhibited relative to normal counterpart cells in which no action to inhibit NMD has been taken. One exemplary preferred array that can be used is the array used and described in the Examples.

There can be one or more than one probe corresponding to each gene on a microarray. For example, a microarray may contain from 2 to 20 probes corresponding to one gene and preferably about 5 to 10. The probes may correspond to the full length RNA sequence or complement thereof of genes characteristic of candidate disease genes, or they may correspond to a portion thereof, which portion is of sufficient length for permitting specific hybridization. Such probes may comprise from about 50 nucleotides to about 100, 200, 500, or 1000 nucleotides or more than 1000 nucleotides. As further described herein, microarrays may contain oligonucleotide probes, consisting of about 10 to 50 nucleotides, preferably about 15 to 30 nucleotides and even more preferably 20-25 nucleotides. The probes are preferably single stranded. The probe will have sufficient complementarity to its target to provide for the desired level of sequence specific hybridization (see below).

Typically, the arrays used in the present invention will have a site density of greater than 100 different probes per cm². Preferably, the arrays will have a site density of greater than 500/cm², more preferably greater than about 1000/cm², and most preferably, greater than about 10,000/cm². Preferably, the arrays will have more than 100 different probes on a single substrate, more preferably greater than about 1000 different probes still more preferably, greater than about 10,000 different probes and most preferably, greater than 100,000 different probes on a single substrate.

Microarrays can be prepared by methods known in the art, as described below, or they can be custom made by companies, e.g., Affymetrix (Santa Clara, Calif.).

Generally, two types of microarrays can be used. These two types are referred to as “synthesis” and “delivery.” In the synthesis type, a microarray is prepared in a step-wise fashion by the in situ synthesis of nucleic acids from nucleotides. With each round of synthesis, nucleotides are added to growing chains until the desired length is achieved. In the delivery type of microarray, preprepared nucleic acids are deposited onto known locations using a variety of delivery technologies. Numerous articles describe the different microarray technologies, e.g., Shena et al. (1998) Tibtech 16: 301; Duggan et al. (1999) Nat. Genet. 21:10; Bowtell et al. (1999) Nat. Genet. 21: 25.

One novel synthesis technology is that developed by Affymetrix (Santa Clara, Calif.), which combines photolithography technology with DNA synthetic chemistry to enable high density oligonucleotide microarray manufacture. Such chips contain up to 400,000 groups of oligonucleotides in an area of about 1.6 cm². Oligonucleotides are anchored at the 3′ end thereby maximizing the availability of single-stranded nucleic acid for hybridization. Generally such chips, referred to as “GeneChips®” contain several oligonucleotides of a particular gene, e.g., between 15-20, such as 16 oligonucleotides. Since Affymetrix (Santa Clara, Calif.) sells custom made microarrays, microarrays containing genes which are up- or down-regulated in R.A. can be ordered for purchase from Affymetrix (Santa Clara, Calif.).

Microarrays can also be prepared by mechanical microspotting, e.g., those commercialized at Synteni (Fremont, Calif.). According to these methods, small quantities of nucleic acids are printed onto solid surfaces. Microspotted arrays prepared at Synteni contain as many as 10,000 groups of cDNA in an area of about 3.6 cm².

A third group of microarray technologies consist in the “drop-on-demand” delivery approaches, the most advanced of which are the ink-jetting technologies, which utilize piezoelectric and other forms of propulsion to transfer nucleic acids from miniature nozzles to solid surfaces. Inkjet technologies is developed at several centers including Incyte Pharmaceuticals (Palo Alto, Calif.) and Protogene (Palo Alto, Calif.). This technology results in a density of 10,000 spots per cm². See also, Hughes et al. (2001) Nat. Biotechn. 19:342.

Arrays preferably include control and reference nucleic acids. Control nucleic acids are nucleic acids which serve to indicate that the hybridization was effective. For example, all Affymetrix (Santa Clara, Calif.) expression arrays contain sets of probes for several prokaryotic genes, e.g., bioB, bioC and bioD from biotin synthesis of E. coli and cre from P1 bacteriophage. Hybridization to these arrays is conducted in the presence of a mixture of these genes or portions thereof, such as the mix provided by Affymetrix (Santa Clara, Calif.) to that effect (Part Number 900299), to thereby confirm that the hybridization was effective. Control nucleic acids included with the target nucleic acids can also be mRNA synthesized from cDNA clones by in vitro transcription. Other control genes that may be included in arrays are polyA controls, such as dap, lys, phe, thr, and trp (which are included on Affymetrix GeneChips®)

Reference nucleic acids allow the normalization of results from one experiment to another, and to compare multiple experiments on a quantitative level. Exemplary reference nucleic acids include housekeeping genes of known expression levels, e.g., GAPDH, hexokinase and actin.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Arrays may also contain probes that hybridize to more than one allele of a gene. For example the array can contain one probe that recognizes allele 1 and another probe that recognizes allele 2 of a particular gene.

Microarrays can be prepared as follows. In one embodiment, an array of oligonucleotides is synthesized on a solid support. Exemplary solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips,” or as very large scale immobilized polymer arrays (“VLSIPS™” arrays) can include millions of defined probe regions on a substrate having an area of about 1 cm to several cm², thereby incorporating sets of from a few to millions of probes (see, e.g., U.S. Pat. No. 5,631,734).

The construction of solid phase nucleic acid arrays to detect target nucleic acids is well described in the literature. See, Fodor et al. (1991) Science, 251: 767-777; Sheldon et al. (1993) Clinical Chemistry 39(4): 718-719; Kozal et al. (1996) Nature Medicine 2(7): 753-759 and Hubbell U.S. Pat. No. 5,571,639; Pinkel et al. PCT/US95/16155 (WO 96/17958); U.S. Pat. Nos. 5,677,195; 5,624,711; 5,599,695; 5,451,683; 5,424,186; 5,412,087; 5,384,261; 5,252,743 and 5,143,854; PCT Patent Publication Nos. 92/10092 and 93/09668; and PCT WO 97/10365. In brief, a combinatorial strategy allows for the synthesis of arrays containing a large number of probes using a minimal number of synthetic steps. For instance, it is possible to synthesize and attach all possible DNA 8 mer oligonucleotides (48, or 65,536 possible combinations) using only 32 chemical synthetic steps. In general, VLSIPS™ procedures provide a method of producing 4n different oligonucleotide probes on an array using only 4n synthetic steps (see, e.g., U.S. Pat. No. 5,631,7345; 143,854 and PCT Patent Publication Nos. WO 90/15070; WO 95/11995 and WO 92/10092).

Light-directed combinatorial synthesis of oligonucleotide arrays on a glass surface can be performed with automated phosphoramidite chemistry and chip masking techniques similar to photoresist technologies in the computer chip industry. Typically, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

Algorithms for design of masks to reduce the number of synthesis cycles are described by Hubbel et al., U.S. Pat. No. 5,571,639 and U.S. Pat. No. 5,593,839. A computer system may be used to select nucleic acid probes on the substrate and design the layout of the array as described in U.S. Pat. No. 5,571,639.

Another method for synthesizing high density arrays is described in U.S. Pat. No. 6,083,697. This method utilizes a novel chemical amplification process using a catalyst system which is initiated by radiation to assist in the synthesis the polymer sequences. Such methods include the use of photosensitive compounds which act as catalysts to chemically alter the synthesis intermediates in a manner to promote formation of polymer sequences. Such photosensitive compounds include what are generally referred to as radiation-activated catalysts (RACs), and more specifically photo activated catalysts (PACs). The RACs can by themselves chemically alter the synthesis intermediate or they can activate an autocatalytic compound which chemically alters the synthesis intermediate in a manner to allow the synthesis intermediate to chemically combine with a later added synthesis intermediate or other compound.

Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id. and Pease et al., EP 728,520.

cDNA probes can be prepared according to methods known in the art and further described herein, e.g., reverse-transcription PCR (RT-PCR) of RNA using sequence specific primers. Oligonucleotide probes can be synthesized chemically. Sequences of the genes or cDNA from which probes are made can be obtained, e.g., from GenBank, other public databases or publications.

Nucleic acid probes can be natural nucleic acids, chemically modified nucleic acids, e.g., composed of nucleotide analogs, as long as they have activated hydroxyl groups compatible with the linking chemistry. The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, which results in a free 5′ end.

Oligonucleotides of an array can be synthesized using a 96 well automated multiplex oligonucleotide synthesizer (A.M.O.S.) that is capable of making thousands of oligonucleotides (Lashkari et al. (1995) PNAS 93: 7912) can be used.

It will be appreciated that oligonucleotide design is influenced by the intended application. For example, it may be desirable to have similar melting temperatures for all of the probes. Accordingly, the length of the probes are adjusted so that the melting temperatures for all of the probes on the array are closely similar (it will be appreciated that different lengths for different probes may be needed to achieve a particular T[m] where different probes have different GC contents). Although melting temperature is a primary consideration in probe design, other factors are optionally used to further adjust probe construction, such as selecting against primer self-complementarity and the like.

Arrays, e.g., microarrays, may conveniently be stored following fabrication or purchase for use at a later time. Under appropriate conditions, the subject arrays are capable of being stored for at least about 6 months and may be stored for up to one year or longer. Arrays are generally stored at temperatures between about −20° C. to room temperature, where the arrays are preferably sealed in a plastic container, e.g. bag, and shielded from light.

4.5.4. Hybridization of the Target Nucleic Acids to the Microarray

The next step is to contact the target nucleic acids with the array under conditions sufficient for binding between the target nucleic acids and the probes of the array. In a preferred embodiment, the target nucleic acids will be contacted with the array under conditions sufficient for hybridization to occur between the target nucleic acids and probes on the microarray, where the hybridization conditions will be selected in order to provide for the desired level of hybridization specificity.

Contact of the array and target nucleic acids involves contacting the array with an aqueous medium comprising the target nucleic acids. Contact may be achieved in a variety of different ways depending on specific configuration of the array. For example, where the array simply comprises the pattern of size separated probes on the surface of a “plate-like” rigid substrate, contact may be accomplished by simply placing the array in a container comprising the target nucleic acid solution, such as a polyethylene bag, and the like. In other embodiments where the array is entrapped in a separation media bounded by two rigid plates, the opportunity exists to deliver the target nucleic acids via electrophoretic means. Alternatively, where the array is incorporated into a biochip device having fluid entry and exit ports, the target nucleic acid solution can be introduced into the chamber in which the pattern of target molecules is presented through the entry port, where fluid introduction could be performed manually or with an automated device. In multiwell embodiments, the target nucleic acid solution will be introduced in the reaction chamber comprising the array, either manually, e.g. with a pipette, or with an automated fluid handling device.

Contact of the target nucleic acid solution and the probes will be maintained for a sufficient period of time for binding between the target and the probe to occur. Although dependent on the nature of the probe and target, contact will generally be maintained for a period of time ranging from about 10 min to 24 hrs, usually from about 30 min to 12 hrs and more usually from about 1 hr to 6 hrs.

When using commercially available microarrays, adequate hybridization conditions are provided by the manufacturer. When using non-commercial microarrays, adequate hybridization conditions can be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls.

Hybridization is carried out in conditions permitting essentially specific hybridization. The length of the probe and GC content will determine the Tm of the hybrid, and thus the hybridization conditions necessary for obtaining specific hybridization of the probe to the template nucleic acid. These factors are well known to a person of skill in the art, and can also be tested in assays. An extensive guide to the hybridization of nucleic acids is found in Tijssen (1993), “Laboratory Techniques in biochemistry and molecular biology-hybridization with nucleic acid probes.” Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5 degrees. See Tibanyenda, N. et al., Eur. J. Biochem. 139:19 (1984) and Ebel, S. et al., Biochem. 31:12083 (1992). More importantly, it is understood that as the length of the homology region increases, the effect of a single base mismatch on overall duplex stability decreases.

Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York provide a basic guide to nucleic acid hybridization.

Certain microarrays are of “active” nature, i.e., they provide independent electronic control over all aspects of the hybridization reaction (or any other affinity reaction) occurring at each specific microlocation. These devices provide a new mechanism for affecting hybridization reactions which is called electronic stringency control (ESC). Such active devices can electronically produce “different stringency conditions” at each microlocation. Thus, all hybridizations can be carried out optimally in the same bulk solution. These arrays are described in U.S. Pat. No. 6,051,380 by Sosnowski et al.

In a preferred embodiment, background signal is reduced by the use of a detergent (e.g, C-TAB) or a blocking reagent (e.g., sperm DNA, cot-1 DNA, etc.) during the hybridization to reduce non-specific binding. In a particularly preferred (embodiment, the hybridization is performed in the presence of about 0.5 mg/ml DNA (e.g., herring sperm DNA). The use of blocking agents in hybridization is well known to those of skill in the art (see, e.g., Chapter 8 in Laboratory Techniques in Biochemistry and Molecular Biology, Vol. 24: Hybridization With Nucleic Acid Probes, P. Tijssen, ed. Elsevier, N.Y., (1993)).

The method may or may not further comprise a non-bound label removal step prior to the detection step, depending on the particular label employed on the target nucleic acid. For example, in certain assay formats (e.g., “homogenous assay formats”) a detectable signal is only generated upon specific binding of target to probe. As such, in these assay formats, the hybridization pattern may be detected without a non-bound label removal step. In other embodiments, the label employed will generate a signal whether or not the target is specifically bound to its probe. In such embodiments, the non-bound labeled target is removed from the support surface. One means of removing the non-bound labeled target is to perform the well known technique of washing, where a variety of wash solutions and protocols for their use in removing non-bound label are known to those of skill in the art and may be used. Alternatively, non-bound labeled target can be removed by electrophoretic means.

Where all of the target sequences are detected using the same label, different arrays will be employed for each physiological source (where different could include using the same array at different times). The above methods can be varied to provide for multiplex analysis, by employing different and distinguishable labels for the different target populations (representing each of the different physiological sources being assayed). According to this multiplex method, the same array is used at the same time for each of the different target populations.

In another embodiment, hybridization is monitored in real time using a charge-coupled device (CCD) imaging camera (Guschin et al. (1997) Anal. Biochem. 250:203). Synthesis of arrays on optical fibre bundles allows easy and sensitive reading (Healy et al. (1997) Anal. Biochem. 251:270). In another embodiment, real time hybridization detection is carried out on microarrays without washing using evanescent wave effect that excites only fluorophores that are bound to the surface (see, e.g., Stimpson et al. (1995) PNAS 92:6379).

4.5.5. Hybridization of the Target Nucleic Acids to the Microarray

The next step is to contact the target nucleic acids with the array under conditions sufficient for binding between the target nucleic acids and the probes of the array. In a preferred embodiment, the target nucleic acids will be contacted with the array under conditions sufficient for hybridization to occur between the target nucleic acids and probes on the microarray, where the hybridization conditions will be selected in order to provide for the desired level of hybridization specificity.

Contact of the array and target nucleic acids involves contacting the array with an aqueous medium comprising the target nucleic acids. Contact may be achieved in a variety of different ways depending on specific configuration of the array. For example, where the array simply comprises the pattern of size separated probes on the surface of a “plate-like” rigid substrate, contact may be accomplished by simply placing the array in a container comprising the target nucleic acid solution, such as a polyethylene bag, and the like. In other embodiments where the array is entrapped in a separation media bounded by two rigid plates, the opportunity exists to deliver the target nucleic acids via electrophoretic means. Alternatively, where the array is incorporated into a biochip device having fluid entry and exit ports, the target nucleic acid solution can be introduced into the chamber in which the pattern of target molecules is presented through the entry port, where fluid introduction could be performed manually or with an automated device. In multiwell embodiments, the target nucleic acid solution will be introduced in the reaction chamber comprising the array, either manually, e.g. with a pipette, or with an automated fluid handling device.

Contact of the target nucleic acid solution and the probes will be maintained for a sufficient period of time for binding between the target and the probe to occur. Although dependent on the nature of the probe and target, contact will generally be maintained for a period of time ranging from about 10 min to 24 hrs, usually from about 30 min to 12 hrs and more usually from about 1 hr to 6 hrs.

When using commercially available microarrays, adequate hybridization conditions are provided by the manufacturer. When using non-commercial microarrays, adequate hybridization conditions can be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls.

Hybridization is carried out in conditions permitting essentially specific hybridization. The length of the probe and GC content will determine the Tm of the hybrid, and thus the hybridization conditions necessary for obtaining specific hybridization of the probe to the template nucleic acid. These factors are well known to a person of skill in the art, and can also be tested in assays. An extensive guide to the hybridization of nucleic acids is found in Tijssen (1993), “Laboratory Techniques in biochemistry and molecular biology-hybridization with nucleic acid probes.” Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)-5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5 degrees. See Tibanyenda, N. et al., Eur. J. Biochem. 139:19 (1984) and Ebel, S. et al., Biochem. 31:12083 (1992). More importantly, it is understood that as the length of the homology region increases, the effect of a single base mismatch on overall duplex stability decreases.

Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York provide a basic guide to nucleic acid hybridization.

Certain microarrays are of “active” nature, i.e., they provide independent electronic control over all aspects of the hybridization reaction (or any other affinity reaction) occurring at each specific microlocation. These devices provide a new mechanism for affecting hybridization reactions which is called electronic stringency control (ESC). Such active devices can electronically produce “different stringency conditions” at each microlocation. Thus, all hybridizations can be carried out optimally in the same bulk solution. These arrays are described in U.S. Pat. No. 6,051,380 by Sosnowski et al.

In a preferred embodiment, background signal is reduced by the use of a detergent (e.g, C-TAB) or a blocking reagent (e.g., sperm DNA, cot-1 DNA, etc.) during the hybridization to reduce non-specific binding. In a particularly preferred (embodiment, the hybridization is performed in the presence of about 0.5 mg/ml DNA (e.g., herring sperm DNA). The use of blocking agents in hybridization is well known to those of skill in the art (see, e.g., Chapter 8 in Laboratory Techniques in Biochemistry and Molecular Biology, Vol. 24: Hybridization With Nucleic Acid Probes, P. Tijssen, ed. Elsevier, N.Y., (1993)).

The method may or may not further comprise a non-bound label removal step prior to the detection step, depending on the particular label employed on the target nucleic acid. For example, in certain assay formats (e.g., “homogenous assay formats”) a detectable signal is only generated upon specific binding of target to probe. As such, in these assay formats, the hybridization pattern may be detected without a non-bound label removal step. In other embodiments, the label employed will generate a signal whether or not the target is specifically bound to its probe. In such embodiments, the non-bound labeled target is removed from the support surface. One means of removing the non-bound labeled target is to perform the well known technique of washing, where a variety of wash solutions and protocols for their use in removing non-bound label are known to those of skill in the art and may be used. Alternatively, non-bound labeled target can be removed by electrophoretic means.

Where all of the target sequences are detected using the same label, different arrays will be employed for each physiological source (where different could include using the same array at different times). The above methods can be varied to provide for multiplex analysis, by employing different and distinguishable labels for the different target populations (representing each of the different physiological sources being assayed). According to this multiplex method, the same array is used at the same time for each of the different target populations.

In another embodiment, hybridization is monitored in real time using a charge-coupled device (CCD) imaging camera (Guschin et al. (1997) Anal. Biochem. 250:203). Synthesis of arrays on optical fibre bundles allows easy and sensitive reading (Healy et al. (1997) Anal. Biochem. 251:270). In another embodiment, real time hybridization detection is carried out on microarrays without washing using evanescent wave effect that excites only fluorophores that are bound to the surface (see, e.g., Stimpson et al. (1995) PNAS 92:6379).

4.5.6. Detection of Hybridization and Analysis of Results

The above steps result in the production of hybridization patterns of target nucleic acid on the array surface. These patterns may be visualized or detected in a variety of ways, with the particular manner of detection being chosen based on the particular label of the target nucleic acid. Representative detection means include scintillation counting, autoradiography, fluorescence measurement, colorimetric measurement, light emission measurement, light scattering, and the like.

One method of detection includes an array scanner that is commercially available from Affymetrix (Santa Clara, Calif.), e.g., the 417™ Arrayer, the 418™ Array Scanner, or the Agilent GeneArray™ Scanner. This scanner is controlled from the system computer with a WindowsR interface and easy-to-use software tools. The output is a 16-bit.tif file that can be directly imported into or directly read by a variety of software applications. Preferred scanning devices are described in, e.g., U.S. Pat. Nos. 5,143,854 and 5,424,186.

When fluorescently labeled probes are used, the fluorescence emissions at each site of a transcript array can be detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, A DNA microarray system for analyzing complex DNA samples using two-color fluorescent probe hybridization, Genome Research 6:639-645). In a preferred embodiment, the arrays are scanned with a laser fluorescent scanner with a computer controlled X-Y stage and a microscope objective. Sequential excitation of the two fluorophores can be achieved with a multi-line, mixed gas laser and the emitted light is split by wavelength and detected with two photomultiplier tubes. In one embodiment in which fluorescent target nucleic acids are used, the arrays may be scanned using lasers to excite fluorescently labeled targets that have hybridized to regions of probe arrays, which can then be imaged using charged coupled devices (“CCDs”) for a wide field scanning of the array. Fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, may be used to monitor mRNA abundance levels.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, e.g., subtrackion of the background, deconvolution multi-color images, flagging or removing artifacts, verifying that controls have performed properly, normalizing the signals, interpreting fluorescence data to determine the amount of hybridized target, normalization of background and single base mismatch hybridizations, and the like. In a preferred embodiment, a system comprises a search function that allows one to search for specific patterns, e.g., patterns relating to differential gene expression, e.g., between the expression profile of a cell of R.A. and the expression profile of a counterpart normal cell in a subject. A system preferably allows one to search for patterns of gene expression between more than two samples.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively more efficient than existing ones. The accuracy of such algorithms is preferably hierarchically adjustable so that the level of detail of clustering can be systematically refined as desired.

Various algorithms are available for analyzing the gene expression profile data, e.g., the type of comparisons to perform. In certain embodiments, it is desirable to group genes that are co-regulated. This allows the comparison of large numbers of profiles. A preferred embodiment for identifying such groups of genes involves clustering algorithms (for reviews of clustering algorithms, see, e.g., Fukunaga, 1990, Statistical Pattern Recognition, 2nd Ed., Academic Press, San Diego; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, Academic Press: New York).

Clustering analysis is useful in helping to reduce complex patterns of thousands of time curves into a smaller set of representative clusters. Some systems allow the clustering and viewing of genes based on sequences. Other systems allow clustering based on other characteristics of the genes, e.g., their level of expression (see, e.g., U.S. Pat. No. 6,203,987). Other systems permit clustering of time curves (see, e.g. U.S. Pat. No. 6,263,287). Cluster analysis can be performed using the hclust routine (see, e.g., “hclust” routine from the software package S-Plus, MathSoft, Inc., Cambridge, Mass.).

In some specific embodiments, genes are grouped according to the degree of co-variation of their transcription, presumably co-regulation, as described in U.S. Pat. No. 6,203,987. Groups of genes that have co-varying transcripts are termed “genesets.” Cluster analysis or other statistical classification methods can be used to analyze the co-variation of transcription of genes in response to a variety of perturbations, e.g. caused by a disease or a drug. In one specific embodiment, clustering algorithms are applied to expression profiles to construct a “similarity tree” or “clustering tree” which relates genes by the amount of co-regulation exhibited. Genesets are defined on the branches of a clustering tree by cutting across the clustering tree at different levels in the branching hierarchy.

In some embodiments, a gene expression profile is converted to a projected gene expression profile. The projected gene expression profile is a collection of geneset expression values. The conversion is achieved, in some embodiments, by averaging the level of expression of the genes within each geneset. In some other embodiments, other linear projection processes may be used. The projection operation expresses the profile on a smaller and biologically more meaningful set of coordinates, reducing the effects of measurement errors by averaging them over each cellular constituent sets and aiding biological interpretation of the profile.

Values that can be compared include gross expression levels; averages of expression levels, e.g., from different experiments, different samples from the same subject or samples from different subjects; and ratios of expression levels, e.g., between NMD-inhibited cells and untreated control cells.

4.5.7. Data Analysis Methods

Comparison of the expression levels of one or more genes which are up-regulated in response to the inhibition of NMD with reference to expression levels in the absence of inhibition of NMD, e.g., expression levels in cells characteristic of a disease or disorder resulting from a genetic mutation or in normal counterpart cells, is preferably conducted using computer systems. In one embodiment, one or more expression levels are obtained in two cells and these two sets of expression levels are introduced into a computer system for comparison. In a preferred embodiment, one set of one or more expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one gene which is up-regulated in response to inhibition of NMD in a cell carrying a genetic mutation that causes or contributes to a disease or disorder and results in nonsense-mediated mRNA decay of the affected gene. The values can be mRNA expression levels obtained from experiments, e.g., microarray analysis. The values can also be mRNA levels normalized relative to a reference gene whose expression is constant in numerous cells under numerous conditions, e.g., GAPDH. In other embodiments, the values in the computer are ratios of, or differences between, normalized or non-normalized mRNA levels in different samples.

The computer readable medium may comprise values of at least 2, at least 3, at least 5, 10, 20, 50, 100, 200, 500 or more genes, e.g., genes listed in Tables 1-2. In a preferred embodiment, the computer readable medium comprises at least one expression profile.

Gene expression data can be in the form of a table, such as an Excel table. The data can be alone, or it can be part of a larger database, e.g., comprising other expression profiles, e.g., publicly available database. The computer readable form can be in a computer. In another embodiment, the invention provides a computer displaying the gene expression profile data.

Although the invention provides methods in which the level of expression of a single gene can be compared in two or more cells or tissue samples, in a preferred embodiment, the level of expression of a plurality of genes is compared. For example, the level of expression of at least 2, at least 3, at least 5, 10, 20, 50, 100, 200, 500 or more genes, e.g., genes listed in Tables 1-2 can be compared. In a preferred embodiment, expression profiles are compared.

In one embodiment, the invention provides a method for determining the similarity between the level of expression of one or more genes which are up-regulated in response to inhibition of NMD in a cell carrying a genetic mutation that causes or contributes to a disease or disorder and results in nonsense-mediated mRNA decay of the affected gene. The method preferably comprises obtaining the level of expression of one or more genes which are up-regulated in response to inhibition of NMD in a first cell and entering these values into a computer comprising (i) a database including records comprising values corresponding to levels of expression of one or more genes in a control untreated cell, and (ii) processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of one or more genes which are up-regulated in response to inhibition of NMD are entered into a computer system which comprises one or more databases with reference expression levels obtained from more than one cell. For example, the computer may comprise expression data of diseased and normal cells. Instructions are provided to the computer, and the computer is capable of comparing the data entered with the data in the computer to determine whether the data entered is more similar to that of a normal cell or to that of a diseased cell.

In another embodiment, the computer comprises values of expression levels in cells of subjects having a disease or disorder resulting from or contributed to by a genetic mutation at different stages of the disease or disorder and in treated (i.e. NMD-inhibited) versus untreated (control) cells, and the computer is capable of comparing expression data entered into the computer with the data stored, and produce results indicating to which of the expression data in the computer, the one entered is most similar.

In yet another embodiment, the reference expression data in the computer are expression data from cells corresponding to genes up-regulated in response to inhibition of NMD in one or more subjects having a disease or disorder, which cells are treated in vivo or in vitro with a drug used for therapy of the disease or disorder. Upon entering of expression data of a cell of a subject treated in vitro or in vivo with the drug, the computer is instructed to compare the data entered with the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

The reference expression data may also be from cells from subjects responding or not responding to several different treatments, and the computer system indicates a preferred treatment for the subject. Accordingly, the invention provides a method for selecting a therapy for a patient having a disease or disorder caused by a genetic mutation resulting in NMD, the method comprising: (i) providing the level of expression of one or more genes which are up-regulated in response to inhibition of NMD in a diseased cell of the patient; (ii) providing a plurality of reference expression levels, each associated with a therapy, wherein the subject expression levels and each reference expression level has a plurality of values, each value representing the level of expression of a gene that is up-regulated in response to inhibition of NMD; and (iii) selecting the reference expression levels most similar to the subject expression levels, to thereby select a therapy for said patient. In a preferred embodiment step (iii) is performed by a computer. The most similar reference profile may be selected by weighing a comparison value of the plurality using a weight value associated with the corresponding expression data.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes which are up—regulated in response to inhibition of NMD in a query cell with a database including records comprising reference expression of one or more reference cells and an annotation of the type of cell; and (ii) indicating to which cell the query cell is most similar based on similarities of expression levels.

The relative levels of expression, e.g., abundance of an mRNA, in two biological samples can be scored as a perturbation (relative abundance difference) or as not perturbed (i.e., the relative abundance is the same). For example, a perturbation can be a difference in expression levels between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant). Perturbations can be used by a computer for calculating and expressing comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further comprise a pointer to a descriptor of the level of expression or expression profile, e.g., from which source it was obtained, e.g., from which patient it was obtained. A descriptor can reflect the stage of disease, the therapy that the patient is undergoing or any other descriptions of the source of expression levels.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system can be an Intel Pentium®-based processor of 200 MHz or greater clock rate and with 32 MB or more of main memory. The external component may comprise a mass storage, which can be one or more hard disks (which are typically packaged together with the processor and memory). Such hard disks are typically of 1 GB or greater storage capacity. Other external components include a user interface device, which can be a monitor, together with an inputing device, which can be a “mouse”, or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system can be, for example, of the Microsoft Windows' family, such as Windows 95, Windows 98, or Windows NT. A software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes which are up-regulated in response to inhibition of NMD. The database may contain one or more expression profiles of genes which are up-regulated in response to inhibition of NMD in different cells.

In an exemplary implementation, to practice the methods of the present invention, a user first loads expression data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

4.6. GINI Diagnostic Methods

Once a specific genetic lesion is detected in one cell (e.g. from a first member of a family affected by a human genetic disease), other methods known in the art may readily be adapted for detection of this newly identified lesion in another cell population (e.g. from a second member of the family). Available methods for adaptation to GINI-based diagnostics include the polymerase chain reaction (PCR) (see U.S. Pat. Nos. 4,683,202; 4,683,195; 4,000,159; 4,965,188; 5,176,995 as well as Chehab, et al. (1987) Nature 329:293-294 and Saiki, et al. (1985) Science 230:1350-1354), the ligase chain reaction (LCR) (see Barany (1991) PNAS USA 88:189-193), the strand displacement amplification assay (SDA) (see e.g. Walker et al. (1992) Nucleic Acids Res. 20:1691) and transcription-mediated amplification (TMA) (see Jonas et al. (1993) Journal of Clinical Microbiology 31:2410-2416; and Fahy, et al. (1991) PCR Methods Appl 1: 25-33) (also known as self-sustained sequence replication (SSR)). The amplification products (amplicons) produced by PCR, LCR and SDA are DNA, whereas RNA amplicons are produced by TMA. DNA or RNA templates, generated by these protocols or others, can be analyzed for the presence of sequence variation (i.e. mutation) associated with the disease to be ascertained.

Another method, known as restriction fragment length polymorphism (RFLP), involves ascertaining whether a restriction enzyme site is present or absent at the locus of interest. In rare instances, mutations can be detected because they happen to lie within a naturally occurring restriction endonuclease recognition/cleavage site (see Bradley, et al., PCT International Publication No. WO 84/01389).

The inclusion of mismatched bases within primers used to facilitate in vitro amplification can result in the induction of artificial restriction endonuclease recognition/cleavage sites, and hence an increase in the number of loci which can be analyzed by RFLP (Cohen and Levinson (1988) Nature 334:119-124). Modified primers containing mismatched bases have been used to induce artificial recognition/cleavage sites for restriction endonucleases at critical codons within the ras gene family (see Kumar and Barbacid (1988) Oncogene 3:647-651; Todd et al. (1991) Leukemia 5:160; and Levi, et al. (1991) Cancer Res. 6:1079). The general rules for designing primers which contain mismatched bases located near the 3′ termini of primers have been established (see Kwok, et al. (1990) Nucleic Acids Research 18: 999-1005).

Any composition and device (e.g., an array) used in the above-described methods are within the scope of the invention.

In one embodiment, the invention provides a composition comprising a plurality of detection agents for detecting expression of genes which are down-regulated by NMD. In a preferred embodiment, the composition comprises at least 2, preferably at least 3, 5, 10, 20, 50, or 100 different detection agents. A detection agent can be a nucleic acid probe, e.g., DNA or RNA, or it can be a polypeptide, e.g., as antibody that binds to the polypeptide encoded by a gene characteristic of the disease or disorder. The probes can be present in equal amount or in different amounts in the solution.

A nucleic acid probe can be at least about 10 nucleotides long, preferably at least about 15, 20, 25, 30, 50, 100 nucleotides or more, and can comprise the full length gene. Preferred probes are those that hybridize specifically to genes listed in any of Tables 1-2. If the nucleic acid is short (i.e., 20 nucleotides or less), the sequence is preferably perfectly complementary to the target gene (i.e., a gene that is characteristic of the disease or disorder involving a genetic mutation that causes NMD of the gene), such that specific hybridization can be obtained. However, nucleic acids, even short ones that are not perfectly complementary to the target gene can also be included in a composition of the invention, e.g., for use as a negative control. Certain compositions may also comprise nucleic acids that are complementary to, and capable of detecting, an allele of a gene.

In a preferred embodiment, the invention provides nucleic acids which hybridize under high stringency conditions of 0.2 to 1×SSC at 65° C. followed by a wash at 0:2×SSC at 65° C. to genes which are up- or down-regulated in R.A. In another embodiment, the invention provides nucleic acids which hybridize under low stringency conditions of 6×SSC at room temperature followed by a wash at 2×SSC at room temperature. Other nucleic acids probes hybridize to their target in 3×SSC at 40 or 50° C., followed by a wash in 1 or 2×SSC at 20, 30, 40, 50, 60, or 65° C.

Nucleic acids which are at least about 80%, preferably at least about 90%, even more preferably at least about 95% and most preferably at least about 98% identical to genes which are up- or down-regulated in R.A. or cDNAs thereof, and complements thereof, are also within the scope of the invention.

Nucleic acid probes can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments. Computer programs can be used in the design of primers with the required specificity and optimal amplification properties. See, e.g., Oligo version 5.0 (National Biosciences). Factors which apply to the design and selection of primers for amplification are described, for example, by Rylchik, W. (1993) “Selection of Primers for Polymerase Chain Reaction,” in Methods in Molecular Biology, Vol. 15, White B. ed., Humana Press, Totowa, N.J.—Sequences can be obtained from GenBank or other public sources.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al., 1988, Proc. Nat. Acad. Sci. U.S.A. 85: 7448-7451), etc. In another embodiment, the oligonucleotide is a 2′-O-methylribonucleotide (Inoue et al., 1987, Nucl. Acids Res. 15: 6131-6148), or a chimeric RNA-DNA analog (Inoue et al., 1987, FEBS Lett. 215: 327-330).

“Rapid amplification of cDNA ends,” or RACE, is a PCR method that can be used for amplifying cDNAs from a number of different RNAs. The cDNAs may be ligated to an oligonucleotide linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT Pub. No. WO 97/19110.

In another embodiment, the invention provides a composition comprising a plurality of agents which can detect a polypeptide encoded by a gene characteristic of R.A. An agent can be, e.g., an antibody. Antibodies to polypeptides described herein can be obtained commercially, or they can be produced according to methods known in the art.

The probes can be attached to a solid support, such as paper, membranes, filters, chips, pins or glass slides, or any other appropriate substrate, such as those further described herein. For example, probes of genes which are up- or down-regulated in R.A. can be attached covalently or non covalently to membranes for use, e.g., in dotblots, or to solids such as to create arrays, e.g., microarrays.

4.7. GINI Therapeutic Methods

As described above, genes that are preferentially stabilized my inhibition of NMD can be used as targets in drug design and discovery. For example, assays can be conducted to identify molecules that modulate the expression and or activity of genes which are genetically mutated to cause cancer or another disease or disorder—e.g. a heritable disorder.

In one embodiment, an agent which modulates the expression of a gene of interest is identified by contacting cells expressing the gene with test compounds, and monitoring the level of expression of the gene. Alternatively, compounds which modulate the expression of gene X can be identified by conducting assays using the promoter region of a gene and screening for compounds which modify binding of proteins to the promoter region. The nucleotide sequence of the promoter may be described in a publication or available in GenBank. Alternatively, the promoter region of the gene can be isolated, e.g., by screening a genomic library with a probe corresponding to the gene. Such methods are known in the art.

Inhibitors of the polypeptide can also be agents which bind to the polypeptide, and thereby prevent it from functioning normally, or which degrades or causes the polypeptide to be degraded. For example, such an agent can be an antibody or derivative thereof which interacts specifically with the polypeptide. Preferred antibodies are monoclonal antibodies, humanized antibodies, human antibodies, and single chain antibodies. Such antibodies can be prepared and tested as known in the art.

If a polypeptide of interest binds to another polypeptide, drugs can be developed which modulate the activity of the polypeptide by modulating its binding to the other polypeptide (referred to herein as “binding partner”). Cell-free assays can be used to identify compounds which are capable of interacting with the polypeptide or binding partner, to thereby modify the activity of the polypeptide or binding partner. Such a compound can, e.g., modify the structure of the polypeptide or binding partner and thereby effect its activity. Cell-free assays can also be used to identify compounds which modulate the interaction between the polypeptide and a binding partner. In a preferred embodiment, cell-free assays for identifying such compounds consist essentially in a reaction mixture containing the polypeptide and a test compound or a library of test compounds in the presence or absence of a binding partner. A test compound can be, e.g., a derivative of a binding partner, e.g., a biologically inactive peptide, or a small molecule.

Accordingly, one exemplary screening assay of the present invention includes the steps of contacting the polypeptide or functional fragment thereof or a binding partner with a test compound or library of test compounds and detecting the formation of complexes. For detection purposes, the molecule can be labeled with a specific marker and the test compound or library of test compounds labeled with a different marker. Interaction of a test compound with a polypeptide or fragment thereof or binding partner can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the polypeptide, functional fragment thereof, polypeptide analog or binding partner is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) a polypeptide of interest, (ii) a binding partner, and (iii) a test compound; and (b) detecting interaction of the polypeptide and the binding partner. The polypeptide and binding partner can be produced recombinantly, purified from a source, e.g., plasma, or chemically synthesized, as described herein. A statistically significant change (potentiation or inhibition) in the interaction of the polypeptide and binding partner in the presence of the test compound, relative to the interaction in the absence of the test compound, indicates a potential agonist (mimetic or potentiator) or antagonist (inhibitor) of the polypeptide bioactivity for the test compound. The compounds of this assay can be contacted simultaneously. Alternatively, the polypeptide can first be contacted with a test compound for an appropriate amount of time, following which the binding partner is added to the reaction mixture. The efficacy of the compound can be assessed by generating dose response curves from data obtained using various concentrations of the test compound. Moreover, a control assay can also be performed to provide a baseline for comparison. In the control assay, isolated and purified polypeptide or binding partner is added to a composition containing the binding partner or polypeptide, and the formation of a complex is quantified in the absence of the test compound.

Complex formation between a polypeptide and a binding partner may be detected by a variety of techniques. Modulation of the formation of complexes can be quantitated using, for example, detectably labeled proteins such as radiolabeled, fluorescently labeled, or enzymatically labeled polypeptides or binding partners, by immunoassay, or by chromatographic detection.

Typically, it will be desirable to immobilize either the polypeptide or its binding partner to facilitate separation of complexes from uncomplexed forms of one or both of the proteins, as well as to accommodate automation of the assay. Binding of the polypeptide to a binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/polypeptide (GST/polypeptide) fusion proteins can be adsorbed onto glutathione sepharose beads (Sigma Chemical, St. Louis, Mo.) or glutathione derivatized microtitre plates, which are then combined with the binding partner, e.g. an ³⁵S-labeled binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of the polypeptide or binding partner found in the bead fraction quantitated from the gel using standard electrophoretic techniques such as described in the appended examples.

Other techniques for immobilizing proteins on matrices are also available for use in the subject assay. For instance, either the polypeptide or its cognate binding partner can be immobilized utilizing conjugation of biotin and streptavidin. For instance, biotinylated polypeptide molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the polypeptide can be derivatized to the wells of the plate, and the polypeptide trapped in the wells by antibody conjugation. As above, preparations of a binding partner and a test compound are incubated in the polypeptide X presenting wells of the plate, and the amount of complex trapped in the well can be quantitated. Exemplary methods for detecting such complexes, in addition to those described above for the GST-immobilized complexes, include immunodetection of complexes using antibodies reactive with the binding partner, or which are reactive with the polypeptide and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the binding partner. To illustrate, the binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes that rely on immunodetection for quantitating one of the proteins trapped in the complex, antibodies against the protein can be used. Alternatively, the protein to be detected in the complex can be “epitope tagged” in the form of a fusion protein which includes, in addition to the polypeptide sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, NJ).

In one embodiment, the effect of up-regulating the level of expression of a gene which is down-regulated in response to a genetic mutation that results in NMD of the corresponding mRNA is determined by phenotypic analysis of the cell, in particular by determining whether the cell adopts a phenotype that is more reminiscent of that of a normal cell than that of a cell characteristic of the disease or disorder associated with the genetic mutation.

In another preferred embodiment, the effect on the cell is determined by measuring the level of expression of one or more genes which are up- or down-regulated in the disease or disorder, and preferably at least about 10, or at least about 100 genes characteristic of the disease or disorder. In a preferred embodiment, the level of expression of a gene is modulated, and the level of expression of at least one gene characteristic of the disease or disorder is determined, e.g., by using a microarray having probes to the one or more genes. If the normalization of expression of the gene results in at least some normalization of the gene expression profile in the diseased cell, then normalizing the expression of the gene in a subject having the disease or disorder is expected to improve. The term “normalization of the expression of a gene in a diseased cell” refers to bringing the level of expression of that gene in the diseased cell to a level that is similar to that in the corresponding normal cell. “Normalization of the gene expression profile in a diseased cell” refers to bringing the expression profile in a diseased cell essentially to that in the corresponding non-diseased cell. In certain embodiments, the expression level of two or more genes which are up- or down-regulated in the disease or disorder is modulated and the effect on the diseased cell is determined.

A preferred cell for use in these assays is a cell characteristic of the disease or disorder that can be obtained from a subject and, e.g., established as a primary cell culture. The cell can be immortalized by methods known in the art, e.g., by expression of an oncogene or large T antigen of SV40. Alternatively, cell lines corresponding to such a diseased cell can be used. Examples include RAW cells and THP1 cells. However, prior to using such cell lines, it may be preferably to confirm that the gene expression profile of the cell line corresponds essentially to that of a cell characteristic of the disease or disorde. This can be done as described in details herein.

Modulating the expression of a gene in a cell can be achieved, e.g., by contacting the cell with an agent that increases the level of expression of the gene or the activity of the polypeptide encoded by the gene. Increasing the level of a polypeptide in a cell can also be achieved by transfecting the cell, transiently or stably, with a nucleic acid encoding the polypeptide. Decreasing the expression of a gene in a cell can be achieved by inhibiting transcription or translation of the gene or RNA, e.g., by introducing antisense nucleic acids, ribozymes or siRNAs into the cells, or by inhibiting the activity of the polypeptide encoded by the gene, e.g., by using antibodies or dominant negative mutants. These methods are further described below in the context of therapeutic methods.

A nucleic acid encoding a particular polypeptide can be obtained, e.g., by RT-PCR from a cell that is known to express the gene. Primers for the RT-PCR can be derived from the nucleotide sequence of the gene encoding the polypeptide. The nucleotide sequence of the gene is available, e.g., in GenBank or in the publications. GenBank Accession numbers of the genes listed in Tables 1-5 are provided in the tables. Amplified DNA can then be inserted into an expression vector, according to methods known in the art and transfected into diseased cells of R.A. In a control experiment, normal counterpart cells can also be transfected. The level of expression of the polypeptide in the transfected cells can be determined, e.g., by electrophoresis and staining of the gel or by Western blot using an a agent that binds the polypeptide, e.g., an antibody. The level of expression of one or more genes which are down-regulated in the disease or disorder can then be determined in the transfected cells having elevated levels of the polypeptide. In a preferred embodiment, the level of expression is determined by using a microarray. For example, RNA is extracted from the transfected cells, and used as target DNA for hybridization to a microarray, as further described herein.

4.8. Drug Design Using Microarrays

The invention also provides methods for designing and optimizing drugs for a genetic mutation, e.g., those which have been identified as described herein. In one embodiment, compounds are screened by comparing the expression level of one or more genes which are up-regulated by inhibition of NMD relative to their expression in a control untreated reference cell. In an even more preferred embodiment, the expression level of the genes is determined using microarrays, by comparing the gene expression profile of a cell treated the with a test compound with the gene expression profile of a normal counterpart cell (a “reference profile”). Optionally the expression profile is also compared to that of a cell characteristic of a disease or disorder caused by or contributed to by a genetic mutation that results nonsense-mediated mRNA decay. The comparisons are preferably done by introducing the gene expression profile data of the cell treated with the drug into a computer system comprising reference gene expression profiles which are stored in a computer readable form, using appropriate aglorithms. Test compounds will be screened for those which alter the level of expression of genes which are affected by the genetic mutation, so as to bring them to a level that is similar to that in a cell of the same type as a cell characteristic of the disease or disorder, are. Such compounds, i.e., compounds which are capable of normalizing the expression of at least about 10%, preferably at least about 20%, 50%, 70%, 80% or 90% of the genes which are affected by NMD in a cell carrying a genetic mutation that is characteristic of the disease or disorder, are candidate therapeutics.

The efficacy of the compounds can then be tested in additional in vitro assays and in vivo, in animal models. Animal models of cancer and other diseases and disorders arising from genetic mutations that cause NMD are known in the art (and see Examples). The test compound is administered to the test animal and one or more symptoms of the disease are monitored for improvement of the condition of the animal. Expression of one or more genes which are affected by NMD can also be measured before and after administration of the test compound to the animal. A normalization of the expression of one or more of these genes is indicative of the efficiency of the compound for treating the disease or disorder arising from the NMD-causing genetic muation in the animal.

The toxicity of the candidate therapeutic compound, such as resulting from a stress-related response, can be evaluated, e.g., by determining whether it induces the expression of genes known to be associated with a toxic response. Expression of such toxicity related genes may be determined in different cell types, preferably those that are known to express the genes. In a preferred method, microarrays are used for detecting changes in gene expression of genes known to be associated with a toxic response. Changes in gene expression may be a more sensitive marker of human toxicity than routine preclinical safety studies. It was shown, e.g., that a drug which was found not be to toxic in laboratory animals was toxic when administered to humans. When gene profiling was studied in cells contacted with the drug, however, it was found that a gene, whose expression is known to correlate to liver toxicity, was expressed (see below).

Such microarrays will comprise genes which are modulated in response to toxicity or stress. An exemplary array that can be used for that purpose is the Affymetrix Rat Toxicology U34 array, which contains probes of the following genes: metabolism enzymes, e.g., CYP450s, acetyltransferases, and sulfotransferases; growth factors and their receptors, e.g., IGFs, interleukins, NGTs, TGFs, and VEGT; kinases and phosphatases, e.g, lipid kinases, MAFKs, and stress-activated kinases; nuclear receptors, e.g., retinoic acid, retinoid X and PPARs; transcription factors, e.g., oncogenes, STATs, NF-kB, and zinc finger proteins; apoptosis genes, e.g., Bcl-2 genes, Bad, Bax, Caspases and Fas; stress response genes, e.g., heat-shock proteins and drug transporters; membrane proteins, e.g., gap-junction proteins and selectins; and cell-cycle regulators, e.g., cyclins and cyclin-associated proteins. Other genes included in the microarrays are only known because they contain the nucleotide sequence of an EST and because they have a connection with toxicity.

In one embodiment, a drug of interest is incubated with a cell, e.g., a cell in culture, the RNA is extracted, and expression of genes is analyzed with an array containing genes which have been shown to be up- or down-regulated in response to certain toxins. The results of the hybridization are then compared to databases containing expression levels of genes in response to certain known toxins in certain organisms. For example, the GeneLogic ToxExpress™ database can be used for that purpose. The information in this database was obtained in least in part from the use of the Affymetrix GeneChip® rat and human probe arrays with samples treated in vivo or in vitro with known toxins. The database contains levels of expression of liver genes in response to known liver toxins. These data were obtained by treating liver samples from rats treated in vivo with known toxins, and comparing the level of expression of numerous genes with that in rat or human primary hepatocytes treated in vitro with the same toxin. Data profiles can be retrieved and analyzed with the GeneExpress™ database tools, which are designed for complex data management and analysis. As indicated on the Affymetrix (Santa Clara, Calif.) website, the GeneLogic, Inc. (Gaithersburg, Md.) has preformed proof of concept studies showing the changes in gene expression levels can predict toxic events that were not identified by routine preclinical safety testing. GeneLogic tested a drug that had shown no evidence of liver toxicity in rats, but that later showed toxicity in humans. The hybridization results using the Affymetrix GeneChip® and GeneExpress tools showed that the drug caused abnormal elevations of alanine aminotransferase (ALT), which indicates liver injury, in half of the patients who had used the drug.

In one embodiment of the invention, the drug of interest is administered to an animal, such as a mouse or a rat, at different doses. As negative controls, animals are administered the vehicle alone, e.g., buffer or water. Positive controls can consist of animals treated with drugs known to be toxic. The animals can then be sacrificed at different times, e.g., at 3, 6, and 24 hours, after administration of the drug, vehicle alone or positive control drug, mRNA extracted from a sample of their liver; and the mRNA analyzed using arrays containing nucleic acids of genes which are likely to be indicative of toxicity, e.g., the Affymetrix Rat Toxicology U34 assay. The hybridization results can then be analyzed using computer programs and databases, as described above.

In addition, toxicity of a drug in a subject can be predicted based on the alleles of drug metabolizing genes that are present in a subject. Accordingly, it is known that certain enzymes, e.g., cytochrome p450 enzymes, i.e., CYP450, metabolize drugs, and thereby may render drugs which are innocuous in certain subjects, toxic in others. A commercially available array containing probes of different alleles of such drug metabolizing genes can be obtained, e.g., from Affymetrix (Santa Clara, Calif.), under the name of GeneChip® CYP450 assay.

Thus, a drug for a disease or disorder caused by a genetic mutation which results in NMD identified as described herein can be optimized by reducing any toxicity it may have. Compounds can be derivatized in vitro using known chemical methods and tested for expression of toxicity related genes. The derivatized compounds must also be retested for normalization of expression levels of genes which are down-regulated by a mutation causing NMD of the mutant mRNA. For example, the derivatized compounds can be incubated with diseased cells of an individual, and the gene expression profile determined using microarrays. Thus, incubating cells with derivatized compounds and measuring gene expression levels with a microarray that contains the genes which are affected by NMD and a microarray containing toxicity related genes, compounds which are effective in treating the disease or disorder and which are not toxic can be developed. Such compounds can further be tested in animal models as described above.

In another embodiment of the invention, a drug is developed by rational drug design, i.e., it is designed or identified based on information stored in computer readable form and analyzed by algorithms. More and more databases of expression profiles are currently being established, numerous ones being publicly available. By screening such databases for the description of drugs affecting the expression of at least some of the genes which are subject to NMD as a result of a genetic mutation associated with a disease or disorder in a manner similar to the change in gene expression profile from a cell characteristic of the disease or disorder to that of a normal counterpart cell, compounds can be identified which normalize gene expression in a cell characteristic of the genetic disease or disorder. Derivatives and analogues of such compounds can then be synthesized to optimize the activity of the compound, and tested and optimized as described above.

Compounds identified by the methods described above are within the scope of the invention. Compositions comprising such compounds, in particular, compositions comprising a pharmaceutically efficient amount of the drug in a pharmaceutically acceptable carrier are also provided. Certain compositions comprise one or more active compounds for treating the disease or disorder.

The invention also provides methods for designing therapeutics for treating diseases that arise from a genetic mutation that is different from the specific disease gene locus identified by GINI, but related thereto. Related diseases may in fact have a gene expression profile, which even though not identical to that of the specific disease gene, will show some homology, so that drugs for treating the genetic disease or disorder can be used for treating the related disease or for starting the research of compounds for treating the related disease. A compound for treating a particular genetic disease or disorder can be derivatized and tested as further described herein.

4.9. Exemplary Therapeutic Compositions

The invention provides facile therapeutic compositions based upon the gene or genes identified by GINI. Gene replacement of the missing or defective product of the thus-identified mutant gene provides therapeutic relief from the disease or disorder arising from the genetic mutation. In one embodiment, a therapeutic nucleic acid encoding a polypeptide of interest, or an equivalent thereof, such as a functionally active fragment of the polypeptide, is administered to a subject, such that the nucleic acid arrives at the site of the diseased cells, traverses the cell membrane and is expressed in the diseased cell.

A nucleic acid encoding a polypeptide of interest can be obtained as described herein, e.g., by RT-PCR, or from publicly available DNA clones. It may not be necessary to express the full length polypeptide in a cell of a subject, and a functional fragment thereof may be sufficient. Similarly, it is not necessary to express a polypeptide having an amino acid sequence that is identical to that of the wild-type polypeptide. Certain amino acid deletions, additions and substitutions are permitted, provided that the polypeptide retains most of its biological activity. For example, it is expected that polypeptides having conservative amino acid substitutions will have the same activity as the polypeptide. Polypeptides that are shorter or longer than the wild-type polypeptide or which contain from one to 20 amino acid deletions, insertions or substitutions and which have a biological activity that is essentially identical to that of the wild-type polypeptide are referred to herein as “equivalents of the polypeptide.” Equivalent polypeptides also include polypeptides having an amino acid sequence which is at least 80%, preferably at least about 90%, even more preferably at least about 95% and most preferably at least 98% identical or similar to the amino acid sequence of the wild-type polypeptide.

Determining which portion of the polypeptide is sufficient for improving the disease or disorder or which polypeptides derived from the polypeptide are “equivalents” which can be used for treating the disease or disorder, can be done in in vitro assays. For example, expression plasmids encoding various portions of the polypeptide can be transfected into cells, e.g., diseased cells of the disease or disorder., and the effect of the expression of the portion of the polypeptide in the cells can be determined, e.g., by visual inspection of the phenotype of the cell (cellular phenotype) or by obtaining the expression profile of the cell, as further described herein.

Any means for the introduction of polynucleotides into mammals, human or non-human, may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

In a preferred method of the invention, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans.

It is possible to limit the infection spectrum of viruses by modifying the viral packaging proteins on the surface of the viral particle (see, for example PCT publications WO93/25234, WO94/06920, and WO94/11524). For instance, strategies for the modification of the infection spectrum of viral vectors include: coupling antibodies specific for cell surface antigens to envelope protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral envelope proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

The expression of a polypeptide of interest or equivalent thereof in cells of a patient to which a nucleic acid encoding the polypeptide was administered can be determined, e.g., by obtaining a sample of the cells of the patient and determining the level of the polypeptide in the sample, relative to a control sample. The successful administration to a patient and expression of the polypeptide or an equivalent thereof in the cells of the patient can be monitored by determining the expression of at least one gene characteristic of a disease or disorder associated with NMD, and preferably by determining an expression profile including most of the genes which are affected by NMD, as described herein.

In another embodiment, a polypeptide of interest, or an equivalent thereof, e.g., a functional fragment thereof, is administered to the subject such that it reaches the diseased cells affected, and traverses the cellular membrane. Polypeptides can be synthesized in prokaryotes or eukaryotes or cells thereof and purified according to methods known in the art. For example, recombinant polypeptides can be synthesized in human cells, mouse cells, rat cells, insect cells, yeast cells, and plant cells. Polypeptides can also be synthesized in cell free extracts, e.g., reticulocyte lysates or wheat germ extracts. Purification of proteins can be done by various methods, e.g., chromatographic methods (see, e.g., Robert K Scopes “Protein Purification: Principles and Practice” Third Ed. Springer-Verlag, N.Y. 1994). In one embodiment, the polypeptide is produced as a fusion polypeptide comprising an epitope tag consisting of about six consecutive histidine residues. The fusion polypeptide can then be purified on a Ni⁺⁺ column. By inserting a protease site between the tag and the polypeptide, the tag can be removed after purification of the peptide on the Ni⁺⁺ column. These methods are well known in the art and commercial vectors and affinity matrices are commercially available.

Administration of polypeptides can be done by mixing them with liposomes, as described above. The surface of the liposomes can be modified by adding molecules that will target the liposome to the desired physiological location.

In one embodiment, a polypeptide is modified so that its rate of traversing the cellular membrane is increased. For example, the polypeptide can be fused to a second peptide which promotes “transcytosis,” e.g., uptake of the peptide by cells. In one embodiment, the peptide is a portion of the HIV transactivator (TAT) protein, such as the fragment corresponding to residues 37-62 or 48-60 of TAT, portions which are rapidly taken up by cell in vitro (Green and Loewenstein, (1989) Cell 55:1179-1188). In another embodiment, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides can be fused to a peptide consisting of about amino acids 42-58 of Drosophila antennapedia or shorter fragments for transcytosis. See for example Derossi et al. (1996) J Biol Chem 271:18188-18193; Derossi et al. (1994) J Biol Chem 269:10444-10450; and Perez et al. (1992) J Cell Sci 102:717-722.

In another embodiment, a pharmaceutical composition comprising a compound that stimulates the level of expression of a gene of interest or the activity of the polypeptide in a cell is administered to a subject, such that the level of expression of the gene in the diseased cells is increased or even restored.

The therapeutic compositions of the invention include the compounds described herein, e.g., in the context of therapeutic treatments of a specific disease or disorder (e.g. cancer—arising from a somatic genetic mutation). Therapeutic compositions may comprise one or more nucleic acids encoding a polypeptide characteristic of the genetic disease or disorder, or equivalents thereof. The nucleic acids may be in expression vectors, e.g., viral vectors. Other compositions comprise one or more polypeptides characteristic of the disease or disorder (i.e. a gene up-regulated in response to inhibition of NMD), or equivalents thereof. Yet other compositions comprise nucleic acids encoding antisense RNA, or ribozymes, siRNAs or RNA aptamers. Also within the scope of the invention are compositions comprising compounds identified by the methods described herein. The compositions may comprise pharmaceutically acceptable excipients, and may be contained in a device for their administration, e.g., a syringe.

4.10. Administration of Compounds and Compositions of the Invention

In a preferred embodiment, the invention provides a method for treating a subject having a disease or disorder that is associated with a genetic mutation, comprising administering to the subject a therapeutically effective amount of a pharmaceutical composition comprising a compound of the invention.

4.10.1. Effective Dose

Compounds of the invention refer to small molecules, polypeptides, peptide mimetics, nucleic acids or any other molecule identified as potentially useful for treating the genetic disease or disorder.

Toxicity and therapeutic efficacy of compounds can be determined by standard pharmaceutical procedures in cell cultures or experimental animals, e.g., for determining the LD50 (The Dose Lethal To 50% Of The Population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to healthy cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

4.10.2. Formulation

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration. In one embodiment, the compound is administered locally, at the site where the diseased cells are present, i.e., in the blood or in a joint.

The compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Administration, e.g., systemic administration, can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compounds of the invention can be formulated into ointments, salves, gels, or creams as generally known in the art. A wash solution can be used locally to treat an injury or inflammation to accelerate healing.

In clinical settings, a gene delivery system for a gene of interest can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the subject or animal being quite localized. For example, the gene delivery vehicle can be introduced by catheter (see U.S. Pat. No. 5,328,470) or by stereotactic injection (e.g., Chen et al. (1994) PNAS 91: 3054-3057). A nucleic acid, such as one encoding a polypeptide of interest or homologue thereof can be delivered in a gene therapy construct by electroporation using techniques described, for example, by Dev et al. ((1994) Cancer Treat Rev 20:105-115). Gene therapy can be conducted in vivo or ex vivo.

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

4.11. Exemplary Kits

The invention further provides kits for determining the expression level of genes characteristic of a genetic disease or disorder. The kits may be useful for identifying subjects that are predisposed to developing the genetic disease or disorder or who have the genetic disease or disorder, as well as for identifying and validating therapeutics for the genetic disease or disorder. In one embodiment, the kit comprises a computer readable medium on which is stored one or more gene expression profiles of diseased cells of the genetic disease or disorder, or at least values representing levels of expression of one or more genes which are up- or down-regulated in response to inhibition of NMD in a diseased cell. The computer readable medium can also comprise gene expression profiles of counterpart normal cells, diseased cells treated with a drug, and any other gene expression profile described herein. The kit can comprise expression profile analysis software capable of being loaded into the memory of a computer system.

The kit can also comprise one or more pharmacological or biological reagents sufficient to inhibit NMD in a test cell. Examples include: emetine, anisomycin, cycloheximide, pactamycin, puromycin, gentamicin, neomycin, paromomycin, or siRNAs (e.g. SEQ ID Nos. 1 and 2 or 3 and 4), antisense oligonucleotides or ribozymes directed against one or more components of the NMD pathway—such as RENT1 or RENT2. Other agents for inhibition of NMD in a test cell which may be included in the kit include dominant negative components of the NMD pathway such as a dominant negative RENT1 which carries an arg to cys mutation at the RENT1 amino acid residue 843 (e.g. SEQ ID No. 6).

A kit can comprise a microarray comprising probes of genes which are up- or down-regulated in response to inhibition of NMD. A kit can comprise one or more probes or primers for detecting the expression level of one or more genes which are up- or down-regulated in response to inhibition of NMD and/or a solid support on which probes attached and which can be used for detecting expression of one or more genes which are up- or down-regulated in response to inhibition of NMD in a sample. A kit may further comprise nucleic acid controls, buffers, and instructions for use.

Other kits provide compositions for treating the disease or disorder resulting from the genetic mutation that causes NMD. For example, a kit can also comprise one or more nucleic acids corresponding to one or more genes which are up- or down-regulated in response to inhibition of NMD, e.g., for use in treating a patient having the disease or disorder. The nucleic acids can be included in a plasmid or a vector, e.g., a viral vector. Other kits comprise a polypeptide encoded by a gene characteristic of a disease or disorder or an antibody to a polypeptide. Yet other kits comprise compounds identified herein as agonists or antagonists of genes which are up- or down-regulated in the disease or disorder. The compositions may be pharmaceutical compositions comprising a pharmaceutically acceptable excipient.

4.12. Nucleic Acids

The invention provides NMD-inhibitory activity-encoding and other nucleic acids, homologs thereof, and portions thereof. Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and more preferably 95% and even more preferably at least 99% homologous with a nucleotide sequence of a subject gene, e.g., an NMD pathway-encoding gene Nucleic acids at least 90%, more preferably 95%, and most preferably at least about 98-99% identical with a nucleic sequence represented in one of the subject nucleic acids of the invention or complement thereof are of course also within the scope of the invention. In preferred embodiments, the nucleic acid is mammalian and in particularly preferred embodiments, includes all or a portion of the nucleotide sequence corresponding to the coding region which correspond to the coding sequences of the subject NMD pathway-encoding DNAs.

The invention also pertains to isolated nucleic acids comprising a nucleotide sequence encoding NMD pathway polypeptides, variants and/or equivalents of such nucleic acids. The term equivalent is understood to include nucleotide sequences encoding functionally equivalent NMD pathway polypeptides or functionally equivalent peptides having an activity of an NMD pathway protein such as described herein. Equivalent nucleotide sequences will include sequences that differ by one or more nucleotide substitution, addition or deletion, such as allelic variants; and will, therefore, include sequences that differ from the nucleotide sequences of e.g. the corresponding NMD pathway gene GenBank entries due to the degeneracy of the genetic code.

Preferred nucleic acids are vertebrate NMD pathway nucleic acids. Particularly preferred vertebrate NMD pathway nucleic acids are mammalian. Regardless of species, particularly preferred NMD pathway nucleic acids encode polypeptides that are at least 60%, 65%, 70%, 72%, 74%, 76%, 78%, 80%, 90%, or 95% similar or identical to an amino acid sequence of a vertebrate NMD pathway protein. In one embodiment, the nucleic acid is a cDNA encoding a polypeptide having at least one bio-activity of the subject NMD pathway polypeptides or APC-stimulatory factors. Preferably, the nucleic acid includes all or a portion of the nucleotide sequence corresponding to the nucleic acids available through GenBank.

Still other preferred nucleic acids of the present invention encode an NMD pathway-encoding polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length.

Another aspect of the invention provides a nucleic acid which hybridizes under stringent conditions to a nucleic acid represented by any of the subject nucleic acids of the invention. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. In a preferred embodiment, an NMD pathway nucleic acid of the present invention will bind to one of the subject SEQ ID Nos. or complement thereof under moderately stringent conditions, for example at about 2.0×SSC and about 40° C. In a particularly preferred embodiment, an NMD pathway-encoding nucleic acid of the present invention will bind to one of the nucleic acid sequences of SEQ ID Nos. 5, 7 or 8 or complement thereof under high stringency conditions. In another particularly preferred embodiment, an NMD pathway-encoding nucleic acid sequence of the present invention will bind to one of the nucleic acids of the invention which correspond to an NMD pathway-encoding ORF nucleic acid sequences, under high stringency conditions.

Nucleic acids having a sequence that differs from the nucleotide sequences shown in one of the nucleic acids of the invention or complement thereof due to degeneracy in the genetic code are also within the scope of the invention. Such nucleic acids encode functionally equivalent peptides (i.e., peptides having a biological activity of an NMD pathway-encoding polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of an NMD pathway polypeptide. However, it is expected that DNA sequence polymorphisms that do lead to changes in the amino acid sequences of the subject NMD pathway polypeptides will exist among mammals. One skilled in the art will appreciate that these variations in one or more nucleotides (e.g., up to about 3-5% of the nucleotides) of the nucleic acids encoding polypeptides having an activity of an NMD pathway-encoding polypeptide may exist among individuals of a given species due to natural allelic variation.

4.12.1 Probes and Primers

The nucleotide sequences determined from the cloning of NMD pathway genes from mammalian organisms will further allow for the generation of probes and primers designed for use in identifying and/or cloning other NMD pathway homologs in other cell types, e.g., from other tissues, as well as NMD pathway homologs from other mammalian organisms. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence selected from one of the nucleic acids (e.g. an NMD pathway-encoding nucleic acid) of the invention.

In preferred embodiments, the NMD pathway primers are designed so as to optimize specificity and avoid secondary structures which affect the efficiency of priming. Optimized PCR primers of the present invention are designed so that “upstream” and “downstream” primers have approximately equal melting temperatures such as can be estimated using the formulae: Tm=81.5 C−16.6(log 10[Na+])+0.41(% G+C)−0.63 (% formamide)−(600/length); or Tm(C)=2(A/T)+4(G/C). Optimized NMD pathway primers may also be designed by using various programs, such as “Primer3” provided by the Whitehead Institute for Bi

Likewise, probes based on the subject NMD pathway sequences can be used to detect transcripts or genomic sequences encoding the same or homologous proteins, for use, e.g, in prognostic or diagnostic assays (further described below). The invention provides probes which are common to alternatively spliced variants of the NMD pathway transcript, such as those corresponding to at least 12 consecutive nucleotides complementary to a sequence found in any of the gene sequences of the invention. In addition, the invention provides probes which hybridize specifically to alternatively spliced forms of the NMD pathway transcript. Probes and primers can be prepared and modified, e.g., as previously described herein for other types of nucleic acids.

4.13. Polypeptides

The present invention makes available isolated NMD pathway polypeptides which are isolated from, or otherwise substantially free of other cellular proteins. The term “substantially free of other cellular proteins” (also referred to herein as “contaminating proteins”) or “substantially pure or purified preparations” are defined as encompassing preparations of NMD pathway polypeptides having less than about 20% (by dry weight) contaminating protein, and preferably having less than about 5% contaminating protein. Functional forms of the subject polypeptides can be prepared, for the first time, as purified preparations by using a cloned gene as described herein.

Preferred NMD pathway proteins of the invention have an amino acid sequence which is at least about 60%, 65%, 66%, 67%, 68%, 69%, 70%, 71%, 72%, 73%, 74%, 75%, 76%, 77%, 78%, 79%, 80%, 85%, 90%, or 95% identical or homologous to an amino acid sequence of a SEQ ID No. of the invention, such as a sequence shown in SEQ ID Nos. 5, 7 or 8. Even more preferred NMD pathway proteins comprise an amino acid sequence of at least 10, 20, 30, or 50 residues which is at least about 70, 80, 90, 95, 97, 98, or 99% homologous or identical to an amino acid sequence of a protein encoded by SEQ ID Nos. 5, 7 or 8 of the invention. Such proteins can be recombinant proteins, and can be, e.g., produced in vitro from nucleic acids comprising a nucleotide sequence set forth in SEQ ID Nos. 5, 7 or 8 of the invention or homologs thereof. For example, recombinant polypeptides preferred by the present invention can be encoded by a nucleic acid, which is at least 85% homologous and more preferably 90% homologous and most preferably 95% homologous with a nucleotide sequence set forth in a SEQ ID Nos. 5, 7 or 8 of the invention. Polypeptides which are encoded by a nucleic acid that is at least about 98-99% homologous with the sequence of a SEQ ID Nos. 5, 7 or 8 of the invention are also within the scope of the invention.

In a preferred embodiment, an NMD pathway protein of the present invention is a mammalian NMD pathway protein. In a particularly preferred embodiment an NMD pathway protein is set forth as a SEQ ID No. of the invention. In particularly preferred embodiments, an NMD pathway protein has an NMD pathway bioactivity. It will be understood that certain post-translational modifications, e.g., phosphorylation and the like, can increase the apparent molecular weight of the NMD pathway protein relative to the unmodified polypeptide chain.

The invention also features protein isoforms encoded by splice variants of the present invention. Such isoforms may have biological activities identical to or different from those possessed by the NMD pathway proteins specified by, e.g. SEQ ID No. 6, or encoded by a nucleic acid encoded by a SEQ ID No. of the invention. Such isoforms may arise, for example, by alternative splicing of one or more NMD pathway gene transcripts.

NMD pathway polypeptides preferably are capable of functioning as either an agonist or antagonist of at least one biological activity of a wild-type (“authentic”) NMD pathway protein of the appended sequence listing. The term “evolutionarily related to”, with respect to amino acid sequences of NMD pathway proteins, refers to both polypeptides having amino acid sequences which have arisen naturally, and also to mutational variants of human NMD pathway polypeptides which are derived, for example, by combinatorial mutagenesis.

Full length proteins or fragments corresponding to one or more particular motifs and/or domains or to arbitrary sizes, for example, at least 5, 10, 20, 25, 50, 75 and 100, amino acids in length are within the scope of the present invention.

For example, isolated NMD pathway polypeptides can be encoded by all or a portion of a nucleic acid sequence shown in any of SEQ ID Nos. 5, 7 or 8 of the invention. Isolated peptidyl portions of NMD pathway proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, an NMD pathway polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) NMD pathway protein.

An NMD pathway polypeptide can be a membrane bound form or a soluble form. A preferred soluble NMD pathway polypeptide is a polypeptide which does not contain a hydrophobic signal sequence domain. Such proteins can be created by genetic engineering by methods known in the art. The solubility of a recombinant polypeptide may be increased by deletion of hydrophobic domains, such as predicted transmembrane domains, of the wild type protein.

In general, polypeptides referred to herein as having an activity (e.g., are “bioactive”) of an NMD pathway protein are defined as polypeptides which include an amino acid sequence encoded by all or a portion of the nucleic acid sequences shown in one of the subject SEQ ID Nos. and which mimic or antagonize all or a portion of the biological/biochemical activities of a naturally occurring NMD pathway protein. Examples of such biological activity include a region of conserved structure.

Other biological activities of the subject NMD pathway proteins will be reasonably apparent to those skilled in the art. According to the present invention, a polypeptide has biological activity if it is a specific agonist or antagonist of a naturally-occurring form of an NMD pathway protein.

Assays for determining whether a compound, e.g, a protein, such as an NMD pathway protein or variant thereof, has one or more of the above biological activities include those assays, well known in the art, which are used for assessing NMD pathway agonist and NMD pathway antagonist activities.

Other preferred proteins of the invention are those encoded by the nucleic acids set forth in the section pertaining to nucleic acids of the invention. In particular, the invention provides fusion proteins, e.g., NMD pathway-immunoglobulin fusion proteins. Such fusion proteins can provide, e.g., enhanced stability and solubility of NMD pathway proteins and may thus be useful in therapy. Fusion proteins can also be used to produce an immunogenic fragment of an NMD pathway protein. For example, the VP6 capsid protein of rotavirus can be used as an immunologic carrier protein for portions of the NMD pathway polypeptide, either in the monomeric form or in the form of a viral particle. The nucleic acid sequences corresponding to the portion of a subject NMD pathway protein to which antibodies are to be raised can be incorporated into a fusion gene construct which includes coding sequences for a late vaccinia virus structural protein to produce a set of recombinant viruses expressing fusion proteins comprising NMD pathway epitopes as part of the virion. It has been demonstrated with the use of immunogenic fusion proteins utilizing the Hepatitis B surface NMD pathway fusion proteins that recombinant Hepatitis B virions can be utilized in this role as well. Similarly, chimeric constructs coding for fusion proteins containing a portion of an NMD pathway protein and the poliovirus capsid protein can be created to enhance immunogenicity of the set of polypeptide NMD pathways (see, for example, EP Publication No: 0259149; and Evans et al. (1989) Nature 339:385; Huang et al. (1988) J. Virol. 62:3855; and Schlienger et al. (1992), J. Virol. 66:2).

The Multiple NMD pathway peptide system for peptide-based immunization can also be utilized to generate an immunogen, wherein a desired portion of an NMD pathway polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). NMD pathway ic determinants of NMD pathway proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the NMD pathway polypeptides of the present invention. For example, NMD pathway polypeptides can be generated as glutathione-S-transferase (GST-fusion) proteins. Such GST-fusion proteins can enable easy purification of the NMD pathway polypeptide, as for example by the use of glutathione-derivatized matrices (see, for example, Current Protocols in Molecular Biology, eds. Ausubel et al. (N.Y.: John Wiley & Sons, 1991)). Additionally, fusion of NMD pathway polypeptides to small epitope tags, such as the FLAG or hemagluttinin tag sequences, can be used to simplify immunological purification of the resulting recombinant polypeptide or to facilitate immunological detection in a cell or tissue sample. Fusion to the green fluorescent protein, and recombinant versions thereof which are known in the art and available commercially, may further be used to localize NMD pathway polypeptides within living cells and tissue.

The present invention further pertains to methods of producing the subject NMD pathway polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant NMD pathway polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant NMD pathway polypeptide is a fusion protein containing a domain which facilitates its purification, such as GST fusion protein.

Moreover, it will be generally appreciated that, under certain circumstances, it may be advantageous to provide homologs of one of the subject NMD pathway polypeptides which function in a limited capacity as one of either an NMD pathway agonist (mimetic) or an NMD pathway antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of NMD pathway proteins.

Homologs of each of the subject NMD pathway proteins can be generated by mutagenesis, such as by discrete point mutation(s), or by truncation. For instance, mutation can give rise to homologs which retain substantially the same, or merely a subset, of the biological activity of the NMD pathway polypeptide from which it was derived. Alternatively, antagonistic forms of the protein can be generated which are able to inhibit the function of the naturally occurring form of the protein, such as by competitively binding to an NMD pathway receptor.

The recombinant NMD pathway polypeptides of the present invention also include homologs of the wildtype NMD pathway proteins, such as versions of those protein which are resistant to proteolytic cleavage, as for example, due to mutations which alter ubiquitination or other enzymatic targeting associated with the protein.

NMD pathway polypeptides may also be chemically modified to create NMD pathway derivatives by forming covalent or aggregate conjugates with other chemical moieties, such as glycosyl groups, lipids, phosphate, acetyl groups and the like. Covalent derivatives of NMD pathway proteins can be prepared by linking the chemical moieties to functional groups on amino acid sidechains of the protein or at the N-terminus or at the C-terminus of the polypeptide.

Modification of the structure of the subject NMD pathway polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the NMD pathway polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, WH Freeman and Co.: 1981). Whether a change in the amino acid sequence of a peptide results in a functional NMD pathway homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

This invention further contemplates a method for generating sets of combinatorial mutants of the subject NMD pathway proteins as well as truncation mutants, and is especially useful for identifying potential variant sequences (e.g., homologs). The purpose of screening such combinatorial libraries is to generate, for example, novel NMD pathway homologs which can act as either agonists or antagonist, or alternatively, possess novel activities all together. Thus, combinatorially-derived homologs can be generated to have an increased potency relative to a naturally occurring form of the protein.

In one embodiment, the variegated NMD pathway libary of NMD pathway variants is generated by combinatorial mutagenesis at the nucleic acid level, and is encoded by a variegated gene NMD pathway library. For instance, a mixture of synthetic oligonucleotides can be enzymatically ligated into gene sequences such that the degenerate set of potential NMD pathway sequences are expressible as individual polypeptides, or alternatively, as a set of larger fusion proteins (e.g., for phage display) containing the set of NMD pathway sequences therein.

There are many ways by which such libraries of potential NMD pathway homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential NMD pathway sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for an NMD pathway clone in order to generate a variegated population of NMD pathway fragments for screening and subsequent selection of bioactive fragments. A variety of techniques are known in the art for generating such 1, including chemical synthesis. In one embodiment, a library of coding sequence fragments can be generated by (i) treating a double stranded PCR fragment of an NMD pathway coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of NMD pathway homologs. The most widely used techniques for screening large gene libraries typically comprises cloning the gene library into replicable expression vectors, transforming appropriate cells with the resulting libraries of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate NMD pathway sequences created by combinatorial mutagenesis techniques. Combinatorial mutagenesis has a potential to generate very large libraries of mutant proteins, e.g., in the order of 1026 molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the NMD pathway proteins to generate mimetics, e.g., peptide or non-peptide agents, such as small molecules, which are able to disrupt binding of an NMD pathway polypeptide of the present invention with a molecule, e.g. target peptide. Thus, such mutagenic techniques as described above are also useful to map the determinants of the NMD pathway proteins which participate in protein-protein interactions involved in, for example, binding of the subject NMD pathway polypeptide to a target peptide. To illustrate, the critical residues of a subject NMD pathway polypeptide which are involved in molecular recognition of its receptor can be determined and used to generate NMD pathway derived peptidomimetics or small molecules which competitively inhibit binding of the authentic NMD pathway protein with that moiety. By employing, for example, scanning mutagenesis to map the amino acid residues of the subject NMD pathway proteins which are involved in binding other proteins, peptidomimetic compounds can be generated which mimic those residues of the NMD pathway protein which facilitate the interaction. Such mimetics may then be used to interfere with the normal function of an NMD pathway protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun 126:419; and Dann et al. (1986) Biochem Biophys Res Commun 134:71).

“Encoded by” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences which are immunologically identifiable with a polypeptide encoded by the sequence. Thus, an NMD pathway “polypeptide,” “protein,” or “amino acid” sequence may have at least 60% similarity, preferably at least about 75% similarity, more preferably about 85% similarity, and most preferably about 95% similarity, to a polypeptide or amino acid sequence of an NMD pathway. This amino acid sequence can be selected from the group consisting of the polypeptide sequence encoded by SEQ ID Nos. 5, 7 or 8.

A “recombinant polypeptide” or “recombinant protein” or “polypeptide produced by recombinant techniques,” which are used interchangeably herein, describes a polypeptide which by virtue of its origin or manipulation is not associated with all or a portion of the polypeptide with which it is associated in nature and/or is linked to a polypeptide other than that to which it is linked in nature. A recombinant or encoded polypeptide or protein is not necessarily translated from a designated nucleic acid sequence. It also may be generated in any manner, including chemical synthesis or expression of a recombinant expression system.

The term “synthetic peptide” as used herein means a polymeric form of amino acids of any length, which may be chemically synthesized by methods well-known to the routineer. These synthetic peptides are useful in various applications.

The term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as, double- and single-stranded RNA. It also includes modifications, such as methylation or capping, and unmodified forms of the polynucleotide. The terms “polynucleotide,” “oligomer,” “oligonucleotide,” and “oligo” are used interchangeably herein.

“A sequence corresponding to a cDNA” means that the sequence contains a polynucleotide sequence that is identical to or complementary to a sequence in the designated DNA. The degree (or “percent”) of identity or complementarity to the cDNA will be approximately 50% or greater, will preferably be at least about 70% or greater, and more preferably will be at least about 90%. The sequence that corresponds to the identified cDNA will be at least about 50 nucleotides in length, will preferably be about 60 nucleotides in length, and more preferably, will be at least about 70 nucleotides in length. The correspondence between the gene or gene fragment of interest and the cDNA can be determined by methods known in the art, and include, for example, a direct comparison of the sequenced material with the cDNAs described, or hybridization and digestion with single strand nucleases, followed by size determination of the digested fragments.

“Purified polynucleotide” refers to a polynucleotide of interest or fragment thereof which is essentially free, i.e., contains less than about 50%, preferably less than about 70%, and more preferably, less than about 90% of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

“Purified polypeptide” means a polypeptide of interest or fragment thereof which is essentially free, that is, contains less than about 50%, preferably less than about 70%, and more preferably, less than about 90% of cellular components with which the polypeptide of interest is naturally associated. Methods for purifying are known in the art.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Polypeptide” and “protein” are used interchangeably herein and indicates a molecular chain of amino acids linked through covalent and/or noncovalent bonds. The terms do not refer to a specific length of the product. Thus, peptides, oligopeptides and proteins are included within the definition of polypeptide. The terms include post-expression modifications of the polypeptide, for example, glycosylations, acetylations, phosphorylations and the like. In addition, protein fragments, analogs, mutated or variant proteins, fusion proteins and the like are included within the meaning of polypeptide.

A “fragment” of a specified polypeptide refers to an amino acid sequence which comprises at least about 3-5 amino acids, more preferably at least about 8-10 amino acids, and even more preferably at least about 15-20 amino acids, derived from the specified polypeptide.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

As used herein “replicon” means any genetic element, such as a plasmid, a chromosome or a virus, that behaves as an autonomous unit of polynucleotide replication within a cell.

A “vector” is a replicon in which another polynucleotide segment is attached, such as to bring about the replication and/or expression of the attached segment.

The term “control sequence” refers to polynucleotide sequences which are necessary to effect the expression of coding sequences to which they are ligated. The nature of such control sequences differs depending upon the host organism. In prokaryotes, such control sequences generally include promoter, ribosomal binding site and terminators; in eukaryotes, such control sequences generally include promoters, terminators and, in some instances, enhancers. The term “control sequence” thus is intended to include at a minimum all components whose presence is necessary for expression, and also may include additional components whose presence is advantageous, for example, leader sequences.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequences.

The term “open reading frame” or “ORF” refers to a region of a polynucleotide sequence which encodes a polypeptide; this region may represent a portion of a coding sequence or a total coding sequence.

A “coding sequence” is a polynucleotide sequence which is transcribed into mRNA and translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences.

4.14. Further Practice of the Invention

The present invention is further illustrated by the following examples which should not be construed as limiting in any way. The contents of all cited references including literature references, issued patents, published and non published patent applications as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### 5. EXAMPLES

Use of GINI to Identify Nonsense Mutation-Carrying Disease Genes

Premature termination codons (PTCS) have been shown to initiate degradation of mutant transcripts through the nonsense-mediated messenger RNA (mRNA) decay (NMD) pathway. In this example, we demonstrate a method, termed gene identification by NMD inhibition (GINI), to identify genes harboring nonsense codons that underlie human diseases. In this strategy, the NMD pathway is pharmacologically inhibited in cultured patient cells, resulting in stabilization of nonsense transcripts. To distinguish stabilized nonsense transcripts from background transcripts upregulated by drug treatment, drug-induced expression changes are measured in control and disease cell lines with complementary DNA (cDNA) microarrays. Transcripts are ranked by a nonsense enrichment index (NEI), which relates expression changes for a given transcript in NMD-inhibited control and patient cell lines. The most promising candidates can be selected using information such as map location or biological function; however, an important advantage of the GINI strategy is that a priori information is not essential for disease gene identification. GINI was tested on colon cancer and Sandhoff disease cell lines, which contained previously characterized nonsense mutations in the MutL homolog 1 (MLH1) and hexosaminidase B (HEXB) genes, respectively. A list of genes was produced in which the MLH1 and HEXB genes were among the top 1% of candidates, thus validating the strategy.

An estimated one-third of mutations underlying human disorders result in premature termination codons, which subsequently lead to rapid degradation of the mutant mRNA by the NMD pathway (see Losson et al. (1979) PNAS USA 76: 5134-37; Culbertson et al. (1999) Trends Genet 15: 74-80; and Frischmeyer and Dietz (1999) Hum Mol Genet 8: 1893-1900). Although the molecular mechanisms of NMD are not fully understood, the pathway utilizes trans-factors that associate with polysomes (Atkin et al. (1995) Mol Biol Cell 6: 611-25; Atkin et al. (1997) J Biol Chem 272: 22163-72) and is inhibited by experimental manipulations that impair the efficiency of translation (Qian et al. (1993) Mol Cell Biol 13: 1686-96; Carter et al. (1995) J Biol Chem 270: 28995-29003).

A conventional strategy for identification of disease genes is to use microarrays to compare the level of gene-specific mRNA expression between patient and control samples. Inter-individual variation and secondary changes in gene expression caused by the disease process can obscure identification of the mutated gene. Here, we demonstrate an alternative strategy that circumvents these limitations, called GINI. The patient sample is compared to itself after pharmacological inhibition of NMD. Microarrays are then used to identify potential nonsense transcripts that are increased in abundance after loss of NMD.

The GINI strategy was tested on two cell lines containing previously characterized nonsense mutations. The gene-specific drug-induced fold changes in the patient lines were divided by the fold changes in control fibroblast lines, producing a score termed the NEI, by which the transcripts were ordered. Both nonsense transcripts ranked in the top 1% of candidates. This work represents a proof of concept that the GINI strategy can be used to identify genes that underlie human disease.

Experimental Protocols

Cell Culture

Primary fibroblasts were grown in minimal essential medium (MEM; Life Technologies, Gaithersburg, Md.), 15% fetal bovine serum (FBS; Biofluids, Rockville, Md.), 0.1 U/ml antibiotic-antimycotic (Life Technologies), 2 mM glutamine (Life Technologies); colon cancer cells were grown in McCoys 5A Media (Life Technologies), 10% FBS (Biofluids), 0.1 U/ml antibiotic-antimycotic (Life Technologies); and prostate cancer cells were grown in RPMI 1640 medium (Life Technologies), 15% FBS (Biofluids), 0.1 U/ml antibiotic-antimycotic (Life Technologies).

Cell Lines

Cell lines were obtained from the following sources: 203 fibroblasts, compound heterozygote for frameshifts in HEXB: delta G774 in exon 7, and delta AG1305-1306 in exon 11 (Repository number GM00203A, NIGMS Human Genetic Mutant Cell Repository, Coriell Institute for Medical Research, Camden, N.J.); CON1-5, male primary skin fibroblasts; HCT116, homozygous nonsense mutation S252X in MLH1, and DLD1, compound heterozygote frameshift in GTBP: 1 bp deletion at codon 222 and 5 bp deletion at codon 1103 (gifts from Dr. Ken Kinzler and Dr. Bert Vogelstein, Johns Hopkins Oncology Center, Baltimore, Md.); PC3, monoallelic frameshift delta C codon 138 in TP53, and LnCAP (American Type Culture Collection (ATCC), Rockville, Md.).

Drugs

Drugs were obtained from the following sources: anisomycin, cycloheximide, emetine, paromomycin, puromycin (Sigma, St. Louis, Mo.); gentamicin (Quality Biological, Inc., Gaithersburg, Md.); neomycin (Life Technologies); pactamycin (gift from the Drug Synthesis and Chemistry Branch, National Cancer Institute, Bethesda, Md.).

RNA Isolation and Northern Blots

Total RNA was used in drug titration and time course experiments and was isolated with TRIZOL (Life Technologies). Poly (A)+ mRNA was used to identify NEI scores and was isolated by double purification of total RNA with the Oligotex mRNA Kit (Qiagen, Valencia, Calif.). For northern blots, 0.5 g of mRNA was separated on 1.2% agarose formaldehyde gels, and transferred to a nylon membrane (GeneScreen Plus, NEN, Boston, Mass.). Hybridizations with radiolabeled probes were carried out at 68° C. using ExpressHyb Hybridization Solution (Clontech, Palo Alto, Calif.). Signal intensities were measured with an Instant Imager (Packard Instrument Company, Meriden, Conn.).

The G3PDH probe was purchased from Clontech (catalog # 9805-1). All other probes were generated by PCR of either plasmid or mRNA-derived cDNA. Primer sequences are available upon request. The HEXB probe was derived from clone PHEXB43 (ATCC). The TP53, MLH1, and GTBP probes were derived from plasmids provided by Ken Kinzler and Bert Vogelstein (Johns Hopkins Oncology Center), and the 7S rRNA probe was synthesized (5′-GAGACGGGGTCTCGCTATGTTGCC-3′).

Microarray Analysis

For the Incyte microarrays, cRNA labeling and hybridization to Unigem V1.0 microarrays was performed as a routine commercial service (catalog #Gem-5100) by Incyte Genomics (Palo Alto, Calif.). For Affymetrix (Santa Clara, Calif.) microarrays, hybridization services (protocol AFFY.HuFL) were provided by Research Genetics (Huntsville, Ky). All expression data were analyzed using the Microsoft Excel program, and cytogenetic locations were identified using DRAGON (Database Referencing of Array Genes ONline)23.

RNAi

To inhibit RENT1 expression, we used siRNAs composed of the following complementary RNA strands:

| | | |
|---|---|---|
| sense strand - | 5′ GAUGCAGUUCCGCUCCAUUdTdT 3′ | (SEQ ID NO. 1) |
| and | | |
| antisense strand - | 5′ AAUGGAGCGGAACUGCAUCdTdT 3′, | (SEQ ID NO. 2) |
| which form the 19 bp ds siRNA: | GAUGCAGUUCCGCUCCAUUdTdT | |
| | dTdTCUACGUCAAGGCGAGGUAA | |

To inhibit RENT2 expression, we used siRNAs composed of the following complementary RNA strands:

| | | |
|---|---|---|
| sense strand - | 5′ GGCUUUUGUCCCAGCCAUCdTdT 3′ | (SEQ ID NO. 3) |
| and | | |
| antisense strand - | 5′ GAUGGCUGGGACAAAAGCCdTdT 3′, | (SEQ ID NO. 4) |
| which form the 19 bp ds siRNA: | GGCUUUUGUCCCAGCCAUCdTdT | |
| | dTdTCCGAAAACAGGGUCGGUAG | |

Results

Pharmacological Stabilization of Nonsense Transcripts

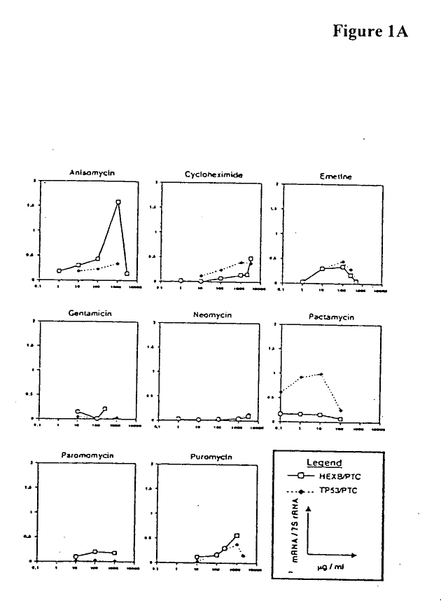

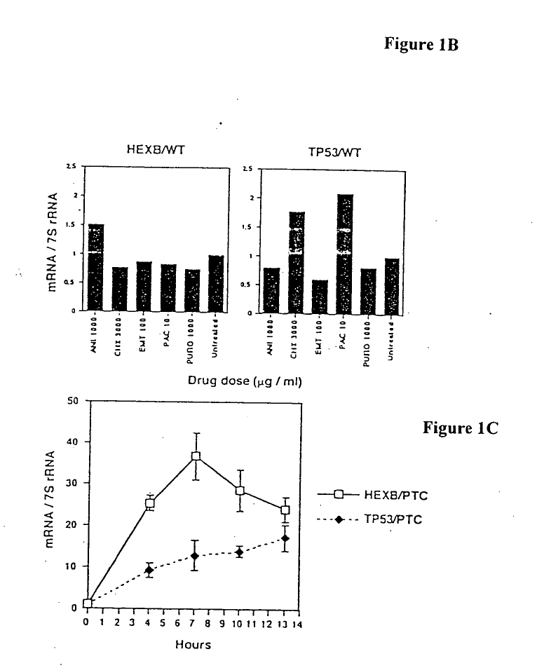

An ideal agent for the GINI strategy would be consistently effective in inhibition of NMD and minimally consequential to wild-type transcripts. Eight drugs were tested on two cell lines carrying known nonsense transcripts. 203 fibroblasts and PC3 prostate cancer cells are compound heterozygous for nonsense alleles in the HEXB gene and monoallelic for a nonsense allele in the tumor protein P53 (TP53) gene, respectively. The drugs included the translation inhibitors anisomycin, cycloheximide, emetine, pactamycin, and puromycin and the aminoglycosides gentamicin, neomycin, and paromomycin, which have been shown to cause translational readthrough of nonsense mutations (Martin et al. (1989) Mol Gen Genet 217: 411-18). Cultured cells were incubated for 10 h in the presence of multiple doses of each drug. Northern blot analysis was used to determine the relative steady-state abundance of transcripts in untreated and treated cells (FIG. 1A). The translation-inhibiting drugs had a greater stabilizing effect than the aminoglycosides, which did not cause appreciable transcript stabilization. Most of the stabilizing effects of the translation inhibitors were similar; however, anisomycin and pactamycin had discordant effects on the two test transcripts. Anisomycin greatly stabilized the nonsense HEXB transcript (HEXB/PTC) but not the nonsense TP53 transcript (TP53/PTC). Conversely, pactamycin had a strong stabilizing effect on TP53/PTC but not on HEXB/PTC.

To determine the basis for this discrepancy, the most effective stabilizing doses of the five translation inhibitors were tested using the corresponding wild-type transcripts from a primary fibroblast cell line, CON2 (FIG. 1B). The results show that 1,000 ug/ml anisomycin increased HEXB/WT whereas 10 ug/ml pactamycin and 3,000 ug/ml cycloheximide increased TP53/WT levels. All other drugs had minimal or inhibitory effects on the levels of the wild-type transcripts. The upregulatory effects of anisomycin and pactamycin on the wild-type transcripts may result from an increase in mRNA stability or transcription and likely explains their disproportionate upregulatory effects on HEXB/PTC and TP53/PTC. Emetine and puromycin remained as attractive agents for GINI because of their robust and selective effects on both test nonsense transcripts. We selected emetine at a dose of 100 ug/ml. We chose 10 h as the experimental treatment time because this interval permitted substantial accumulation of nonsense transcripts and because significant cell death had routinely occurred by the 10 h time point (FIG. 1C).

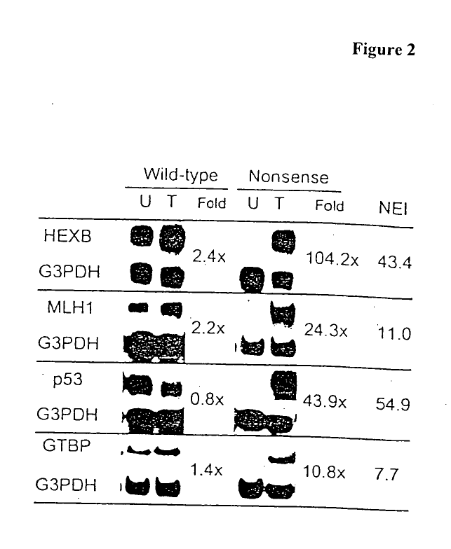

To further evaluate emetine's effects on nonsense transcripts, we incubated the cell lines 203, HCT116, DLD1, and PC3, containing nonsense mutations in the HEXB, MLH1, G/T mismatch binding protein (GTBP) (see Ohzeki et al. (1997) Carcinogenesis 18: 1127-33), and TP53 (Isaacs et al. (1991) 18: 1127-33) genes, respectively, with 100 ug/ml emetine for 10 h (FIG. 2). Drug-induced changes were determined for the nonsense transcripts and their wild-type counterparts. Stabilization of the nonsense transcripts ranged from approximately 10 to 100 fold when standardized to the glucose 3-phosphate dehydrogenase (G3PDH) loading control. The standardized fold change of nonsense transcripts was divided by the standardized fold change of the corresponding wild-type transcripts, and the resulting number was termed the NEI. The NEI values, ranging from 7.7 to 54.9, demonstrated that the nonsense transcripts were selectively stabilized in response to emetine treatment.

Use of GINI to Defect Nonsense Transcript in Colon Cancer Cell Line HCT116

The HCT116 cell line and three control fibroblast cell lines (CON1-3) were used to determine if a nonsense transcript could be identified using GINI. Each cell line was incubated for 10 h in fresh untreated medium or in medium with 100 ug/ml emetine, and mRNA was isolated. Unigem V microarrays containing 7,073 elements were used to analyze the changes induced by emetine treatment for each of the four cell lines. Expression changes were recorded as a fold change in which values >1.0 represent increases and values <1.0 represent decreases. As stated by the manufacturer, the threshold limit of detection in fold change is 1.7; anything less should be considered background (see http://www.incyte.com/reagents/gem/products-.shtml.). Therefore, all fold changes within a range of 0.588 (equivalent to a 1.7-fold decrease) to 1.7 were converted to 1.0 to reflect an undetectable change in transcript abundance. To identify genes that are normally upregulated by emetine treatment, an average fold change was calculated for each transcript for the three control lines, termed the average control score (ACS). The entire set of genes was then ranked according to the ACS in descending order (see Table 1 below). A total of 271 genes (3.83% of the total set) were found to have fold changes of 1.70 or higher in all three lines, implying that these genes have a predictable increase in expression due to treatment with emetine. The number of genes that ranked in the top 25 in each control cell line as well as in the ACS ranking was high (19, 20, and 21 out of 25). This demonstrates consistency among the transcripts that are highly upregulated by emetine treatment and indicates that the background emetine response can be efficiently subtracted to enrich for potential nonsense transcripts in the GINI strategy. To subtract the background, the fold change for each transcript from the tumor line HCT116 was divided by the ACS to calculate the NEI. The entire gene set was then ranked by this score in descending order (see Table 2 below). The MLH1 gene demonstrated no detectable change in expression by microarray analysis in the control lines and had a fold change of 3.35 in the HCT116 test line. Despite the fact that the actual change in MLH1 transcript levels in HCT116 was underestimated by the microarray (when compared to the Northern blots, see FIG. 2), a NEI of 3.35 was sufficient to give it a final ranking of 19th out of 7073 genes represented on the array. To illustrate the potential synergy of GINI with a positional cloning strategy in which the gene's chromosome identity has been predetermined, all genes known to reside on chromosome 3, where MLH1 had been previously mapped13, were selected and ranked based on their NEI score. Following this combination of strategies, the MLH1 gene ranked 3rd out of 197 chromosome 3 genes on the Unigem V microarray and in the top 0.04% overall.

Use of GINI to Detect a Known Nonsense Transcript in SANDHOFF CELL LINE 203

The GINI strategy was next used on the 203 cell line carrying the mutation HEXB/PTC, but in this case, the HUGENEFL array, containing 5,532 genes, were used to monitor the changes in mRNA expression. Because this chip has a twofold limit of detection (Research Genetics, Rhonda Snyder, personal communication), all fold changes below 2 and above 0.5 were recorded as 1.0 to reflect the absence of a detectable change in expression. Two control cell lines CON2 and CON4 were used to identify the background response to emetine treatment. Similar to the effects seen in HCT116, 316 transcripts, or 5.7% of the total, demonstrated a fold increase of >2.0 in the lines, again indicating that a small percentage of physiological transcripts are consistently upregulated by emetine treatment.

After dividing the transcript-specific fold change in the 203 line by the ACS of cell lines CON2 and CON4, a list of candidates was produced in which the HEXB gene was ranked 48th out of 5,532. A complete listing of the top 50 genes is available as Supplementary Table 1 in the Web Extras page of Nature Biotechnology Online. When combined with a positional strategy in which the chromosome of the Sandhoff locus, chromosome 5 (see Gilbert et al. (1975) PNAS USA 72: 263-7), is preselected, the HEXB gene ranked 3rd out of 187 known chromosome 5 genes on the HUGENEFL array and in the top 0.05% overall. When combined with functional information derived from consideration of the phenotype, it is clear that the hexosaminidase B gene would have been identified as the Sandhoff disease gene on the basis of GINI analysis.

Inhibition of Nonsense-Mediated mRNA Decay Using RNA-Interference—

RNA-interference (RNAi) refers to the potent inhibition of gene expression that occurs when double-stranded RNA of the same sequence as the gene is introduced into cells (Fire et al., (1998) Nature 391: 806-11; and Sharp (1999) Cell 76: 1091-98). RNAi is mediated by 21-nucleotide double-stranded RNA molecules, known as short-interfering RNAs (siRNAs), which induce degradation of cognate mRNAs via a poorly characterized mechanism (see Elbashir et al. (2001) Genes Dev 15: 188-200). It has recently been demonstrated that introducing synthetic siRNAs into mammalian tissue-culture cells using standard transfection techniques can induce potent knock-down of target messages (see Elbashir et al. (2001) Nature 411: 494-98). We applied this technology to the study of mammalian NMD to address whether the pathway could be inhibited by RNAi directed against RENT1 and rent2, two proteins believed to be essential for the process.

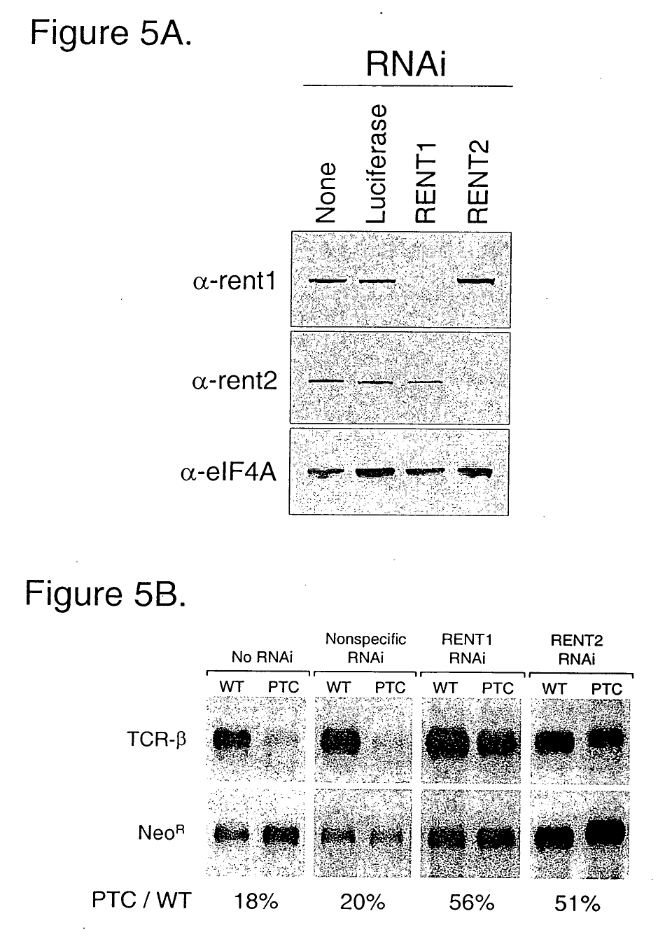

Short-interfering RNA (siRNA) targeting duplexes were designed to inhibit expression of RENT1 and rent2 and introduced into HeLa cells. Western blot analysis was used to monitor the resulting effect on protein expression (see FIG. 5A). Transfection with siRNA duplexes directed an unrelated protein (luciferase) had no effect on rent1 and rent2 expression. In contrast, RNAi directed against RENT1 resulted in a greater than 90% reduction in rent1 protein levels. Anti-rent1 siRNA duplexes had no effect on expression of rent2 or the translation initiation factor eIF4A, demonstrating their specificity. siRNA duplexes directed against rent2 showed a similar level of specific rent2 knockdown without detectable effects on rent1 or eIF4A protein expression. Thus, RNAi potently and specifically inhibits rent1 and rent2 expression in mammalian tissue culture.

The efficiency of NMD was assessed in cells lacking significant rent1 or rent2 expression by transfecting RNAi-treated cells with either a wild-type or a nonsense-containing mini-gene consisting of three exons of the T-cell receptor-b (TCR-b) gene (FIG. 5B). This transcript has previously been shown to be a substrate of the NMD pathway (see Li et al. (1997) J Exp Med 185: 985-992). Northern blot analysis was used to determine the steady-state level of the wild-type and mutant transcripts. The level of the NeoR transcript, encoded by the same plasmid carrying the TCR-b mini-gene, was used to control for differences in transfection efficiency and loading. In cells which did not receive siRNA targeting duplexes, the mutant transcript was reduced to 18% of wild-type levels, indicating baseline activity of the NMD pathway. An siRNA duplex directed against luciferase (nonspecific RNAi) did not significantly affect the level of the mutant transcript. In contrast, siRNA directed against rent1 or rent2 significantly stabilized the mutant transcript to greater than 50% of wild-type levels. This level of stabilization was consistent between multiple independent experiments. These results demonstrate that RNAi can effectively inhibit the NMD pathway in mammalian cells and suggests that RNAi may be an effective strategy to inhibit the pathway prior to GINI analysis.

Inhibition of the NMD Pathway Through Expression of a Dominant Negative Form of Rent1

Yet another strategy to inhibit the NMD pathway in mammalian cells involves the expression of mutant trans-effectors which can dominantly interfere with the normal function of the surveillance complex. After our laboratory identified the RENT1 gene (see Perlick et al., (1996) PNAS USA 93: 10928-32), we introduced a mutation that confers a dominant negative phenotype when introduced at the equivalent position in the yeast orthologue of rent1. Using this dominant negative form of rent1 to inhibit NMD, we demonstrated that this dominant-negative mutant form of rent1, which harbors an arginine-to-cysteine mutation at amino acid 844, acts in a dominant negative fashion and partially abrogates the accelerated decay of nonsense-containing beta-globin and glutathione peroxidase 1 (GP×1) transcripts (see Sun et al., (1998) PNAS USA 95: 1009-10014). Thus, overexpression of this dominant-negative form of rent1 may be an effective method to inhibit the NMD pathway prior to GINI analysis.

Tables

TABLE 1
Top 25 genes representing background
response to emetine treatment^{a}

| | | GenBank | | | | |
|---|---|---|---|---|---|---|
| Rank | Gene name | accession no. | CON1 | CON2 | CON3 | ACS |
| 1 | Early growth response protein 1 | X52541 | 67.1 | 39.5 | 38.9 | 48.49 |
| 2 | Hormone receptor (growth factor- | D49728 | 22.2 | 29.4 | 41.4 | 31.03 |
| | inducible nuclear protein N10) | | | | | |
| 3 | Putative DNA-binding protein A20 | M59465 | 38.6 | 16.0 | 38.4 | 31.01 |
| 4 | Early growth response protein 2 | J04076 | 34.4 | 26.5 | 29.5 | 30.15 |
| 6 | p55-c-fos proto-oncogene | V01512 | 35.5 | 17.3 | 23.4 | 25.42 |
| 7 | Major histocompatibility complex | M69043 | 28.1 | 21.7 | 22.3 | 24.05 |
| | enhancer-binding protein MAD3 | | | | | |
| 8 | Gem GTPase | U10550 | 24.4 | 18.8 | 12.1 | 18.43 |
| 9 | Transcription factor RELB | M83221 | 21.9 | 13.0 | 13.5 | 16.16 |
| 10 | Spermidine/spermine N1- | U40369 | 27.8 | 8.1 | 11.9 | 15.94 |
| | acetyltransferase | | | | | |
| 11 | Thyroid hormone receptor, a | M24898 | 16.9 | 13.3 | 13.1 | 14.43 |
| 12 | DNA-damage-inducible transcript 1 | L24498 | 17.8 | 12.4 | 12.4 | 14.19 |
| 13 | Dual-specificity protein phosphatase | L11329 | 11.8 | 9.8 | 21.0 | 14.17 |
| | PAC-1 | | | | | |
| 14 | Interferon regulatory factor 1 | X14454 | 16.9 | 6.7 | 14.0 | 12.52 |
| 15 | Interleukin 1, a | M28983 | 12.9 | 6.1 | 15.5 | 11.50 |
| 16 | V-abl Abelson murine leukemia viral | M35296 | 8.6 | 11.8 | 11.9 | 10.74 |
| | oncogene homolog 2 | | | | | |
| 17 | DEC1 | AB004066 | 10.8 | 12.6 | 7.8 | 10.38 |
| 18 | Diphtheria toxin receptor | M60278 | 12.4 | 7.9 | 10.0 | 10.09 |
| 19 | Early growth response protein 3 | X63741 | 9.0 | 10.3 | 9.9 | 9.75 |
| 20 | Putative transmembrane protein NMA | U23070 | 17.7 | 7.7 | 2.7 | 9.35 |
| 21 | Peptidyl-prolyl cis-trans isomerase | M80254 | 14.1 | 9.5 | 3.7 | 9.11 |
| 22 | IAP homolog C MIHC | U37546 | 9.6 | 8.5 | 8.0 | 8.72 |
| 23 | Thyroid receptor interactorTRIP9 | L40407 | 10.1 | 6.5 | 8.8 | 8.46 |
| 24 | Natural killer cells protein 4 precursor | M59807 | 14.2 | 5.7 | 4.7 | 8.23 |
| 25 | Small inducible cytokine A2 | M26683 | 9.7 | 3.6 | 10.5 | 7.92 |
| | Cutoff score to make top 25 list | | 10.1 | 7.0 | 8.9 | |
| | Number of genes in both individual | | 21/25 | 20/25 | 19/25 | |
| | top 25 and average top 25 | | | | | |

^{a}Abbreviation: ACS, average control score.

TABLE 2
Top 30 ranking genes from HCT-116 test line
following division by average control score

| | | GenBank | | | | | | | |
|---|---|---|---|---|---|---|---|---|---|
| | | accession | | | | | | | |
| Rank | Gene name | no. | Map | CON1 | CON2 | CON3 | ACS | HCT | NEI^{a} |
| 1 | Collagen, type I, \acute{α}1 | Z74615 | 17g21.3-q22 | 0.3 | 0.4 | 0.4 | 0.3 | 4.8 | 13.8 |
| 2 | Laminin, β2 | X79683 | 3p21 | 0.4 | 0.5 | 0.4 | 0.4 | 2.9 | 6.7 |
| 3 | Integrin, \acute{α}5 | X06256 | 12q11-q13 | 0.5 | 1.0 | 0.5 | 0.7 | 3.8 | 5.8 |
| 4 | Unknown | AB007890 | | 0.4 | 0.4 | 0.4 | 0.4 | 2.0 | 5.0 |
| 5 | FIP2 | AA595746 | 10 | 1.0 | 1.0 | 1.0 | 1.0 | 4.9 | 4.9 |
| 6 | Meisl-related | U68385 | | 1.0 | 2.5 | 1.0 | 1.5 | 7.0 | 4.6 |
| | protein | | | | | | | | |
| | 2 MRG2 | | | | | | | | |
| 7 | Stromal cell- | L36033 | 10q11.1 | 0.1 | 0.2 | 0.3 | 0.2 | 1.0 | 4.6 |
| | derived factor 1 | | | | | | | | |
| 8 | KIAA0151 | D63485 1 | 1 | 1.9 | 1.0 | 1.0 | 1.3 | 6.0 | 4.6 |
| 9 | MN1 | X82209 | 22q12.1 | 0.5 | 0.5 | 0.2 | 0.4 | 1.7 | 4.4 |
| 10 | hbc647 | U68494 | | 1.0 | 2.7 | 5.0 | 2.9 | 11.8 | 4.1 |
| 11 | Golgi antigen | X75304 | 3q13 | 1.0 | 1.0 | 1.0 | 1.0 | 4.0 | 4.0 |
| | gcp372 | | | | | | | | |
| 12 | Unknown | AA482549 | | 5.2 | 3.7 | 4.2 | 4.4 | 17.0 | 3.9 |
| 13 | Antigen peptide | X57522 | 6p21.3 | 1.0 | 1.0 | 1.0 | 1.0 | 3.8 | 3.8 |
| | transporter 1 | | | | | | | | |
| 14 | hkf-1 | D76444 | 2p11.2 | 1.0 | 1.0 | 1.0 | 1.0 | 3.8 | 3.8 |
| 15 | Hypothetical | AF000152 | 12g13-q15 | 0.5 | 1.0 | 1.0 | 0.8 | 3.1 | 3.7 |
| | protein A4 | | | | | | | | |
| 16 | Unknown | AA150408 | | 1.0 | 1.0 | 1.0 | 1.0 | 3.5 | 3.5 |
| 17 | Bruton's tyrosine | AF035737 | 7q11.23 | 0.2 | 0.4 | 0.3 | 0.3 | 1.0 | 3.5 |
| | kinase-associated | | | | | | | | |
| | protein-135 | | | | | | | | |
| 18 | HIV type I | X51435 - | 6p24-p22.3 | 1.0 | 1.9 | 3.3 | 2.0 | 6.9 | 3.4 |
| | enhancer-binding | | | | | | | | |
| | protein 1 | | | | | | | | |
| 19 | DNA mismatch | U07418 | 3p21.3 | 1.0 | 1.0 | 1.0 | 1.0 | 3.4 | 3.4 |
| | repair protein | | | | | | | | |
| | MLH1 | | | | | | | | |
| 20 | Immediate early | X75918 | 2q22-q23 | 3.4 | 6.3 | 2.4 | 4.0 | 13.4 | 3.3 |
| | response protein | | | | | | | | |
| | NOT | | | | | | | | |
| 21 | Unknown | W73588 | | 2.3 | 1.0 | 1.0 | 1.4 | 4.7 | 3.3 |
| 22 | cAMP- | S68271 | 10p12.1-p11.1 | 1.0 | 1.7 | 1.0 | 1.2 | 4.0 | 3.2 |
| | responsive | | | | | | | | |
| | element | | | | | | | | |
| | modulator | | | | | | | | |
| 23 | Tyrosine kinase | M76125 | 19q13.1 | 1.0 | 1.0 | 1.0 | 1.0 | 3.2 | 3.2 |
| | receptor | | | | | | | | |
| 24 | 218 kDa Mi-2 | X86691 | 12p13 | 1.0 | 1.0 | 1.0 | 1.0 | 3.2 | 3.2 |
| 25 | Scaffold | D50928 | | 1.0 | 2.1 | 1.0 | 1.4 | 4.3 | 3.2 |
| | attachment factor | | | | | | | | |
| | B SAF-B | | | | | | | | |
| 26 | Nuclear orphan | U22662 | | 1.0 | 1.0 | 1.0 | 1.0 | 3.2 | 3.2 |
| | receptor LXR-\acute{α} | | | | | | | | |
| 27 | Prostate | L78132 | | 1.0 | 1.0 | 0.6 | 0.9 | 2.7 | 3.2 |
| | carcinoma tumor | | | | | | | | |
| | antigen (pcta-1) | | | | | | | | |
| 28 | Insulin-like | L27560 | | 0.2 | 0.3 | 0.4 | 0.3 | 1.0 | 3.2 |
| | growth factor | | | | | | | | |
| | binding protein | | | | | | | | |
| | 5 IGFBP5 | | | | | | | | |
| 29 | Skeletal muscle | AF016270 | 5 | 1.0 | 1.0 | 1.0 | 1.0 | 3.1 | 3.1 |
| | abundant protein | | | | | | | | |
| 30 | AHNAK | M80899 | 11q12-q13 | 0.2 | 0.4 | 0.4 | 0.3 | 1.0 | 3.1 |
| | nucleoprotein | | | | | | | | |
| | (desmoyokin) | | | | | | | | |

^{a}Abbreviation: NEI, nonsense enrichment index. Values were calculated using raw data.

### DETAILED DESCRIPTION OF THE FIGURES

FIG. 1

FIG. 1 shows the effects of drugs on nonsense and wild-type transcripts. (A) Two cell lines, 203 and PC3, containing nonsense transcripts HEXB/PTC and TP53/PTC, respectively, were incubated for 10 h with the indicated doses of eight drugs. Transcript levels were standardized to the 7S ribosomal RNA (rRNA) loading control, then normalized to the level of the corresponding wild-type transcript from the untreated control cell line CON2. (B) Control cell line CON2 was treated with the indicated drug concentrations (ug/ml) for 10 h. The drugs represented are anisomycin (ANI), cycloheximide (CHX), emetine (EMT), pactamycin (PAC), and puromycin (PURO). Wild-type transcript levels for HEXB and TP53 were standardized to the 7S rRNA loading control, then normalized to the level of the corresponding wild-type transcript from untreated CON2 cells. (C) Time course of emetine treatment. 203 and PC3 cells were treated with 100 ug/ml emetine, and the steady-state levels of the HEXB/PTC and TP53/PTC transcripts were measured over time. Levels of transcripts were standardized to the 7S rRNA loading control. Ratios were then normalized to the levels of the untreated transcript (time point 0). Values on the y-axis correspond to the fold change in transcript levels over time. Each data point represents the average of three trials, and error bars show the standard deviation.

FIG. 2

FIG. 2 shows the stabilization of nonsense transcripts with emetine. Northern blots show the mRNA levels of four nonsense transcripts, and corresponding wild-type transcripts, in (U) untreated cells and (T) cells treated with 100 ug/ml emetine for 10 h. Numbers in the “fold” columns represent fold changes after standardizing to the G3PDH loading control. The NEI indicates the fold change of the nonsense transcript divided by the fold change of the wild-type transcript. Cell lines containing wild-type transcripts from top to bottom are CON2, CON3, LnCAP, and HCT116, whereas nonsense cell lines are 203, HCT116, PC3, and DLD1.

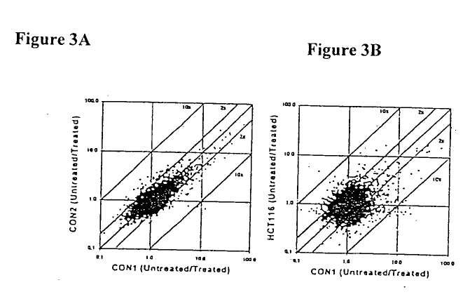

FIG. 3

FIG. 3 shows a comparison of transcript-specific responses to emetine in various cell lines. (A) Examination of two control primary fibroblast cell lines (CON1 and CON2). Each point represents a unique transcript that was represented on the microarray. The high density of points with an untreated:treated ratio of 1 manifests the lack of response of most mRNAs. A high degree of concordance between cell lines for a given transcript is also evident. (B) Comparison of the performance of transcripts in the CON1 and HCT116 cell lines.

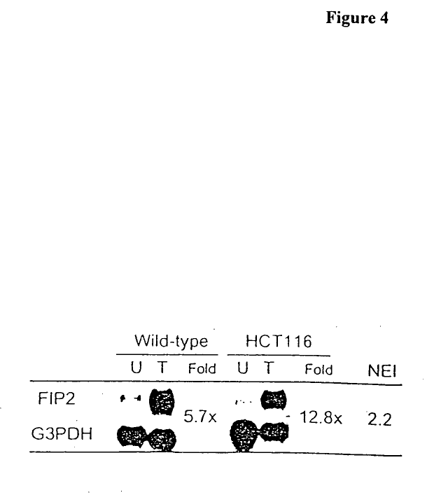

FIG. 4

FIG. 4 shows the response of FIP2 transcripts to emetine. Northern blots show the steady-state abundance of FIP2 mRNA in the CONS (wild type) and HCT116 cell lines in the untreated (U) state and after treatment (T) with 100 ug/ml emetine for 10 h. Numbers in the “fold” columns represent fold changes after standardizing to the G3PDH loading control. The NEI is calculated by dividing the fold change in the experimental cell line (HCT116) by the fold change in the control line (wild type).

FIG. 5

FIG. 5 shows that inhibition of NMD may be achieved using RNA interference (RNAi) to inhibit expression of NMD pathway genes RENT1 or RENT2. FIG. 5A shows that RNAi using siRNAs duplexes derived from RENT1 specifically depleted rent1 protein levels but not rent2 protein levels while siRNAs duplexes derived from RENT2 specifically depleted rent2 protein levels but not rent1 protein levels. Anti-RENT siRNAs did not interfere with an unrelated transcipt (eIF4A), nor did unrelated siRNAs (i.e. directed against the luciferase gene) interfere with rent1 or 2 expression. FIG. 5B shows that both anti-RENT1 and anti-RENT2 siRNAs were effective in inhibiting NMD-mediated inhibition of TCR-beta mRNA instability.

Discussion

We present GINI as a method of gene identification that exploits a fundamental and discriminating property of a broad class of mutant mRNAs. It provides a potentially powerful mechanism to associate a nucleotide sequence with a cellular or clinical phenotype of interest, even in the absence of any information regarding gene location or the function of the encoded peptide. As is apparent from the reported results, GINI provides an approach for rapidly identifying genes underlying previously uncharted human genetic diseases and disorders. It is also apparent that emerging technologies such as genome sequencing and annotation, expression profiling analysis, and mutation screening, will further facilitate still other GINI applications. GINI provides a quick and relatively inexpensive screen that has the potential for immediate success for disorders that might be otherwise unapproachable.

The basis for high NEI scores for multiple transcripts in our trials of GINI is likely heterogeneous. In an attempt to determine the extent to which this manifests biological noise (polymorphic or cell line-specific variation in the response to emetine) versus artifactual noise (reflecting current limitations in expression profiling technology), we compared the transcript-specific response to drug in multiple cell lines. Examination of primary fibroblasts revealed that the vast majority of transcripts do not change in abundance, and those that do generally show a concordant response between cell lines (FIG. 3A and data not shown). Reassuringly, the same pattern was observed when cells as diverse as primary fibroblasts and colon cancer cells were compared (FIG. 3B). Chip-based expression profiling methods often fail to detect low (or absent) levels of a given transcript and can assign an artificially high value for such mRNAs. Indeed, the absolute value for MLH1 in untreated colon cancer cells was actually slightly higher than that assigned to untreated fibroblasts despite clear northern blot data to the contrary (FIG. 2 and data not shown). Chip analysis can also fail to measure the accurate level of an abundant transcript, often attributed to a limiting amount of immobilized template for a given mRNA. Both factors may have contributed to inaccurately low estimates of the NEI for the disease genes of interest in both proof-of-concept experiments (3.4 for chip analysis versus 11.0 by quantitative northern analysis for MLH1; 12.1 versus 43.4 for HEXB). Substitution of the true (northern-derived) NEIs would have put these genes at the top of the list of candidates in our GINI analyses.

Adjunct information will be support the successful application of GINI, including the inferred or known biological function of candidate genes or, occasionally, a known map position for a given phenotype. In an objective assessment of our GINI results, the genes encoding a DNA mismatch repair factor (MLH1) or hexosaminidase B would have been clear favorites for patients with colon cancer or lysosomal accumulation of glycolipids, respectively, even in the absence of other a priori information. Leading candidates should be further scrutinized by quantitative reverse transcription (RT) PCR or northern analysis in both treated and untreated samples. For example, the FIP2 transcript, which ranked higher than MLH1 after chip-based analysis of HCT116 cells (Table 2), was not as promising when assessed by northern blot (FIG. 4). Findings included the absence of a striking deficiency in untreated HCT116 cells and a similar degree of upregulation in response to emetine in control cell lines, resulting in a corrected NEI value of 2.2, well below that determined for MLH1 by either microarray or northern anaylsis (3.4 and 11.0 respectively).

We considered several alternative methods to selectively enrich for nonsense transcripts, including recombinant expression of a dominant negative form of rent1, the mammalian ortholog of the essential yeast regulator of NMD, Upf1p (see Perlick et al. (1996) PNAS USA 93: 10928-32). This results in at least a modest (two- to threefold) upregulation of nonsense transcripts in mammalian cells (Sun et al. (1998) PNAS USA 95: 10009-10014), considerably below that achieved with emetine. The finding that rent1 appears essential for mammalian cellular viability (Medghalchi et al. (2001) Hum Mol Genet 10: 99-105) may support further refinement of the dominant negative approach. Second, targeted deletion of any of the Upf proteins in yeast results in disregulation of 8% of the yeast transcriptome due to both direct effects on the stability of selected physiological transcripts and indirect effects that boost transcription (Lelivelt et al. (1999) Mol Cell Biol 19: 671-19). Thus, perturbation of NMD through direct manipulation of its trans-effectors initially appeared to be more cumbersome than pharmacological methods and would not necessarily ensure a greater specificity for GINI. Nevertheless, we demonstrated that another dominant negative form of RENT1 carrying an arg to cys mutation at amino acid 844, acts to suppress NMD and may also be utilized for GINI analysis.

An important consideration when utilizing GINI is that not all nonsense transcripts are substrates for degradation by NMD. If a PTC is to induce nonsense decay, it must lie upstream of a point on the transcript that is 50 base pairs in the 5′ direction from the final exon/exon junction after splicing has occurred according to one study (see Nagy and Maquat (1998) Trends Biochem Sci 23: 198-99). For example, many nonsense codons in the adenomatous polyposis of the colon (APC) gene lie in the final exon and do not induce NMD (see Polakis (1995) Curr Opin Genet Dev 5: 66-71). Nevertheless, the majority of nonsense codons are predicted to initiate NMD and those which do not would not create a particular burden in light of the ability to rapidly identify those that do.

GINI requires that the relevant transcript is normally expressed in the tissue type from which the cell line is derived. This ensures that the nonsense transcript will have the opportunity to be increased in abundance through emetine treatment. Reassuringly, it has been shown that illegitimate transcripts are also substrates for NMD (Freddi et al. (2000) Am J Med Genet 90: 398-406; and Bateman et al. (1999) Hum Matat 13: 311-17), and, accordingly, this would allow detection of nonsense alleles even in cases where the transcript is not functionally important in the experimental cell line. The optimal target diseases for the GINI strategy include recessive disorders and cancers, which are most likely to be associated with homozygosity or hemizygosity for loss-of-function alleles. Furthermore, a tumor sample may have multiple mutations, possibly allowing for simultaneous identification of several genes involved in disease pathogenesis. Dominant diseases, however, may be precluded from this type of analysis in some instances where the presence of one normal allele dictates a maximum expression increase of twofold, beyond the reliable range of some microarray sensitivity. Forthcoming methods with improved sensitivity will further facilitate applications of GINI to dominant disorders and complex traits due to single loss-of-function alleles at multiple loci.

Appendix of Sequences
RENT1 (GenBank Accession No. NM_002911) (SEQ ID NO. 5)

| | |
|---|---|
| 1 | agcggctggc ggcttcgagg ggagctgagg cgcggagggg ctcggcggca gcggcggcgg |
| 61 | ctcggcactg ttacctctcg gtccggctgg cgccggggcg ggcggtttgg tcctttccgg |
| 121 | gcgcgcgggg gcgacagcgg cagcgacccg aggcctgcgg cctaggcctc agcgcggcgg |
| 181 | cgggctcgag tgcagcgcgg aaccggcccg agggccctac ccggaggcac catgagcgtg |
| 241 | gaggcgtacg ggcccagctc gcagactctc actttcctgg acacggagga ggccgagctg |
| 301 | cttggcgccg acacacaggg ctccgagttc gagttcaccg actttactct tcctagccag |
| 361 | acgcagacgc cccccggcgg ccccggcggc ccgggcggtg gcggcgcggg aggcccgggc |
| 421 | ggcgcgggcg cgggcgctgc ggcgggacag ctcgacgcgc aggttgggcc cgaaggcatc |
| 481 | ctgcagaacg gggctgtgga cgacagtgta gccaagacca gccagttgtt ggctgagttg |
| 541 | aacttcgagg aagatgaaga agacacctat tacacgaagg acctccccat acacgcctgc |
| 601 | agttactgtg gaatacacga tcctgcctgc gtggtttact gtaataccag caagaagtgg |
| 661 | ttctgcaacg gacgtggaaa tacttctggc agccacattg taaatcacct tgtgagggca |
| 721 | aaatgcaaag aggtgaccct gcacaaggac gggcccctgg gggagacagt cctggagtgc |
| 781 | tacaactgcg gctgtcgcaa cgtcttcctc ctcggcttca tcccggccaa agctgactca |
| 841 | gtggtggtgc tgctgtgcag gcagccctgt gccagccaga gcagcctcaa ggacatcaac |
| 901 | tgggacagct cgcagtggca gccgctgatc caggaccgct gcttcctgtc ctggctggtc |
| 961 | aagatcccct ccgagcagga gcagctgcgg gcacgccaga tcacggcaca gcagatcaac |
| 1021 | aagctggagg agctgtggaa ggaaaaccct tctgccacgc tggaggacct ggagaagccg |
| 1081 | ggggtggacg aggagccgca gcatgtcctc ctgcggtacg aggacgccta ccagtaccag |
| 1141 | aacatattcg ggcccctggt caagctggag gccgactacg acaagaagct gaaggagtcc |
| 1201 | cagactcaag ataacatcac tgtcaggtgg gacctgggcc ttaacaagaa gagaatcgcc |
| 1261 | tacttcactt tgcccaagac tgactctgac atgcggctca tgcaggggga tgagatatgc |
| 1321 | ctgcggtaca aaggggacct tgcgcccctg tggaaaggga tcggccacgt catcaaggtc |
| 1381 | cctgataatt atggcgatga gatcgccatt gagctgcgga gcagcgtggg tgcacctgtg |
| 1441 | gaggtgactc acaacttcca ggtggatttt gtgtggaagt cgacctcctt tgacaggatg |
| 1501 | cagagcgcat tgaaaacgtt tgccgtggat gagacctcgg tgtctggcta catctaccac |
| 1561 | aagctgttgg gccacgaggt ggaggacgta atcatcaagt gccagctgcc caagcgcttc |
| 1621 | acggcgcagg gcctccccga cctcaaccac tcccaggttt atgccgtgaa gactgtgctg |
| 1681 | caaagaccac tgagcctgat ccagggcccg ccaggcacgg ggaagacggt gacgtcggcc |
| 1741 | accatcgtct accacctggc ccggcaaggc aacgggccgg tgctggtgtg tgctccgagc |
| 1801 | aacatcgccg tggaccagct aacggagaag atccaccaga cggggctaaa ggtcgtgcgc |
| 1861 | ctctgcgcca agagccgtga ggccatcgac tccccggtgt cttttctggc cctgcacaac |
| 1921 | cagatcagga acatggacag catgcctgag ctgcagaagc tgcagcagct gaaagacgag |
| 1981 | actggggagc tgtcgtctgc cgacgagaag cggtaccggg ccttgaagcg caccgcagag |
| 2041 | agagagctgc tgatgaacgc agatgtcatc tgctgcacat gtgtgggcgc cggtgacccg |
| 2101 | aggctggcca agatgcagtt ccgctccatt ttaatcgacg aaagcaccca ggccaccgag |
| 2161 | ccggagtgca tggttcccgt ggtcctcggg gccaagcagc tgatccttgt aggcgaccac |
| 2221 | tgccagctgg gcccagtggt gatgtgcaag aaggcggcca aggccgggct gtcacagtcg |
| 2281 | ctcttcgagc gcctggtggt gctgggcatc cggcccatcc gcctgcaggt ccagtaccgg |
| 2341 | atgcaccctg cactcagcgc cttcccatcc aacatcttct acgagggctc cctccagaat |
| 2401 | ggtgtcactg cagcggatcg tgtgaagaag ggatttgact tccagtggcc ccaacccgat |
| 2461 | aaaccgatgt tcttctacgt gacccagggc caagaggaga ttgccagctc gggcacctcc |
| 2521 | tacctgaaca ggaccgaggc tgcgaacgtg gagaagatca ccacgaagtt gctgaaggca |
| 2581 | ggcgccaagc cggaccagat tggcatcatc acgccctacg agggccagcg ctcctacctg |
| 2641 | gtgcagtaca tgcagttcag cggctccctg cacaccaagc tctaccagga ggtggagatc |
| 2701 | gccagtgtgg acgcctttca gggacgcgag aaggacttca tcatcctgtc ctgtgtgcgg |
| 2761 | gccaacgagc accaaggcat tggcttttta aatgacccca ggcgtctgaa cgtggccctg |
| 2821 | accagagcaa ggtatggcgt catcattgtg ggcaacccga aggcactatc aaagcagccg |
| 2881 | ctctggaacc acctgctgaa ctactataag gagcagaagg tgctggtgga ggggccgctc |
| 2941 | aacaacctgc gtgagagcct catgcagttc agcaagccac ggaagctggt caacactatc |
| 3001 | aacccgggag cccgcttcat gaccacagcc atgtatgatg cccgggaggc catcatccca |
| 3061 | ggctccgtct atgatcggag cagccagggc cggccttcca gcatgtactt ccagacccat |
| 3121 | gaccagattg gcatgatcag tgccggccct agccacgtgg ctgccatgaa cattcccatc |
| 3181 | cccttcaacc tggtcatgcc acccatgcca ccgcctggct attttggaca agccaacggg |
| 3241 | cctgctgcag ggcgaggcac cccgaaaggc aagactggtc gtgggggacg ccagaagaac |
| 3301 | cgctttgggc ttcctggacc cagccagact aacctcccca acagccaagc cagccaggat |
| 3361 | gtggcgtcac agcccttctc tcagggcgcc ctgacgcagg gctacatctc catgagccag |
| 3421 | ccttcccaga tgagccagcc cggcctctcc cagccggagc tgtcccagga cagttacctt |
| 3481 | ggtgacgagt ttaaatcaca aatcgacgtg gcgctctcac aggactccac gtaccaggga |
| 3541 | gagcgggctt accagcatgg cggggtgacg gggctgtccc agtattaaaa ggtggcggcg |
| 3601 | gaagagctaa gcaacgtggc ttagtccatc agcatcttat tctgggtaat aaaaaataaa |
| 3661 | aataaacgga tacctgtttt ccactgctaa aactgaagca ccactgtgtg agcaacagga |
| 3721 | agggagagcg cacgagggag aggagccgag gccgagcgcc ccctgctggc ccgcggcggc |
| 3781 | gaggagcaga gggagcggag gaggggccgg cccgcgggag ccgcggccac caggaggccc |
| 3841 | cgctccgtcc catcggggct gcggccaggg cggagggagg aagaccctca tctcagagta |
| 3901 | gccctttcct ctgttctttt atttcttttt ctctttgatt gaaaggggac tacgtcttag |
| 3961 | caggaaaaaa aacttcgcat ttctgtgccc gagcaggctc cttgcaaaga cagcagcgtg |
| 4021 | cggggcagag ccccgggagg gcgcgtctgt ccacgcctac cggacgcgcc gaggtcgcgc |
| 4081 | tgcctgtgtt ctccgagggc cttcatttaa agaaaataag ggtgttttgg gtttttctct |
| 4141 | ttgttttttt caagattctt ttaaaggagt actgaagaat actttcctaa gtttgtctct |
| 4201 | aaaatcttag cggtggacct gggagatttg agaagcttcc agaaacagtt taaacaagcc |
| 4261 | agcgctactg gagaagagga gcaacacctg tgccgcggcc ggaggagttt tgttgttggt |
| 4321 | tttagcttcc agtggcttct ttctgcgggg catcaggctg ctggggtagc cgcccgccga |
| 4381 | gcctggaagc tgctcgttct ccgctggact cagaagccaa gctgcttccc gcctagactc |
| 4441 | ggcgcagggc cccgcaccgg tgaggaaggt gcttttggcc ccattgcgag gggccttggc |
| 4501 | caggactggc cctgtggcca ggaggcgaga aggtggctgt tcccggattg acggcttttt |
| 4561 | cccgggggcc tttggaagat ttggtggaag gacaagaggg cctgtccctg tccccgtccc |
| 4621 | caggaggtac cgacagtccc tgtgctggtt agacacggag cgctgcacac cgaaagccca |
| 4681 | aattgggagc tctgcctgcc ggcaactttg ctgatggggt gattgctgct tctggggggt |
| 4741 | aaggaaacaa gttacagaaa ttaccgcgtt ctgtgtgaag ggactgaggg tgtggtgtca |
| 4801 | ttggcagagg gtcattttag gagagctgcc ccagcccctc gaacgcctgg cttggggtgt |
| 4861 | cattctgcct ggcggccagg cctccagctt cccctgcccc gggcctgggg ctgtcactgg |
| 4921 | ccctgatccg aacacctcca gattccggct tctacatggg acagacgggg acgcacaggc |
| 4981 | caccttcctt ctggcaggga ctcttattta ttcccattgc tctagggctt tcggtttccc |
| 5041 | cttcttccgg taggccgcgt agaggcatgc accgggtagg tttccgcggt gaccccgcgg |
| 5101 | cggcctgagg gacgctccct gccccatccc ggctgttggg ctgggccgct ttgcctctgc |
| 5161 | ttcgccctgt gctgtgttct ccagctttgt agcagcagcc ttgacaaacc caggcgcact |
| 5221 | gtaccaaggc aatgtaactt ttgattttcg gtcaatttaa gttcttttgt caccaaatat |
| 5281 | taataaacag ttttgacttc |

Dominant-negative RENT 1
(GenBank Accession No. NP_002902
carrying an Arg to Cys alteration
at amino acid 843) (SEQ ID NO. 6)

| | |
|---|---|
| 1 | msveaygpss qtltfldtee aellgadtqg sefeftdftl psqtqtppgg |
| | pggpggggag |
| 61 | gpggagagaa agqldaqvgp egilqngavd dsvaktsqll aelnfeedee dtyytkdlpi |
| 121 | hacsycgihd pacvvycnts kkwfcngrgn tsgshivnhl vrakckevtl hkdgplgetv |
| 181 | lecyncgcrn vfllgfipak adsvvvllcr qpcasqsslk dinwdssqwq pliqdrcfls |
| 241 | wlvkipseqe qlrarqitaq qinkleelwk enpsatledl ekpgvdeepq hvllryeday |
| 301 | qyqnifgplv kleadydkkl kesqtqdnit vrwdlglnkk riayftlpkt dsdmrlmqgd |
| 361 | eiclrykgdl aplwkgighv ikvpdnygde iaielrssvg apvevthnfq vdfvwkstsf |
| 421 | drmqsalktf avdetsvsgy iyhkllghev edviikcqlp krftaqglpd lnhsqvyavk |
| 481 | tvlqrplsli qgppgtgktv tsativyhla rqgngpvlvc apsniavdql tekihqtglk |
| 541 | vvrlcaksre aidspvsfla lhnqirnmds mpelqklqql kdetgelssa dekryralkr |
| 601 | taerellmna dvicctcvga gdprlakmqf rsilidestq atepecmvpv vlgakqlilv |
| 661 | gdhcqlgpvv mckkaakagl sqslferlvv lgirpirlqv qyrmhpalsa fpsnifyegs |
| 721 | lqngvtaadr vkkgfdfqwp qpdkpmffyv tqgqeeiass gtsylnrtea anvekittkl |
| 781 | lkagakpdqi giitpyegqr sylvqymqfs gslhtklyqe veiasvdafq grekdfiils |
| 841 | cvcanehqgi gflndprrln valtrarygv iivgnpkals kqplwnhlln yykeqkvlve |
| 901 | gplnnlresl mqfskprklv ntinpgarfm ttamydarea iipgsvydrs sqgrpssmyf |
| 961 | qthdqigmis agpshvaamn ipipfnlvmp pmpppgyfgq angpaagrgt pkgktgrggr |
| 1021 | qknrfglpgp sqtnlpnsqa sqdvasqpfs qgaltqgyis msqpsqmsqp glsqpelsqd |
| 1081 | sylgdefksq idvalsqdst yqgerayqhg gvtglsqy |

RENT2- variant 1
(GenBank Accession Nos. NM_080599) (SEQ ID NO. 7)

| | |
|---|---|
| 1 | gcatgccgca gggaagacga tcaggactgt ttttaatcgg gcagtcgcgc ggatggcctt |
| 61 | ttccctctcg cctccttccg ccccgccccc actctcagcc cggccgcgct gattgtcctg |
| 121 | ggtcacataa tgccagctga gcgtaaaaag ccagcaagta tggaagaaaa agactcttta |
| 181 | ccaaacaaca aggaaaaaga ctgcagtgaa aggcggacag tgagcagcaa ggagaggcca |
| 241 | aaagacgata tcaagctcac tgccaagaag gaggtcagca aggcccctga agacaagaag |
| 301 | aagagactgg aagatgataa gagaaaaaag gaagacaagg aacgcaagaa aaaagacgaa |
| 361 | gaaaaggtga aggcagagga agaatcaaag aaaaaagaag aggaagaaaa aaagaaacat |
| 421 | caagaggaag agagaaagaa gcaagaagag caggccaaac gtcagcaaga agaagaagca |
| 481 | gctgctcaga tgaaagaaaa agaagaatcc attcagcttc atcaggaagc ttgggaacga |
| 541 | catcatttaa gaaaggaact tcgtagcaaa aaccaaaatg ctccggacag ccgaccagag |
| 601 | gaaaacttct tcagccgcct cgactcaagt ttgaagaaaa atactgcttt tgtcaagaaa |
| 661 | ctaaaaacta ttacagaaca acagagagac tccttgtccc atgattttaa tggcctaaat |
| 721 | ttaagcaaat acattgcaga agctgtagct tccatcgtgg aagcaaaact aaaaatctct |
| 781 | gatgtgaact gtgctgtgca cctctgctct ctctttcacc agcgttatgc tgactttgcc |
| 841 | ccatcacttc ttcaggtctg gaaaaaacat tttgaagcaa ggaaagagga gaaaacacct |
| 901 | aacatcacca agttaagaac tgatttgcgt tttattgcag aattgacaat agttgggatt |
| 961 | ttcactgaca aggaaggtct ttccttaatc tatgaacagc taaaaaatat tattaatgct |
| 1021 | gatcgggagt cccacactca tgtctctgta gtgattagtt tctgtcgaca ttgtggagat |
| 1081 | gatattgctg gacttgtacc aaggaaagta aagagtgctg cagagaagtt taatttgagt |
| 1141 | tttcctccta gtgagataat tagtccagag aaacaacagc ccttccagaa tcttttaaaa |
| 1201 | gagtacttta cgtctttgac caaacacctg aaaagggacc acagggagct ccagaatact |
| 1261 | gagagacaaa acaggcgcat tctacattct aaaggggagc tcagtgaaga tagacataaa |
| 1321 | cagtatgagg aatttgctat gtcttaccag aagctgctgg caaattctca atccttagca |
| 1381 | gaccttttgg atgaaaatat gccagatctt cctcaagaca aaccaacacc agaagaacat |
| 1441 | gggcctggaa ttgatatatt cacacctggt aaacctggag aatatgactt ggaaggtggt |
| 1501 | atatgggaag atgaagatgc tcggaatttt tatgagaacc tcattgattt gaaggctttt |
| 1561 | gtcccagcca tcttgtttaa agacaatgaa aaaagttgtc agaataaaga gtccaacaaa |
| 1621 | gatgatacca aagaggcaaa agaatctaag gagaataagg aggtatcaag tcccgatgat |
| 1681 | ttggaacttg agttggagaa tctagaaatt aatgatgaca ccttagaatt agagggtgga |
| 1741 | gatgaagctg aagatcttac aaagaaactt cttgatgaac aagaacaaga agatgaggaa |
| 1801 | gccagcactg gatctcatct caagctcata gtagatgctt tcctacagca gttacccaac |
| 1861 | tgtgtcaacc gagatctgat agacaaggca gcaatggatt tttgcatgaa catgaacaca |
| 1921 | aaagcaaaca ggaagaagtt ggtacgggca ctcttcatag ttcctagaca aaggttggat |
| 1981 | ttgctaccat tttatgcaag attggttgct acattgcatc cctgcatgtc tgatgtagca |
| 2041 | gaggatcttt gttccatgct gaggggggat ttcagatttc atgtacggaa aaaggaccag |
| 2101 | atcaatattg aaacaaagaa taaaactgtt cgttttatag gagaactaac taagtttaag |
| 2161 | atgttcacca aaaatgacac actgcattgt ttaaagatgc ttctgtcaga cttctctcat |
| 2221 | caccatattg aaatggcatg caccctgctg gagacatgtg gacggtttct tttcagatct |
| 2281 | ccagaatctc acctgaggac cagtgtactt ttggagcaaa tgatgagaaa gaagcaagca |
| 2341 | atgcatcttg atgcgagata cgtcacaatg gtagagaatg catattacta ctgcaaccca |
| 2401 | cctccagctg aaaaaaccgt gaaaaagaaa cgtcctcctc tccaggaata tgtccggaaa |
| 2461 | cttttgtaca aggatctctc taaggttacc accgagaagg ttttgagaca gatgcgaaag |
| 2521 | ctgccctggc aggaccaaga agtgaaagac tatgttattt gttgtatgat aaacatctgg |
| 2581 | aatgtgaaat ataatagtat tcattgtgta gccaacctct tagcaggact agtgctctac |
| 2641 | caagaggatg ttgggatcca cgttgtggat ggagtgttag aagatattcg attaggaatg |
| 2701 | gaggttaatc aacctaaatt taatcagagg cgcatcagca gtgccaagtt cttaggagaa |
| 2761 | ctttacaatt accgaatggt ggaatcagct gttattttca gaactctgta ttcttttacc |
| 2821 | tcatttggtg ttaatcctga tggctctcca agttccctgg acccacctga gcatcttttc |
| 2881 | agaattagac tcgtatgcac tattctggac acatgtggcc agtactttga cagaggttcc |
| 2941 | agtaaacgaa aacttgattg tttccttgta tattttcagc gttatgtttg gtggaagaaa |
| 3001 | agtttggagg tttggacaaa agaccatcca tttcctattg atatagatta catgatcagt |
| 3061 | gatacactag aactgctaag accaaagatc aaactctgta attctctgga agaatccatc |
| 3121 | aggcaggtac aagacttgga acgagaattc ttaataaaac taggcctagt aaatgacaaa |
| 3181 | gactcaaaag attctatgac agaaggagaa aatcttgaag aggatgaaga agaagaagaa |
| 3241 | ggtggggctg aaacagaaga acaatctgga aatgaaagtg aagtaaatga gccagaagaa |
| 3301 | gaggagggtt ctgataatga tgatgatgag ggagaagaag aggaggaaga gaatacagat |
| 3361 | taccttacag attccaataa ggaaaatgaa accgatgaag agaatactga ggtaatgatt |
| 3421 | aaaggcggtg gacttaagca tgtaccttgt gtagaagatg aggacttcat tcaagctctg |
| 3481 | gataaaatga tgctagaaaa tctacagcaa cgaagtggtg aatctgttaa agtgcaccaa |
| 3541 | ctagatgttg ccattccttt gcatctcaaa agccagctga ggaaagggcc cccactggga |
| 3601 | ggtggggaag gagaggctga gtctgcagac acaatgccgt ttgtcatgtt aacaagaaaa |
| 3661 | ggcaataaac agcagtttaa gatccttaat gtacccatgt cctctcaact tgctgcaaat |
| 3721 | cactggaacc agcaacaggc agaacaagaa gagaggatga gaatgaaaaa gctcacacta |
| 3781 | gatatcaatg aacggcaaga acaagaagat tatcaagaaa tgttgcagtc tcttgcacag |
| 3841 | cgcccagctc cagcaaacac caatcgtgag aggcggcctc gctaccaaca tccgaaggga |
| 3901 | gcacctaatg cagatctaat ctttaagact ggtgggagga gacgttgatc cagcagcacg |
| 3961 | tgtcatttca ttaggtcctg tatctgatgt tgtggttagt ggagtcctcc agcaattgaa |
| 4021 | tgagagcagt ggacacatct cagcaggtcg gtctagagag ttgcgaatct aaacctggga |
| 4081 | caggctgggg ccaggaggca gaaacaccag cctctgccaa caccggaaca agccgacgct |
| 4141 | tccagacaag gcggaaaagg ccttttgtaa tggaaatctc gcgagggtta atcttctctt |
| 4201 | gagaatggca gtcaagaaat gagatggttc acttgactac tgagcagtta caccaaggag |
| 4261 | agcgtgaagg agatgattga gccagagaag aaacgggttg tgatggtaat ggtgtggggg |
| 4321 | aaatgaactt gagctttaaa cttgatttga gtttcagtgt ctctgaattg aacatcccac |
| 4381 | gttggaagaa gatacatttg ggggctccag gactacagta gaaaagtata gagcaagcag |
| 4441 | gaaaatcttc tagtaaaact tacatgcagg acaacaaaat gatgaaagat atccaaatac |
| 4501 | cagataatcc accaggaagg cttttgttta ggaatttgtt tcaagaggaa caagggatga |
| 4561 | gggagaaaaa tccgttttat ccatcagagt cagtgctata aaattgccta ttaaggtaaa |
| 4621 | agaaaaatgt ggagactatt ttactataca gagagcatta attcagatgg cttagaaaag |
| 4681 | tgataccagc ccaagaacag ggatctaggt gagcccattg taagtatcat tgaaaacaaa |
| 4741 | acatgcccgt caacatgtca cagaaaacga acgaaggaca acaagaagtg gatgagaata |
| 4801 | ttttgttgac cttcatgggt ttacagcctc tgtctctaaa caaagtatgg aaacaagtag |
| 4861 | agcttttatt ttgcttttgt ttttgttttg tttttttttt tgttttcccc cactaaatag |
| 4921 | aaatgagggt ccttagtctg tttctgacaa tctgttaatt tcttaggaca gctgtctttg |
| 4981 | gtttgctttc cagcaggcgt agtatattta gtcggagagc acatctgtat gcgacaactt |
| 5041 | gattacatct ttttttctag ctattttgca ttttttcttt taccatgttt cagtttctgc |
| 5101 | atgtagattt aaataaaaaa caaaacttgt aaagttgtaa catttcacat ggaaatgctg |
| 5161 | cccaatcttc accagcttca gaaatctgac ctttgccgat gctgcaataa agtgttgtaa |
| 5221 | ttt |

RENT2- variant 2
(GenBank Accession Nos. NM_015542) (SEQ ID NO. 8)

| | |
|---|---|
| 1 | gagcgctgga gttggtgctg ggaaacccgg ggctaatgtt gacaacaggc tcgagattgt |
| 61 | cctgggtcac ataatgccag ctgagcgtaa aaagccagca agtatggaag aaaaagactc |
| 121 | tttaccaaac aacaaggaaa aagactgcag tgaaaggcgg acagtgagca gcaaggagag |
| 181 | gccaaaagac gatatcaagc tcactgccaa gaaggaggtc agcaaggccc ctgaagacaa |
| 241 | gaagaagaga ctggaagatg ataagagaaa aaaggaagac aaggaacgca agaaaaaaga |
| 301 | cgaagaaaag gtgaaggcag aggaagaatc aaagaaaaaa gaagaggaag aaaaaaagaa |
| 361 | acatcaagag gaagagagaa agaagcaaga agagcaggcc aaacgtcagc aagaagaaga |
| 421 | agcagctgct cagatgaaag aaaaagaaga atccattcag cttcatcagg aagcttggga |
| 481 | acgacatcat ttaagaaagg aacttcgtag caaaaaccaa aatgctccgg acagccgacc |
| 541 | agaggaaaac ttcttcagcc gcctcgactc aagtttgaag aaaaatactg cttttgtcaa |
| 601 | gaaactaaaa actattacag aacaacagag agactccttg tcccatgatt ttaatggcct |
| 661 | aaatttaagc aaatacattg cagaagctgt agcttccatc gtggaagcaa aactaaaaat |
| 721 | ctctgatgtg aactgtgctg tgcacctctg ctctctcttt caccagcgtt atgctgactt |
| 781 | tgccccatca cttcttcagg tctggaaaaa acattttgaa gcaaggaaag aggagaaaac |
| 841 | acctaacatc accaagttaa gaactgattt gcgttttatt gcagaattga caatagttgg |
| 901 | gattttcact gacaaggaag gtctttcctt aatctatgaa cagctaaaaa atattattaa |
| 961 | tgctgatcgg gagtcccaca ctcatgtctc tgtagtgatt agtttctgtc gacattgtgg |
| 1021 | agatgatatt gctggacttg taccaaggaa agtaaagagt gctgcagaga agtttaattt |
| 1081 | gagttttcct cctagtgaga taattagtcc agagaaacaa cagcccttcc agaatctttt |
| 1141 | aaaagagtac tttacgtctt tgaccaaaca cctgaaaagg gaccacaggg agctccagaa |
| 1201 | tactgagaga caaaacaggc gcattctaca ttctaaaggg gagctcagtg aagatagaca |
| 1261 | taaacagtat gaggaatttg ctatgtctta ccagaagctg ctggcaaatt ctcaatcctt |
| 1321 | agcagacctt ttggatgaaa atatgccaga tcttcctcaa gacaaaccaa caccagaaga |
| 1381 | acatgggcct ggaattgata tattcacacc tggtaaacct ggagaatatg acttggaagg |
| 1441 | tggtatatgg gaagatgaag atgctcggaa tttttatgag aacctcattg atttgaaggc |
| 1501 | ttttgtccca gccatcttgt ttaaagacaa tgaaaaaagt tgtcagaata aagagtccaa |
| 1561 | caaagatgat accaaagagg caaaagaatc taaggagaat aaggaggtat caagtcccga |
| 1621 | tgatttggaa cttgagttgg agaatctaga aattaatgat gacaccttag aattagaggg |
| 1681 | tggagatgaa gctgaagatc ttacaaagaa acttcttgat gaacaagaac aagaagatga |
| 1741 | ggaagccagc actggatctc atctcaagct catagtagat gctttcctac agcagttacc |
| 1801 | caactgtgtc aaccgagatc tgatagacaa ggcagcaatg gatttttgca tgaacatgaa |
| 1861 | cacaaaagca aacaggaaga agttggtacg ggcactcttc atagttccta gacaaaggtt |
| 1921 | ggatttgcta ccattttatg caagattggt tgctacattg catccctgca tgtctgatgt |
| 1981 | agcagaggat ctttgttcca tgctgagggg ggatttcaga tttcatgtac ggaaaaagga |
| 2041 | ccagatcaat attgaaacaa agaataaaac tgttcgtttt ataggagaac taactaagtt |
| 2101 | taagatgttc accaaaaatg acacactgca ttgtttaaag atgcttctgt cagacttctc |
| 2161 | tcatcaccat attgaaatgg catgcaccct gctggagaca tgtggacggt ttcttttcag |
| 2221 | atctccagaa tctcacctga ggaccagtgt acttttggag caaatgatga gaaagaagca |
| 2281 | agcaatgcat cttgatgcga gatacgtcac aatggtagag aatgcatatt actactgcaa |
| 2341 | cccacctcca gctgaaaaaa ccgtgaaaaa gaaacgtcct cctctccagg aatatgtccg |
| 2401 | gaaacttttg tacaaggatc tctctaaggt taccaccgag aaggttttga gacagatgcg |
| 2461 | aaagctgccc tggcaggacc aagaagtgaa agactatgtt atttgttgta tgataaacat |
| 2521 | ctggaatgtg aaatataata gtattcattg tgtagccaac ctcttagcag gactagtgct |
| 2581 | ctaccaagag gatgttggga tccacgttgt ggatggagtg ttagaagata ttcgattagg |
| 2641 | aatggaggtt aatcaaccta aatttaatca gaggcgcatc agcagtgcca agttcttagg |
| 2701 | agaactttac aattaccgaa tggtggaatc agctgttatt ttcagaactc tgtattcttt |
| 2761 | tacctcattt ggtgttaatc ctgatggctc tccaagttcc ctggacccac ctgagcatct |
| 2821 | tttcagaatt agactcgtat gcactattct ggacacatgt ggccagtact ttgacagagg |
| 2881 | ttccagtaaa cgaaaacttg attgtttcct tgtatatttt cagcgttatg tttggtggaa |
| 2941 | gaaaagtttg gaggtttgga caaaagacca tccatttcct attgatatag attacatgat |
| 3001 | cagtgataca ctagaactgc taagaccaaa gatcaaactc tgtaattctc tggaagaatc |
| 3061 | catcaggcag gtacaagact tggaacgaga attcttaata aaactaggcc tagtaaatga |
| 3121 | caaagactca aaagattcta tgacagaagg agaaaatctt gaagaggatg aagaagaaga |
| 3181 | agaaggtggg gctgaaacag aagaacaatc tggaaatgaa agtgaagtaa atgagccaga |
| 3241 | agaagaggag ggttctgata atgatgatga tgagggagaa gaagaggagg aagagaatac |
| 3301 | agattacctt acagattcca ataaggaaaa tgaaaccgat gaagagaata ctgaggtaat |
| 3361 | gattaaaggc ggtggactta agcatgtacc ttgtgtagaa gatgaggact tcattcaagc |
| 3421 | tctggataaa atgatgctag aaaatctaca gcaacgaagt ggtgaatctg ttaaagtgca |
| 3481 | ccaactagat gttgccattc ctttgcatct caaaagccag ctgaggaaag ggcccccact |
| 3541 | gggaggtggg gaaggagagg ctgagtctgc agacacaatg ccgtttgtca tgttaacaag |
| 3601 | aaaaggcaat aaacagcagt ttaagatcct taatgtaccc atgtcctctc aacttgctgc |
| 3661 | aaatcactgg aaccagcaac aggcagaaca agaagagagg atgagaatga aaaagctcac |
| 3721 | actagatatc aatgaacggc aagaacaaga agattatcaa gaaatgttgc agtctcttgc |
| 3781 | acagcgccca gctccagcaa acaccaatcg tgagaggcgg cctcgctacc aacatccgaa |
| 3841 | gggagcacct aatgcagatc taatctttaa gactggtggg aggagacgtt gatccagcag |
| 3901 | cacgtgtcat ttcattaggt cctgtatctg atgttgtggt tagtggagtc ctccagcaat |
| 3961 | tgaatgagag cagtggacac atctcagcag gtcggtctag agagttgcga atctaaacct |
| 4021 | gggacaggct ggggccagga ggcagaaaca ccagcctctg ccaacaccgg aacaagccga |
| 4081 | cgcttccaga caaggcggaa aaggcctttt gtaatggaaa tctcgcgagg gttaatcttc |
| 4141 | tcttgagaat ggcagtcaag aaatgagatg gttcacttga ctactgagca gttacaccaa |
| 4201 | ggagagcgtg aaggagatga ttgagccaga gaagaaacgg gttgtgatgg taatggtgtg |
| 4261 | ggggaaatga acttgagctt taaacttgat ttgagtttca gtgtctctga attgaacatc |
| 4321 | ccacgttgga agaagataca tttgggggct ccaggactac agtagaaaag tatagagcaa |
| 4381 | gcaggaaaat cttctagtaa aacttacatg caggacaaca aaatgatgaa agatatccaa |
| 4441 | ataccagata atccaccagg aaggcttttg tttaggaatt tgtttcaaga ggaacaaggg |
| 4501 | atgagggaga aaaatccgtt ttatccatca gagtcagtgc tataaaattg cctattaagg |
| 4561 | taaaagaaaa atgtggagac tattttacta tacagagagc attaattcag atggcttaga |
| 4621 | aaagtgatac cagcccaaga acagggatct aggtgagccc attgtaagta tcattgaaaa |
| 4681 | caaaacatgc ccgtcaacat gtcacagaaa acgaacgaag gacaacaaga agtggatgag |
| 4741 | aatattttgt tgaccttcat gggtttacag cctctgtctc taaacaaagt atggaaacaa |
| 4801 | gtagagcttt tattttgctt ttgtttttgt tttgtttttt tttttgtttt cccccactaa |
| 4861 | atagaaatga gggtccttag tctgtttctg acaatctgtt aatttcttag gacagctgtc |
| 4921 | tttggtttgc tttccagcag gcgtagtata tttagtcgga gagcacatct gtatgcgaca |
| 4981 | acttgattac atcttttttt ctagctattt tgcatttttt cttttaccat gtttcagttt |
| 5041 | ctgcatgtag atttaaataa aaaacaaaac ttgtaaagtt gtaacatttc acatggaaat |
| 5101 | gctgcccaat cttcaccagc ttcagaaatc tgacctttgc cgatgctgca ataaagtgtt |
| 5161 | gtaatttaaa aaaaaaaaaa aaaaa |

### EQUIVALENTS

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

We claim:

1) a method of identifying a gene carrying a mutation that causes nonsense-mediated premature protein termination in a cell or cell population comprising:
providing a cell or cell population;
detecting the level of expression of a gene in said cell or cell population;
inhibiting nonsense-mediated mRNA decay in said cell or cell population; and
detecting an increase in the level of expression of the gene in said cell or cell population following inhibition of nonsense-mediated mRNA decay,
wherein an increase in the level of expression of the gene following inhibition of nonsense-mediated mRNA decay indicates that the gene carries a mutation that causes nonsense-mediated premature protein termination in the cell or cell population.

2) The method of claim 1, wherein nonsense-mediated mRNA decay is inhibited in the cell or cell population by contacting the cell or cell population with a pharmacological agent that interferes with the nonsense-mediated decay pathway.

3) The method of claim 2, wherein the pharmacological agent is an inhibitor of protein translation.

4) The method of claim 2, wherein the pharmacological agent is selected from the group consisting of: emetine, anisomycin, cycloheximide, pactamycin, puromycin, gentamicin, neomycin, and paromomycin.

5) The method of claim 1, wherein nonsense-mediated mRNA decay is inhibited in the cell or cell population by introduction of an siRNA comprising a sequence of consecutive nucleotides present in a component of the NMD pathway.

6) The method of claim 5, wherein the siRNA comprises a sequence of consecutive nucleotides present in a gene selected from the group consisting of RENT1 and RENT2.

7) The method of claim 6, wherein the siRNA comprises SEQ ID Nos. 1 and 2.

8) The method of claim 6, wherein the siRNA comprises SEQ ID Nos. 3 and 4.

9) The method of claim 1, wherein nonsense-mediated mRNA decay is inhibited in the cell or cell population by introduction of a dominant negative RENT1 or RENT2.

10) The method of claim 9, wherein the dominant negative RENT1 comprises an arg to cys mutation at the RENT1 amino acid residue 843.

11) The method of claim 10, wherein the dominant negative RENT1 comprises the polypeptide sequence of SEQ ID No. 6.

12) The method of claim 1, wherein nonsense-mediated mRNA decay is inhibited in the cell or cell population by introduction of an antisense nucleic acid directed against a RENT1 mRNA or a RENT2 mRNA.

13) The method of claim 1, wherein nonsense-mediated mRNA decay is inhibited in the cell or cell population by introduction of a ribozyme directed against a RENT1 mRNA or a RENT2 mRNA.

14) The method of claim 1, wherein the gene is an oncogene.

15) The method of claim 1, wherein the level of expression of the gene is detected by a method selected from the group consisting of: microarray analysis, quantitative pcr, SAGE analysis, Northern blot analysis and dot blot analysis.

16) A computer-readable medium comprising a plurality of digitally encoded information representing the genes having the strongest background response to inhibition of nonsense-mediated mRNA decay including a plurality of members of the group consisting of: early growth response protein 1, hormone receptor (growth factor-inducible nuclear protein N10), putative DNA-binding protein A20, early growth response protein 2, p55-c-fos proto-oncogene, major histocompatibility complex enhancer-binding protein MAD3, gem GTPase, transcription factor RELB, spermidine/spermine N1-acetyltransferase, thyroid hormone receptor, alpha; DNA-damage-inducible transcript 1, dual-specificity protein phosphatase PAC-1, interferon regulatory factor 1, interleukin 1, alpha, V-abl Abelson murine leukemia viral oncogene homolog 2, DEC1, diphtheria toxin receptor, early growth response protein 3, putative transmembrane protein NMA, peptidyl-prolyl cis-trans isomerase, IAP homolog C MIHC, thyroid receptor interactor TRIP9, natural killer cells protein 4 precursor and small inducible cytokine A2.

17) A computer-readable medium comprising a plurality of digitally encoded information representing the genes having the strongest background response to inhibition of nonsense-mediated mRNA decay including a plurality of members of the group consisting of GenBank Accession Nos.: X52541, D49728, M59465, J04076, M69043, U10550, M83221, U40369, M24898, L24498, L11329, X14454, M28983, M35296, AB004066, M60278, X63741, U23070, M80254, U37546, L40407, M59807 and M26683.

18) A method of identifying a candidate mutant gene in a cell or cell population that carries a genetic mutation that causes nonsense-mediated mRNA decay comprising:
providing a cell or cell population that carries a genetic mutation and measuring the level of expression of a plurality of genes in said cell or cell population, wherein the level of expression measured is the control level of expression of each gene;
determining the level of expression of the plurality of genes in said cell or cell population under conditions in which nonsense-mediated mRNA decay is inhibited; and
selecting a gene from the plurality of genes in which the control level of expression of the gene is lower than the level of expression under conditions that inhibit nonsense-mediated mRNA decay,
wherein the selected gene is a candidate mutant gene for the genetic mutation that causes nonsense-mediated mRNA decay is the cell or cell population.

19) The method of claim 18, wherein the genetic mutation causes or contributes to a human genetic disease or disorder.

20) The method of claim 18, wherein the gene selected is other than a gene selected from the group consisting of: early growth response protein 1, hormone receptor (growth factor-inducible nuclear protein N10), putative DNA-binding protein A20, early growth response protein 2, p55-c-fos proto-oncogene, major histocompatibility complex enhancer-binding protein MAD3, gem GTPase, transcription factor RELB, spermidine/spermine N1-acetyltransferase, thyroid hormone receptor, alpha; DNA-damage-inducible transcript 1, dual-specificity protein phosphatase PAC-1, interferon regulatory factor 1, interleukin 1, alpha, V-abl Abelson murine leukemia viral oncogene homolog 2, DEC1, diphtheria toxin receptor, early growth response protein 3, putative transmembrane protein NMA, peptidyl-prolyl cis-trans isomerase, IAP homolog C MIHC, thyroid receptor interactor TRIP9, natural killer cells protein 4 precursor and small inducible cytokine A2.

21) The method of claim 18, wherein the gene selected is other than a gene selected from the group consisting of GenBank Accession Nos.: X52541, D49728, M59465, J04076, M69043, U10550, M83221, U40369, M24898, L24498, L11329, X14454, M28983, M35296, AB004066, M60278, X63741, U23070, M80254, U37546, L40407, M59807 and M26683.

22) The method of claim 18, wherein nonsense-mediated mRNA decay is inhibited in the cell or cell population by contacting the cell or cell population with a pharmacological agent that interferes with the nonsense-mediated decay pathway.

23) The method of claim 22, wherein the pharmacological agent is an inhibitor of protein translation.

24) The method of claim 23, wherein the pharmacological agent is selected from the group consisting of: emetine, anisomycin, cycloheximide, pactamycin, puromycin, gentamicin, neomycin, and paromomycin.

25) The method of claim 18, wherein nonsense-mediated mRNA decay is inhibited in the cell or cell population by introduction of an siRNA comprising a sequence of consecutive nucleotides present in a component of the NMD pathway.

26) The method of claim 25, wherein the siRNA comprises a sequence of consecutive nucleotides present in a gene selected from the group consisting of RENT1 and RENT2.

27) The method of claim 26, wherein the siRNA comprises SEQ ID Nos. 1 and 2.

28) The method of claim 26, wherein the siRNA comprises SEQ ID Nos. 3 and 4.

29) The method of claim 18, wherein nonsense-mediated mRNA decay is inhibited in the cell or cell population by introduction of a dominant negative RENT1 or RENT2.

30) The method of claim 29, wherein the dominant negative RENT1 comprises an arg to cys mutation at the RENT1 amino acid residue 843.

31) The method of claim 30, wherein the dominant negative RENT1 comprises the polypeptide sequence of SEQ ID No. 6.

32) The method of claim 18, wherein nonsense-mediated mRNA decay is inhibited in the cell or cell population by introduction of an antisense nucleic acid directed against a RENT1 mRNA or a RENT2 mRNA.

33) The method of claim 18, wherein nonsense-mediated mRNA decay is inhibited in the cell or cell population by introduction of a ribozyme directed against a RENT1 mRNA or a RENT2 mRNA.

34) The method of claim 18, wherein the gene selected is an oncogene.

35) The method of claim 18, wherein the level of expression of the gene is detected by a method selected from the group consisting of: microarray analysis, quantitative pcr, SAGE analysis, Northern blot analysis and dot blot analysis.

36) A method of subtractive hybridization for identifying a candidate mutant gene in a cell line or cell population that carries a genetic mutation that causes nonsense-mediated mRNA decay comprising:
providing a cell population or a cell line that carries a genetic mutation,
forming a first cDNA population from mRNA that has been expressed by the cells under conditions in which nonsense-mediated mRNA decay is inhibited and a second cDNA population from mRNA that has been expressed by the cells under control conditions in which nonsense-mediated mRNA decay is not inhibited,
removing from the first cDNA population at least a portion of the cDNA common to the first and second populations by subtractive hybridization to provide enriched cDNA coding for genes that are differentially stabilized by inhibition of nonsense-mediated mRNA decay, and
identifying a gene in the resulting enriched cDNA population,
thereby identifying a candidate mutant gene in a cell line or cell population that carries a genetic mutation.

37) A library comprising a plurality of cDNA sequences coding for genes that are differentially stabilized by inhibition of nonsense-mediated mRNA decay.

38) The library of claim 37, wherein the plurality of cDNA sequences coding for genes that are differentially stabilized by inhibition of nonsense-mediated mRNA decay is obtained by the method of claim 36.

39) A method of determining whether a cellular phenotype that is associated with a disease or disorder results from a nonsense mutation comprising:
providing a cell or cell population having a cellular phenotype that is associated with a disease or disorder;
inhibiting nonsense mediated decay in said cell or cell population; and
detecting an alteration in said cellular phenotype following the inhibition of nonsense mediated decay in said cell or cell population
wherein an alteration (exacerbation—as in the case of C. elegans unc-54 strains in an smg minus background) in said cellular phenotype following the inhibition of nonsense mediated decay indicates that the cellular phenotype results from a nonsense mutation.
